(19) United States
(12) Patent Application Publication
(10) Pub. No.: US 2007/0270403 A1
(43) Pub. Date: Nov. 22, 2007

(54) Clozapine and Cocaine Effects on Dopamine and Serotonin Release in Nucleus Accumbens During Psychostimulant Behavior and Withdrawal

(76) Inventor: Patricia A. Broderick, Bronx, NY (US)

Correspondence Address: MORGAN & FINNEGAN, L.L.P., 3 WORLD FINANCIAL CENTER, NEW YORK, NY, 10281-2101, US

(21) Appl. No.: 10/580,884

(22) PCT Filed: Dec. 6, 2004
(86) PCT No.: PCT/US04/40756

§ 371 Date: May 4, 2007

Related U.S. Application Data
(60) Provisional application No. 60/526,833, filed on Dec. 4, 2003.

(51) Int. Cl. A61K 31/551; A61K 31/40; A61K 31/438; A61K 31/445; A61K 31/454; A61K 31/496; A61K 31/554; A61P 25/18; A61K 31/553; A61K 31/497; A61K 31/517; A61K 31/519; A61K 31/5415

(52) U.S. Cl. 514/211.13; 514/220; 514/225.5; 514/252.13; 514/253.07; 514/254.04; 514/259.41; 514/266.22; 514/278; 514/315; 514/323; 514/428

(57) ABSTRACT

The present invention provides methods of treating cocaine-induced pyschosis by administering an atypical antipsychotic compound in an amount sufficient to increase serotonin concentration in the nucleus accumbens of a mammal. According to the invention, atypical antipsychotic compounds include, without limitation, clozapine, risperidone, olanzepine, quetiapine, ziprasidone, sertindole, ketanserin, aripiprazole, and haloperidol, flupenthixol, thiroridazine, loxapine, fluspirilense, and sulpliride. The invention further provides methods for microvoltammetric imaging of changes in neurotransmitter concentrations in vivo and in real time comprising contacting the cell, cells, tissue, tissues, or organ of interest with a BRODERICK PROBE&reg; sensor, applying a potential to said BRODERICK PROBE&reg; sensor, and monitoring a temporally and spacially resolved recording using neuromolecular imaging (NMI) and electrochemical circuits such as, for example, voltammetry. In one embodiment of the invention, neuromolecular imaging may be performed before, during or after cocaine administration and/or cocaine-induced psychosis.

This application claims the benefit of U.S. Provisional Patent Application Ser. No. 60/526,833, filed on Dec. 4, 2003.

### STATEMENT OF GOVERNMENT SUPPORT

This invention was made, at least in part, with government support including Award No. SO 6 GM 08168 from the National Institutes Of Health (NIH/NIGMS). Therefore, the U.S. government has certain rights in the invention.

### FIELD OF THE INVENTION

The present invention provides methods of treating cocaine-induced psychosis by administering an atypical antipsychotic compound in an amount sufficient to increase serotonin concentration in the nucleus accumbens of a mammal. According to the invention, atypical antipsychotic compounds include, without limitation, clozapine, risperidone, olanzepine, quetiapine, ziprasidone, sertindole, ketanserin, aripiprazole, and haloperidol, flupenthixol, thioridazine; loxapine, fluspirilene, and sulpiride. The present invention further provides methods for real time neuromolecular imaging (NMI) of changes in neurotransmitter concentrations in vivo. In one embodiment of the invention, neuromolecular imaging may be performed before, during or after cocaine administration and/or cocaine-induced psychosis.

### BACKGROUND OF THE INVENTION

Addiction and Psychoses: We continue to strategize possible pharmacotherapies for cocaine-induced psychosis by studying the effects of a variety of typical and atypical antipsychotic medications on cocaine-induced neurochemistry and psychomotor stimulant behavior. The hypothesis derives from similarities between schizophrenic and cocaine psychosis, similarities which are being reported in the clinical literature at an alarming rate (Sherer et al., 1988; Brady et al., 1991; Mitchell and Vierkant, 1991; Nambudin and Young, 1991; Satel and Edell, 1991; Mendoza et al., 1992; Miller et al., 1992; Taylor and Staby, 1992; Tueth, 1993; Lysaker et al., 1994; Rosse et al., 1994; Rosenthal and Miner, 1997; Serper et al., 1999; Harris and Batki, 2000). Cocaine psychosis is a major psychopathology (Satel et al., 1991) and hyperfunction of DA-ergic systems is a critical element in cocaine-induced psychosis (Lieberman et al., 1990). Too, what complicates the situation further, are data which show that about 50% of the patients who suffer from schizophrenia have also been substance abusers at some time during their illness. Actually, schizophrenic patients are reported to feel the need to alleviate their psychosis by self-treating with reinforcing drugs (Mueser et al., 1995; Buckley, 1998).

Psychomotor Stimulant Animal Model of Addiction and Psychosis: One way to strategize such treatments for cocaine addiction and psychosis is to reverse certain elements of the disorders by utilizing laboratory studies in animals (McKinney, 1989). The strategy is reasonable especially since data from animal studies of stimulant psychosis and human schizophrenic psychosis share the same neurochemical and behavioral manifestations (Wise and Bozarth, 1987; Gawin et al., 1989; Margolin et al., 1995; Wise, 1995). Also, because cocaine is self-administered, universally across species (Risner and Jones, 1980; Fischman, 1984), it is highly likely that similar universal underlying reward mechanisms and mechanisms of consequent adverse symptomatology are similar from species to species.

The animal model of psychomotor stimulant behavior for cocaine addiction and psychosis has been validated by using this model to correlate antipsychotic medications and DA-ergic neuroanatomic pathways, e.g., but not exclusively, typical antipsychotics act through DA within nigrostriatal pathways and atypical antipsychotics act through DA and 5-HT within mesolimbic and mesocorticolimbic pathways (Cools and van Rossum, 1970; Costall and Naylor, 1973; Kelly et al, 1975; Pijnenburg et al., 1975; Wise and Bozarth, 1987; Broderick, 2001). Human data support these animal data. (Gawin and Kleber, 1986a; Gawin and Kleber, 1986b; Gawin et al., 1989; Meltzer, 1989). Hence, the psychomotor stimulant animal model has become an accepted model to study cocaine psychosis, albeit limited to certain aspects of the disease. An accepted neuroanatomic site for testing reversal of positive symptoms of psychosis is within NAcc, mesolimbic nerve terminals (Weinberger et al., 1992).

Cocaine, Monoamine Transporters and Release Mechanisms: Cocaine has a high affinity for monoamine transporters, and via these transporters, reuptake of monoamines into presynaptic nerve terminals is inhibited (Koe, 1976; Izenwasser et al., 1990); interestingly, certain subjective reward and jittery effects from cocaine have recently been associated with these monoamine transporters (Hall et al., 2002). In addition, cocaine has been shown to be dependent on stimulated release mechanisms (Ng et al., 1991) and on basal release mechanism by using the DA impulse flow inhibitor, gamma butyrolactone (γBL) (Broderick, 1991b). Although cocaine is not a direct receptor acting agonist, enhancement of DA neurotransmission may also be provided adjunctly through indirect activation of DA receptors, i.e., D₁ and D₂ (Spealman et al., 1992; Wise, 1995).

Cocaine, Monoamine Concentrations and Reward Mechanisms: Cocaine increases DA concentrations in mesolimbic neuronal circuits and the evidence suggests that the mechanism underlying cocaine's rewarding effect involves hyperfunction of the mesolimbic DA system, particularly in A₁₀ nerve terminals, NAcc (Hernandez and Hoebel, 1988; Kalivas and Nemeroff, 1988; Broderick, 1991a; Broderick, 1992b; Brown et al., 1991) and in A₁₀ somatodendrites, ventral tegmental area (VTA) (Einhorn et al., 1988; Bradberry and) Roth, 1989; Kalivas and Duffy, 1990; Broderick, 1992a; Kalivas, 1993). There is a general consensus from both clinical and preclinical studies that DA mediates the rewarding effects of cocaine (de Wit and Wise, 1977; Roberts et al., 1977; Roberts and Koob, 1982; Wise and Bozarth, 1987; Gawin et al., 1989; Wise and Rompre, 1989; Lieberman et al., 1990; Wise, 1995; Tsibulsky et al., 1998).

Cocaine increases 5-HT concentrations in A₁₀ terminals, NAcc, after single administration (Bradberry et al., 1993; Broderick et al., 1993). Sensitized 5-HT efflux in NAcc occurs after repeated cocaine administration (Parsons and Justice, 1993) and cocaine-increases 5-HT release induced by electrical stimulation of A₁₀ neurons, in vitro (Chen and Reith, 1993). Importantly, when 5-HT concentrations are deficient, such as in the Fawn-Hooded laboratory rat, cocaine-induced increases in 5-HT release are attenuated (Hope et al., 1995). Consistent with increased concentrations of 5-HT after cocaine, cocaine inhibits 5-HT reuptake in vitro (Ross and Renyi, 1969) and more recent studies have shown that cocaine inhibits 5-HT reuptake specifically in NAcc (Galloway, 1990). Also consistent with increased concentrations of 5-HT after cocaine, cocaine represses impulse frequency rates in vivo and in vitro in 5-HT somatodendrites, DR (Cunningham and Lakoski, 1988; Pan and Williams, 1989). Furthermore, 5-HT-cocaine interactions have been associated with transporter mechanisms (Reith et al., 1983; Carroll et al., 1993; Hall et al, 2002).

Nonetheless, a precise association between 5-HT and brain reward remains to be determined. Dietary I-tryptophan, a 5-HT precursor, and fluoxetine, a 5-HT reuptake inhibitor, have been reported to reduce cocaine self administration (Carroll et al., 1990a,b; McGregor et al., 1993; Peltier et al., 1994) and depletion of forebrain 5-HT with parachlorophenylalanine (PCPA) facilitates cocaine self-administration (Loh and Roberts, 1990; Richardson and Roberts, 1991). However, there are studies which are discrepant from these previous studies (Porrino et al., 1989). Also, self-stimulation studies, using 5-HT_{2A} antagonists and mixed DA₂/5-HT_{2A} antagonists, have suggested no involvement between 5-HT_{2A}, brain stimulation and cocaine stimulation reward (Ramana and Desiraju, 1989; Frank et al., 1995; Moser et al., 1995; Tsibulsky et al., 1998). Particularly relevant is a possible interpretation from the latter studies, that atypical antipsychotics may not affect the regulation of positive affect while still blocking neurochemical and behavioral effects of cocaine which may lead to psychosis.

Cocaine, Monoamines and Psychomotor Stimulant Behavior: Intra-NAcc infusions of cocaine mimics the hyperlocomotor effects of cocaine (Delfs et al., 1990) and the DA mesolimbic pathway has been directly implicated in the behavioral effects of cocaine (Kalivas and Nemeroff, 1988). Manipulations of 5-HT modulate the locomotor stimulant effects of cocaine (Walsh and Cunningham, 1997). Cocaine increases 5-HT in DA mesolimbic pathways simultaneously with increased locomotion, but the temporal pattern is disrupted compared with 5-HT increases with exploratory activity (Broderick, 2001). Specific 5-HT receptor mediation has been shown to correlate with open-field locomotion, e.g., local application of 5-HT and 5-HT_{1A} agonist, 8-OH-DPAT into median raphe nuclei causes hyperactivity (Hillegaart et al., 1989) and 8-OH-DPAT, has been shown to upmodulate cocaine-induced psychostimulant behavior (De La Garza and Cunningham, 2000). Specific 5-HT_{2A} and 5-HT_{2C} receptor mediation has been shown to correlate with cocaine-induced hyperactivity (McMahon and Cunningham, 2001; McMahon et al., 2001; Filip and Cunningham, 2002).

Monoamine Interactions, Basis for Cocaine Mechanisms: Turnover of DA is altered in NAcc when the 5-HT somatodendrites, DR, are electrolytically lesioned and these interactions modulate locomotion (Costall et al., 1976; Herve et al., 1979; Costall et al., 1990). Somatodendrites for DA, VTA, contain dense networks of 5-HT axonal varicosities. (Steinbusch, 1981; Herve et al., 1987; Van Bockstaele et al., 1994; Broderick and Phelix, 1997) and axons in NAcc core and shell exhibit overlapping of tyrosine hydroxylase (TH) and 5-HT (Van Bockstaele and Pickel, 1993, Phelix and Broderick, 1995).

Pharmacotherapies for Psychoses: Pharmacotherapies for cocaine psychosis are virtually non-existent. Thus far, clinicians are relying for therapy on antipsychotic medications and reasonably so because, as mentioned previously, neurochemical and behavioral similarities exist between schizophrenic and cocaine psychosis. Our main focus, then, is also in treatment strategies for cocaine based on antipsychotics, particularly in the area of atypical antipsychotic medications due to their dual interactions on dopamine (DA) and serotonin (5-HT) in DA neuronal pathways primarily in the mesolimbic/mesocorticolimbic A₁₀ neuronal circuitry; this now well-known 5-HT₂/DA₂ receptor affinity in the A₁₀ circuit, helps to alleviate both positive and negative symptoms of psychosis in addition to being mood enhancers (Meltzer, 1989; Meltzer, 1992). The leading hypothesis for the mechanism of action of these newer generation, atypical antipsychotic agents, is the presence of a high 5-HT-to-DA receptor blockade ratio in mesolimbic and mesocorticolimbic neural circuits. When 5-HT-ergic activity is blocked as is the case with many atypical antipsychotics, DA inhibition of DA release is also blocked, consequently, increasing presynaptic DA release and balancing DA blockade at postsynaptic receptor sites. The final result is less risk for EPS (Glazer, 2000).

Clozapine: Clozapine is considered to be the prototype of the atypical antipsychotics as it was the first to be recognized as having few if any EPS, not causing tardive dyskinesia or Parkinson's side effects including dystonia (Lieberman et al., 1989; Parsa et al., 1991). It is interesting that clozapine is not generally a first line defense drug against schizophrenia, but clozapine is especially effective for treating drug-resistant schizophrenia, when typical antipsychotics have failed the patient (Kane et al., 1988; Ranjan and Meltzer, 1996). Clozapine does not produce catalepsy (Kruzich and See, 2000). On the other hand, it is well known that clozapine may produce agranulocytosis in 0.0.5-2% of patients; blood serum levels must be monitored weekly for the first six months. Sedation and weight gain are limiting factors in clozapine treatment (Stahl, 2000).

The varied effects of clozapine may come about because the receptor binding profile for clozapine is complex. Clozapine binds to the following receptors: 5-HT_{1A}, 5-HT_{2A}, 5-HT_{2C}, 5-HT₃, 5-HT₆, 5-HT₇, DA₁, DA₂, DA₃, DA₄, M₁, H₁, α₁ and α₂ (Schotte et al., 1993; Brunello et al., 1995; Pere, 1995; Schotte et al., 1996; Stahl, 2000). Clozapine has high affinity for 5HT_{2A} receptors and low affinity for DA₂ receptors (Meltzer, 1991; Meltzer and Nash, 1991; Meltzer, 1999; Meltzer et al., 1992). Of the occupancy ratios for atypical antipsychotic medication, clozapine has the lowest occupancy for DA₂ receptors (Meltzer et al., 1992; Kapur and Remington, 2001).

Clozapine/Cocaine: Clozapine is an excellent candidate to test reversal of cocaine's effect, not only because of low DA receptor occupancy which is thought to reduce EPS, but also because clozapine is prescribed for cocaine addiction with reasonable success, i.e., clozapine pretreatment diminishes subjective responses to cocaine, including expected high and rush responses (Farren et al., 2000). In another study, pretreatment with clozapine has been shown to alleviate cocaine abuse in more than 85% of active substance (cocaine) abusers (Zimmet et al., 2000).

### SUMMARY OF THE INVENTION

The present invention provides methods of treating cocaine-induced psychosis by administering an atypical antipsychotic compound in an amount sufficient to increase serotonin concentration in the nucleus accumbens of a mammal. According to the invention, atypical antipsychotic compounds include, without limitation, clozapine, risperidone, olanzepine, quetiapine, ziprasidone, sertindole, ketanserin, aripiprazole, and haloperidol, flupenthixol, thioridazine, loxapine, fluspirilene, and sulpiride. The present invention further provides methods of increasing the level of serotonin in the nucleus accumbens of a mammal comprising administering an atypical antipsychotic compound in an amount sufficient to increase serotonin concentration in the nucleus accumbens. The invention further provides methods for microvoltammetric imaging of changes in neurotransmitter concentrations in vivo and in real time comprising contacting the cell, cells, tissue, tissues, or organ of interest with a BRODERICK PROBE® sensor, applying a potential to said BRODERICK PROBE® sensor; and monitoring a temporally and spacially resolved recording using neuromolecular imaging (NMI) and electrochemical circuits such as, for example, voltammetry. In one embodiment of the invention, neuromolecular imaging may be performed before, during or after cocaine administration and/or cocaine-induced psychosis.

### BRIEF DESCRIPTION OF THE DRAWINGS

FIGS. 1A-1C. FIG. 1A: Day 1: Acute Studies: Effects of cocaine (Coc) and clozapine/cocaine (Cloz/Coc) combination on DA release in NAcc of freely moving and behaving, Sprague-Dawley Rattus Norvegicus. Line graphs show Acute responses for DA. Axes: x axis, Pre-Drug denotes time for baseline values for DA, Post Drug denotes time after drug injection(s); y axis represents % change in DA produced by drug injection(s). Cocaine (N=5) increased DA (p<0.001). Results from administration of clozapine/cocaine combined (N=6), show that clozapine blocked cocaine-induced DA during the 2 hr time course study (p<0.001).

FIG. 1B: Day 1: Acute Studies: Effects of cocaine (Coc) and clozapine/cocaine (Cloz/Coc) combination on 5-HT release in NAcc of freely moving and behaving, Sprague-Dawley Rattus Norvegicus. Line graphs show Acute responses for 5-HT. Axes: x axis, Pre-Drug denotes time for baseline values for 5-HT, Post Drug denotes time after drug injection(s); y axis represents % change in 5-HT produced by drug injection(s). Cocaine (N=5) increased 5-HT (p<0.001). Results from administration of clozapine/cocaine combined (N=6), show that clozapine blocked cocaine-induced 5-HT release during the 2 hr time course study (p<0.001).

FIG. 1C: Day 1: Acute Studies: Effects of cocaine (Coc) and clozapine/cocaine (Cloz/Coc) combination on Locomotion (Ambulations) in freely moving and behaving, Sprague-Dawley Rattus Norvegicus. Line graphs show Acute responses for Locomotion. Axes: x axis, Pre-Drug denotes time for baseline values for locomotion, Post Drug denotes time after drug injection(s); y axis represents change in frequency for locomotion produced by drug injection(s). Cocaine (N=5) increased locomotion over baseline (p<0.001). Results from clozapine/cocaine combined (N=6), show that clozapine blocked cocaine-induced locomotion during the 2 hr time course study (p<0.001).

FIGS. 2A-2C. FIG. 2A: Day 2: Subacute Studies: Effects of cocaine (Coc) and clozapine/cocaine (Cloz/Coc) combination on DA release in NAcc of freely moving and behaving, Sprague-Dawley Rattus Norvegicus. Line graphs show Subacute responses for DA. Axes: x axis, Pre-Drug denotes time for baseline values for DA from Day 1 studies (Acute), Post Drug denotes time for Day 2, DA values (Subacute), when no further drug was administered to drug groups (same animal control); y axis, % change in DA compared with baseline. On Day 2, in the cocaine group (N=5), DA decreased from baseline (p<0.001), likely withdrawal related. Similarly, in the clozapine/cocaine group (N=6), DA decreased from baseline (p<0.001). There was no significant difference in DA effects between Subacute cocaine and clozapine/cocaine groups (p>0.05). Thus, the data suggest that DA-related cocaine withdrawal responses, Subacutely, may not be affected by clozapine.

FIG. 2B: Day 2: Subacute Studies: Effects of cocaine (Coc) and clozapine/cocaine (Cloz/Coc) combination on 5-HT release in NAcc of freely moving and behaving, Sprague-Dawley Rattus Norvegicus. Line graphs show Subacute responses for 5-HT. Axes: x axis, Pre-Drug denotes time for baseline values for 5-HT from Day 1 studies (Acute), Post Drug denotes time for Day 2, 5-HT values (Subacute), when no further drug was administered to drug groups (same animal control); y axis, % change in 5-HT from baseline. On Day 2, in the cocaine group (N=5), 5-HT decreased from baseline at the 15 min mark and during the second part of the 1 hr time course (p<0.05), likely reflecting 5-HT-related cocaine withdrawal effects. However, in the clozapine/cocaine group (N=6), 5-HT increased above baseline (p<0.001). There was a significant difference between Subacute cocaine versus clozapine/cocaine groups (p<0.001). The data suggest that clozapine, which has a longer pharmacokinetic half-life than does cocaine, may have reversed the 5-HT-related withdrawal effects of cocaine.

FIG. 2C: Day 2: Subacute Studies: Effects of cocaine (Coc) and clozapine/cocaine (Cloz/Coc) combination on Locomotion (Ambulation) in freely moving and behaving, Sprague-Dawley Rattus Norvegicus. Line graphs show Subacute responses for Locomotion. Axes: x axis, Pre-Drug denotes time for baseline values for locomotion from Day 1 studies (Acute), Post Drug denotes time for Day 2, locomotor values (Subacute), when no further drug was administered to drug groups (same animal control); y axis represents change in frequency of locomotor counts compared with baseline. On Day 2, in the cocaine group (N=5), locomotion was increased over baseline values (p<0.05). In the clozapine/cocaine group (N=6), locomotor counts showed no change from (Day 1) baseline (p>0.05). There was a significant difference between Subacute values in the cocaine group versus the clozapine/cocaine group (p<0.05). Due to clozapine's longer-lived pharmacokinetic properties, clozapine-induced sedation may be the mechanism for continued, diminished locomotion during Subacute (Day 2) studies.

FIGS. 3A-3N summarize the results of a comparison study of clozapine and ketanserin, description of a recording, an example of a BRODERICK PROBE® sensor, and a schematic of the technology of neuromolecular imaging. FIG. 3A Introduction. FIG. 3B methods. FIG. 3C drugs. FIG. 3D an example of the BRODERICK PROBE®. FIG. 3E microvoltammetry diagram. FIG. 3F typical voltammogram. FIGS. 3G-3L results. FIG. 3M conclusions: acute studies. FIG. 3N conclusions: subacute studies.

FIGS. 4A-4D. FIG. 4A: Day 1: Acute Studies: Effects of risperidone, cocaine and risperidone/cocaine combination on DA release in NAcc of freely moving and behaving, Sprague-Dawley Rattus Norvegicus. Line graphs show Acute responses for DA. Axes: x axis, Pre-Drug denotes time for baseline values for DA, Post Drug denotes time after drug injection(s); y axis represents % change in DA produced by drug injection(s). Risperidone (N=5) and cocaine (N=4) increased DA [(p<0.001), (p<0.01) respectively]. Results from administration of risperidone/cocaine combined (N=4), show that risperidone blocked cocaine-induced DA during the first hr of study (p<0.01).

FIG. 4B: Day 1: Acute Studies: Effects of risperidone, cocaine and risperidone/cocaine combination on 5-HT release in NAcc of freely moving and behaving, Sprague-Dawley Rattus Norvegicus. Line graphs show Acute responses for 5-HT. Axes: x axis, Pre-Drug denotes time for baseline values for 5-HT, Post Drug denotes time after drug injection(s); y axis represents % change in 5-HT produced by drug injection(s). Risperidone (N=5) and cocaine (N=4) increased 5-HT (p<0.001). Results from administration of risperidone/cocaine combined (N=4), show that risperidone blocked cocaine-induced 5-HT release during the 2 hrs of study (P<0.001).

FIG. 4C: Day 1: Acute Studies: Effects of risperidone, cocaine and risperidone/cocaine combination on Locomotion (Ambulations) in freely moving and behaving, Sprague-Dawley Rattus Norvegicus. Line graphs show Acute responses for Locomotion. Axes: x axis, Pre-Drug denotes time for baseline values for locomotion, Post Drug denotes time after drug injection(s); y axis represents change in frequency for locomotion produced by drug injection(s). Risperidone (N=5) did not affect locomotion (p>0.05) and cocaine (N=4) increased locomotion over baseline (p<0.001). Results from risperidone/cocaine combined (N=4), show that risperidone blocked cocaine-induced locomotion during the 2 hr period of study (p<0.001).

FIG. 4D: Day 1: Acute Studies: Effects of risperidone, cocaine and risperidone/cocaine combination on Stereotypy (Fine Movements: Sniffing and Grooming) in freely moving and behaving, Sprague-Dawley Rattus Norvegicus. Line graphs show Acute responses for Stereotypy. Axes: x axis, Pre-Drug denotes time for baseline values for stereotypy, Post Drug denotes time after drug injection(s); y axis represents change in stereotypic counts produced by drug injection(s). Risperidone (N=5) did not affect stereotypy (p>0.05) and cocaine (N=4) increased stereotypy over baseline (p<0.001). Results from risperidone/cocaine combined (N=4), show that risperidone blocked cocaine-induced stereotypy during the 2 hr period of study (p<0.001).

FIGS. 5A-5D. FIG. 5A: Day 2: Subacute Studies: Effects of risperidone, cocaine and risperidone/cocaine combination on DA release in NAcc of freely moving and behaving, Sprague-Dawley Rattus Norvegicus. Line graphs show Subacute responses for DA. Axes: x axis, Pre-Drug denotes time for baseline values for DA from Day 1 studies (Acute), Post Drug denotes time for Day 2, DA values (Subacute), when no further drug was administered to drug groups (same animal control); y axis, % change in DA compared with baseline. On Day 2, in the risperidone group (N=5), DA was not different from baseline (p>0.05). However, in the cocaine group (N=4), DA decreased from baseline, (p<0.001), likely withdrawal related. In the risperidone/cocaine group (N=4), DA-related cocaine withdrawal effects were reversed (p<0.001), possibly via risperidone.

FIG. 5B: Day 2: Subacute Studies: Effects of risperidone, cocaine and risperidone/cocaine combination on 5-HT release in NAcc of freely moving and behaving, Sprague-Dawley Rattus Norvegicus. Line graphs show Subacute responses for 5-HT. Axes: x axis, Pre-Drug denotes time for baseline values for 5-HT from Day 1 studies (Acute), Post Drug denotes time for Day 2, 5-HT values (Subacute), when no further drug was administered to drug groups (same animal control); y axis, % change in 5-HT from baseline. On Day 2, in the risperidone group (N=5), 5-HT was increased (p<0.001). However, 5-HT decreased from baseline at specific time points (15, 20, 50 and 55 min (p<0.05) in the cocaine group (N=4), reflecting 5-HT-related cocaine withdrawal effects. In the risperidone/cocaine group (N=4), 5-HT returned to baseline, suggesting a reversal of 5-HT-related cocaine withdrawal effects possibly by risperidone, but, there was no significant difference between Subacute cocaine versus risperidone/cocaine groups (p>0.05).

FIG. 5C: Day 2: Subacute Studies: Effects of risperidone, cocaine and risperidone/cocaine combination on Locomotion (Ambulation) in freely moving and behaving, Sprague-Dawley Rattus Norvegicus. Line graphs show Subacute responses for Locomotion. Axes: x axis, Pre-Drug denotes time for baseline values for locomotion from Day 1 studies (Acute), Post Drug denotes time for Day 2, locomotor values (Subacute), when no further drug was administered to drug groups (same animal control); y axis represents change in frequency of locomotor counts compared with baseline. On Day 2, in the risperidone group (N=5), locomotion was not affected (p>0.05). In the cocaine group (N=4), locomotion was also not significantly affected (p>0.05). In the risperidone/cocaine group (N=4), locomotor counts were still statistically insignificant when compared with baseline (p>0.05). It is noteworthy that in all three groups, locomotor counts did increase above baseline in the first half hr of study.

FIG. 5D: Day 2: Subacute Studies: Effects of risperidone, cocaine and risperidone/cocaine combination on Stereotypy (Fine Movements, Sniffing and Grooming) in freely moving and behaving Sprague-Dawley Rattus Norvegicus. Line graphs show Subacute responses for Stereotypy. Axes: x axis, Pre-Drug denotes time for baseline values for stereotypy from Day 1 studies (Acute), Post Drug denotes time for Day 2, stereotypy values (Subacute), when no further drug was administered to drug groups (same animal control); y axis represents change in frequency of stereotypic counts compared with baseline. On Day 2, in the risperidone group (N=5), stereotypy was increased above baseline (p<0.05). In the cocaine group (N=4), stereotypy was not significantly affected compared to baseline (p>0.05), although stereotypy increased above baseline during the study. Similarly to the data from the cocaine group, in the risperidone/cocaine group (N=4), stereotypy was also not altered compared to baseline (p>0.05), but stereotypic counts did increase above baseline during the study.

### DETAILED DESCRIPTION OF THE INVENTION

Clozapine Studies

Summary

There is an increasing awareness that a psychosis, similar to that of schizophrenic psychosis, can be derived from cocaine addiction. Thus, the prototypical atypical antipsychotic medication, clozapine, a 5-HT₂/DA₂ antagonist, was studied for its effects on cocaine-induced dopamine (DA) and serotonin (5-HT) release in Nucleus Accumbens (NAcc) of behaving Sprague Dawley laboratory rats with In Vivo Microvoltammetry, while animals' locomotor (forward ambulations), an A₁₀ behavior, was monitored at the same time with infrared photobeams. Release mechanisms for monoamines, were determined by using a depolarization blocker, gamma-butyrolactone (γBL). BRODERICK PROBE® microelectrodes selectively detected release of DA and 5-HT within seconds and sequentially in A₁₀ nerve terminals, NAcc. Acute and Subacute studies were performed for each treatment group. Acute studies are defined as single injection of drug(s) after a stable baseline of each monoamine and locomotor behavior has been achieved. Subacute studies are defined as 24 hr follow-up studies on each monoamine and locomotor behavior, in the same animal at which time, no further drug was administered.

Results showed that (1) Acute administration of Cocaine (10 mg/kg, i.p.) (N=5) significantly increased both DA and 5-HT release above baseline (p<0.001) while locomotion was also significantly increased above baseline (p<0.001). In Subacute studies, DA release decreased significantly below baseline (p<0.001) and significant decreases in 5-HT release occurred at the 15 min mark and at each time point during the second part of the hr (p<0.05); the maximum decrease in 5-HT was 40% below baseline. Locomotor behavior, on the other hand, increased significantly above baseline (p<0.05). (2) Acute administration of Clozapine/Cocaine (20 mg/kg i.p. and 10 mg/kg i.p., respectively) (N=6) produced a significant block of the cocaine-induced increase in DA (p<0.001) and 5-HT release (p<0.001). Cocaine-induced locomotion was blocked simultaneously with each monoamine by clozapine as well (p<0.001). In Subacute studies, DA release continued to be blocked presumably via clozapine by exhibiting a statistically significant decrease (p<0.001), but 5-HT release increased significantly (p<0.001), while cocaine-induced locomotor activity also continued to be antagonized by clozapine, i.e., locomotor activity exhibited no difference from baseline (p>0.05).

In summary, Acute studies (a) support previous data from this laboratory and others that cocaine acts as a stimulant on the monoamines, DA and 5-HT and on locomotor behavior as well and (b) show that clozapine, 5-HT₂/DA₂ antagonist, blocked enhanced DA, 5-HT and psychomotor stimulant behavior induced by cocaine. Subacute studies (a) suggest that withdrawal responses occurred in the cocaine group, based on recorded deficiencies in monoamine neurotransmitters, (b) show that withdrawal effects in the cocaine group likely presynaptic, were distinguished from locomotor behavior, classically known to be mediated postsynaptically and finally, (c) suggest that clozapine, with longer-lived pharmacokinetic properties, reversed 5-HT cocaine-related withdrawal effects, but was unable to reverse DA cocaine-related withdrawal responses. Taken together with data from this laboratory, in which the 5-HT_{2A/2C} antagonist, ketanserin, affected cocaine neurochemistry in much the same way as did clozapine, a mediation by either separate or combined 5-HT_{2A/2C} receptors for these clozapine/cocaine interactions, is suggested. Further studies, designed to tease out the responses of selective 5-HT_{2A} and 5-HT_{2C} receptor compounds to cocaine and clozapine/cocaine, are underway.

Methods

### Drugs

Clozapine was obtained from Sigma/Aldrich, St. Louis, Mo., dissolved in distilled water, and the pH of the solution was adjusted to 2.7 with citric acid powder. Cocaine was obtained from Sigma Aldrich, St. Louis, Mo. and dissolved in distilled water.

### Animals

Animals were purchased from Charles River Laboratories, Kingston, N.Y. and were housed in our animal care facilities for one to two weeks before surgery was performed. The Animal Care Facility operates under the auspices of the CUNY, City College Institutional Animal Care and Use Committee (IACUC) in compliance with National Institute of Health (NIH) guidelines. The weight range for the animals, at the time of the studies, was 350-475 g. Animals were group housed before surgery, individually housed after surgery and fed Purina Rat Chow and water ad libitum. A twelve hr dark-light cycle was maintained both in the housing of the animals and throughout the experimental studies.

### Surgical Procedures and Implantation of Microelectrodes

Protocols Follow Paradigm Described in (Broderick et al., 2003).

Each animal was anesthetized with pentobarbital Na, (50 mg/kg i.p., (dilute (6%) solution)) and stereotaxically implanted with a BRODERICK PROBE® indicator microelectrode in ventrolateral (vl) NAcc (AP=+2.6, ML=+2.5, DV=−7.3) (Pellegrino et al., 1979). The stereotaxic equipment was purchased from David Kopf Instruments, Tujunga, Calif. A Ag/AgCl reference electrode was placed in contact with dura, 7 mm anteriorally and contralaterally to the indicator microelectrode. A stainless steel auxiliary microelectrode was placed in contact with dura. BRODERICK PROBE® microelectrodes were manufactured on site. The BRODERICK PROBE® electrode is described in the following United States and international patents and applications: U.S. Pat. No. 4,883,057; U.S. Pat. No. 5,433,710; WO 91/02485; EP 0487647 B1; HK 1007350; CA 2,063,607; U.S. application Ser. No. 10/118,571, and U.S. Provisional Patent Application No. 60/526,833, which are herein incorporated by reference in their entirety.

Animals' body temperature was continuously monitored with a rectal probe and thermometer (Fisher Sci., Fadem, N.J.). Body temperature was maintained at 37.5° C.±0.5° C. with an aquamatic K module heating pad (Amer. Hosp. Supply, Edison, N.J.). Booster injections of pentobarbital Na were administered once after the first two hrs of surgery (0.10 cc) and once every subsequent hr (0.05 cc) to maintain an adequate level of anesthesia throughout surgery. The total time for surgery was three to four hrs. The indicator, reference, and auxiliary microelectrodes were held in place with dental acrylic (Jet Line, Lang Dental Inc., CA). Animals recovered in a bedded Plexiglas cage (dimensions: 12 in (width), 12 in (depth), 18 in (height)) after surgery and before the experimental studies began, with food and water ad libitum. The animals were treated with physiological saline (0.5 cc) immediately and for one to two days after surgery as needed. The antibiotic, chloramphenicol (50 mg/kg i.p.) was administered if needed.

In vivo microvoltammetric studies on conscious Sprague-Dawley laboratory rats were begun nine to fifteen days after the aseptic surgical operations were performed. On each experimental day, the animal was placed in a Plexiglas-copper faradaic chamber. The three-microelectrode assembly, enclosed within the animal's prosthetic acrylic cap, was connected to a CV37 detector by means of a mercury commutator (Br. Res. Instr., Princeton, N.J.), a flexible cable, and a mating connector (BJM Electronics, Staten Island, N.Y.). The CV37 detector was electrically connected to a Minigard surge suppresser (Jefferson Electric, Magnetek, N.Y.) which was then connected to an electrical ground in isolation. Stable electrochemical signals for DA and 5-HT were evident before either (i) clozapine (20 mg/kg i.p.), (ii) cocaine (10 mg/kg i.p.) or (iii) combination of clozapine and cocaine (20 mg/kg i.p. and 10 mg/kg i.p., respectively) were administered. Each animal was used as its own control. Changes in synaptic concentrations of DA and 5-HT are presented as percent change (% of control) in order to minimize normal between-animal variations. Currents recorded were in the order of magnitude of pA or nA. In vivo microvoltammetric scans were recorded in sec and repeated every five min for a period of 2 hrs before each treatment and a period of two hrs after each treatment.

### In Vivo Microvoltammetry

### Technology

In Vivo Microvoltammetry with a semidifferential (semiderivative) circuit was used; a clear separation of the monoamine neurotransmitters, DA and 5-HT was achieved. Dopamine and 5-HT were detected within sec, in separate signals. Oxidation peak potentials (half-wave potentials) of +0.14±0.015V and +0.29±0.015V were characteristic for DA and 5-HT. Detailed methodology is published (Broderick, 1988; Broderick, 1989; Broderick, 1990; Broderick, 1991b; Broderick et al., 1993; Broderick, 1999; Broderick et al., 2000; Broderick, 2001; Broderick, 2002; Broderick and Pacia, 2003). The electrochemical signal for DA was detected without interference at the same oxidation potential, from 3-4-dihydroxyphenylacetic acid (DOPAC), homovanillic acid (HVA) and ascorbic acid (AA). Indeed, clear and separated signals are routinely achieved with BRODERICK PROBE® microelectrodes for AA, HVA and DOPAC. The electrochemical signal for 5-HT was detected without interference at the same oxidation potential, from the 5-HT metabolite, 5-hydroxyindoleacetic acid (5-HIAA) and uric acid (UA). Potentials were applied with a CV37 detector (BAS, West Lafayette, Ind.). Potentials were applied from −0.2V to +0.4V with respect to a Ag/AgCl (1M NaCl) electrode, at a scan rate of 10 mV/sec at time constants of 5 and 1 tau. One scan was completed in 60 sec. Non-faradaic charging current was eliminated in the first 25 sec. The neurotransmitters, DA and 5-HT, were detected in approximately 10-15 sec. and 10-12 sec, respectively with BRODERICK PROBE® stearic acid microelectrodes and 10-12 sec and 8-10 sec, respectively, with BRODERICK PROBE® lauric acid microelectrodes. The coulombic efficiency for the detection of 5-HT was two to three fold greater than that for DA (Broderick, 1987).

Calibration curves were determined experimentally, in vitro, in a freshly prepared deoxygenated physiological saline-phosphate buffer solution (0.01 M, pH=7.4). We use ultra pure Nitrogen (N₂) (T. W. Smith Corp., Bklyn, N.Y.) to deoxygenate the buffer solution. Solutions of DA and 5-HT (99% purity, Sigma Aldrich, St. Louis, Mo.) as well as metabolites of the monoamines, were aliquoted into the buffer and the peak height of the electrochemical signals were correlated with specific nM and uM concentrations. Calibration studies were also performed in freshly prepared deoxygenated buffer solutions containing phosphotidylethanolamine (PEA), combined with bovine serum albumin (BSA) (Sigma Aldrich, St. Louis, Mo.), a solution which closely mimics brain constituents. These studies showed that lipid constituents of brain and not proteins, amplify the detection sensitivity of the indicator microelectrodes, supporting previous data which show that lipids amplify electrochemical signals detected by BRODERICK PROBE® microelectrodes; the phenomenon is termed The Lipid Amplification Number (LAN) (Broderick, 1999; Broderick et al., 2000). Surface Enhanced Raman Spectroscopy (SERS) and Raman Resonance (RR) techniques have correlated our findings on signal amplification by lipids (Foucault et al., 2002). Detection limits for basal synaptic concentrations of DA and 5-HT in NAcc were 12 nM and 2 nM respectively. Placement of indicator microelectrodes in NAcc of each animal, was confirmed by the potassium ferrocyamide blue dot method, using a current of 50 mA for period of 30 sec. Virtually no damage to brain tissue occurred. Recording characteristics of microelectrodes were stable.

### Behavior

Locomotor activity (ambulation) was monitored with infrared photobeams at the same time as DA and 5-HT release in NAcc was detected with BRODERICK PROBES® in conjunction with In Vivo Microvoltammetry. The chamber was faradaic, covered with copper to refract possible electrical artifacts (dimensions: 24 in (width) by 18 in (depth) by 23.5 in (height)). A 16 by 16 array of these infrared photobeams, were held in place by an aluminum frame which was situated ¾ in above the Plexiglas floor of the chamber to detect locomotor activity. Photobeams were sampled by a Pentium computer to define the x-y position of the animal within a 1.5 in resolution every 100 msec. When an x-y position was calculated, it was used to define the frequency of locomotor activity in counts. The locomotor activity system is a modified version of an Activity Pattern Monitor (APM) (San Diego Instruments, San Diego, Calif.). Behavioral data is presented in absolute frequency, i.e., number of counts recorded.

The first hr pre-drug allowed exploratory behavior. Exploratory behavior is defined as open-field behavior of ambulations (forward locomotion) wherein animals respond to the stimuli of a novel environment with high frequency of behavioral counts. The second hr pre-drug allowed the animal to become habituated before treatment. Habituation behavior is defined as a behavioral state in which behavior exhibits reduced responses to novel stimuli; animals cease exploring or searching in their novel environment and maintain a steady-state response to novel stimuli.

In the Acute studies, each drug was administered at the end of the habituation period. Baseline (control) values were taken every five min for the last thirty min of the habituation period at which time drug injection(s) took place. Electrochemical recordings for DA and 5-HT release in NAcc, were continued for two hrs; at the same time, locomotor behavior continued to be monitored and recorded with infrared photobeams. At the end of the 2 hr drug(s) study, animals were then placed back in their home cages.

In the Subacute studies, which took place twenty-four hrs later, the animals were again placed in the faradaic behavioral chamber and no further drug was administered. Recordings of separate electrochemical signals for DA and 5-HT release in NAcc, were taken for one hr; at the same time, locomotor behavior was monitored and recorded with infrared photobeams.

### Data Analysis

Neurochemical and behavioral data, derived from the last thirty minutes of the habituation period, provided the baseline data. Statistically significant differences between baseline and post-drug injection(s) for (1) DA, (2) 5-HT and (3) locomotor behavior were determined by subjecting the data to One Way Analysis of Variance (ANOVA) (tested at criteria p=0.05), with subsequent application of the post hoc test, Tukey's Multiple Comparison Test. Where appropriate, data points in the time course were subjected to 95% Confidence Limits (C.L.).

### Results

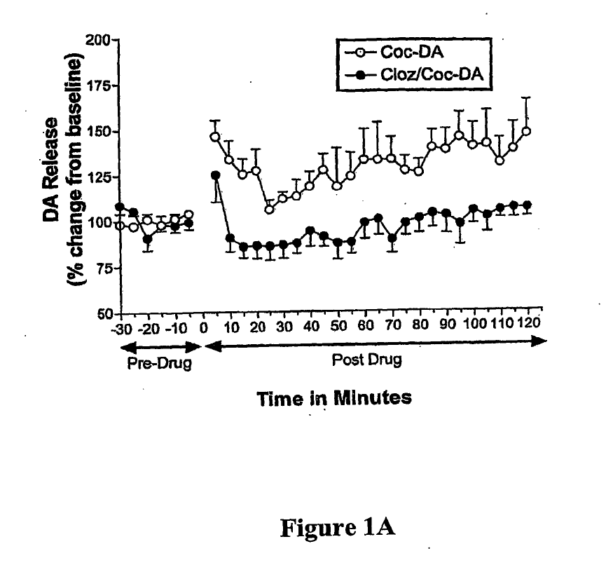

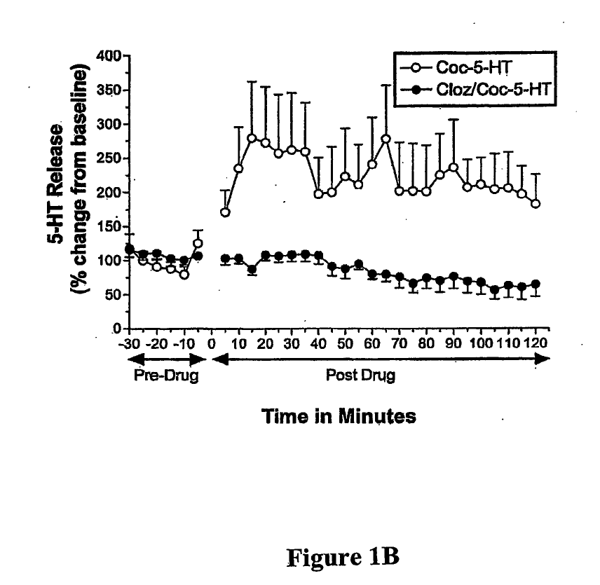

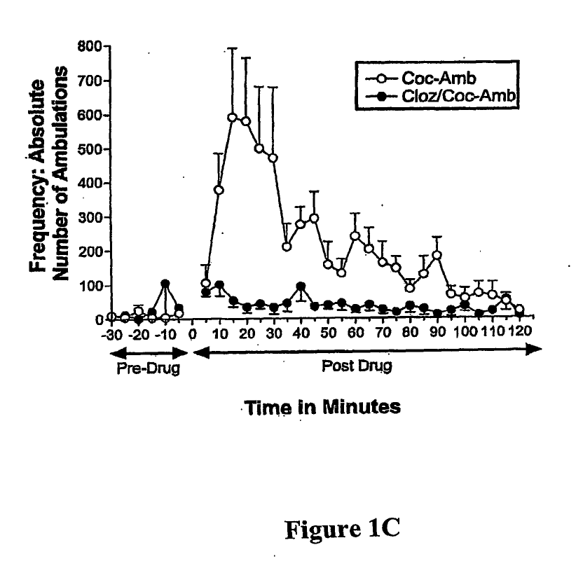

FIG. 1A: Day 1: Acute Studies: Effects of Cocaine or Clozapine/Cocaine Combination on DA Release in NAcc:

Cocaine: (open circles) Cocaine significantly increased DA release over baseline (habituation) values (One Way ANOVA: p<0.0001; F=51.17; df=3.56). Post hoc analysis showed that significant differences between pre-cocaine (baseline) and post-cocaine (same animal control) occurred as well (Tukey's Multiple Comparison Test: p<0.001, q=9.498).

Clozapine/Cocaine: (closed circles) Clozapine significantly blocked the effects of cocaine on DA release (One Way ANOVA: p<0.0001; F=51.17; df=3.56). Post hoc analysis showed significant differences between cocaine and clozapine/cocaine groups (Tukey's Multiple Comparison Test: p<0.001, q=16.43).

FIG. 1B: Day 1: Acute Studies: Effects of Cocaine and Clozapine/Cocaine Combination on 5-HT Release in NAcc:

Cocaine: (open circles) Cocaine significantly increased 5-HT release over baseline (habituation) values (One Way ANOVA: p<0.0001; F=154.2; df=3.56). Post hoc analysis showed that significant differences between pre-cocaine (baseline) and post-cocaine (same animal control) occurred as well (Tukey's Multiple Comparison Test: p<0.001, q=16.19).

Clozapine/Cocaine: (closed circles) Clozapine significantly blocked the effects of cocaine on 5-HT release (One Way ANOVA: p<0.0001; F=154.2; df=3.56). Post hoc analysis showed significant differences between cocaine and clozapine/cocaine groups (Tukey's Multiple Comparison Test: p<0.001, q=28.98).

FIG. 1C: Day 1: Acute Studies: Effects of Cocaine and Clozapine/Cocaine Combination on Locomotion (Ambulations):

Cocaine: (open circles) Cocaine significantly increased locomotor activity (ambulations) over baseline (habituation) values (One Way ANOVA: p<0.0001; F=13.06; df=3.56). Post hoc analysis showed that significant differences between pre-cocaine (baseline) and post-cocaine (same animal control) occurred as well (Tukey's Multiple Comparison Test: p<0.01, q=5.688).

Clozapine/Cocaine: (closed circles) Clozapine significantly blocked the effects of cocaine on locomotor activity (One Way ANOVA: p<0.0001, F=13.06; df=3.56). Post hoc analysis showed significant differences between cocaine and clozapine/cocaine groups (Tukey's Multiple Comparison Test: p<0.001, q=7.784).

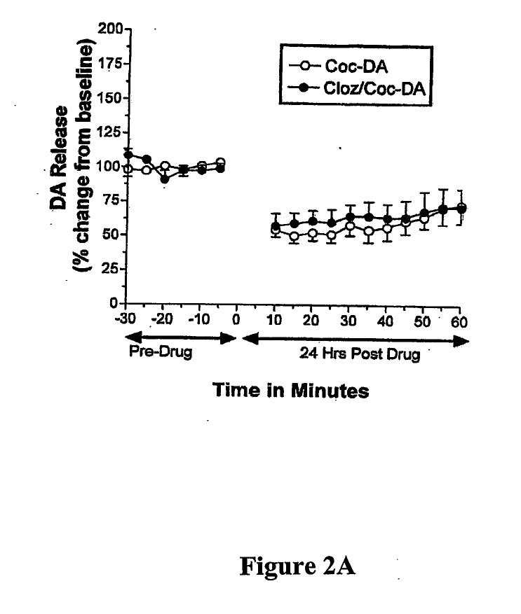

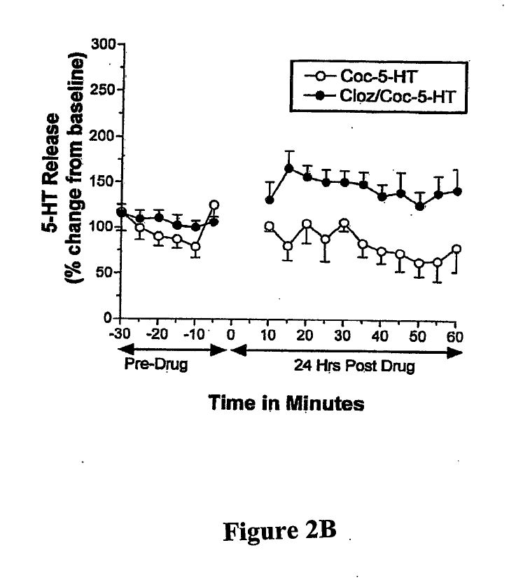

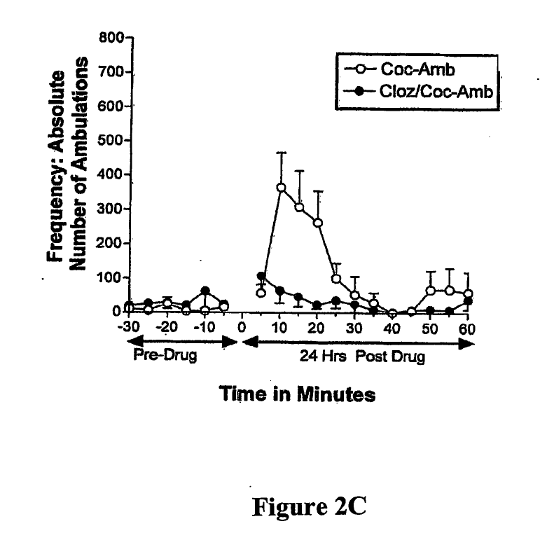

FIG. 2A: Day 2: Subacute Studies: Effects of Cocaine or Clozapine/Cocaine Combination on DA Release in NAcc:

Cocaine: (open circles) During the Subacute studies, when no further cocaine was administered, DA release in NAcc significantly decreased from baseline (habituation) values (from a significant increase) (One Way ANOVA: p<0.0001; F=106.3; df=3.30). Post hoc analysis showed that significant differences occurred between baseline (Day1) and (Day2) values (same animal control) (Tukey's Multiple Comparison Test: p<0.001, q=18.99). Compared to drug effect on Day 1, DA release was decreased dramatically by about 80% during the hr period of study.

Clozapine/Cocaine: (closed circles) During the Subacute studies, when no further drug(s) were administered, DA release in NAcc significantly decreased from baseline (habituation) values (One Way ANOVA: p<0.0001; F=106.3; df=3.30). Post hoc analysis showed that a significant difference occurred between baseline (Day1) and (Day2) values (same animal control). (Tukey's Multiple Comparison Test: p<0.001, q=16.48). A significant difference between cocaine and clozapine/cocaine (Day2) groups did not occur (Tukey's Multiple Comparison Test: p>0.05, q=16.48).

FIG. 2B: Day 2: Subacute Studies: Effects of Cocaine or Clozapine/Cocaine Combination on 5-HT Release in NAcc:

Cocaine: (open circles) During the Subacute studies, when no further cocaine was administered, 5-HT release in NAcc was decreased below baseline (Day1) values compared to (Day2) values, at specific time points during the time course of the 1 hr study, i.e., at the 15 min mark and at each time point in second part of the hr (p<0.05); (One Way ANOVA: p<0.0001; F=38.99; df=3.30), although the post hoc analysis did not show statistical significance for the hr (Tukey's Multiple Comparison Test: p>0.05, q=3.225). Compared to drug effect on Day1, 5-HT release was decreased dramatically by approximately 150% in the second half hour of the study.

Clozapine/Cocaine: (closed circles) During the Subacute studies, when no further drug(s) were administered, 5-HT release in NAcc was significantly increased above (Day1) baseline values (One Way ANOVA: p<0.0001; F=38.99; df=3.30), Post hoc analysis showed significant differences between baseline (Day1) and (Day2) values (same animal control) (Tukey's Multiple Comparison Test: p<0.001, q=7.704). Significant differences occurred between cocaine (Day2) and clozapine/cocaine groups (Day2) (Tukey's Multiple Comparison Test: p<0.001, q=14.90).

FIG. 2C: Day 2: Subacute Studies: Effects of cocaine or clozapine/cocaine combination on Locomotion (Ambulations):

Cocaine: (open circles) During the Subacute studies, when no further cocaine was administered, locomotor activity on (Day2) was significantly increased over baseline (Day1) values (One Way ANOVA: p<0.0186; F=3.843; df=3.32). Post hoc analysis showed that significant differences occurred between baseline (Day1) and (Day2) values (same animal control) (Tukey's Multiple Comparison Test: p<0.05, q=3.925). Nonetheless, locomotor activity decreased by 250 counts when compared with (Day 1) effects of cocaine-induced psychomotor stimulation.

Clozapine/Cocaine: (closed circles) During the Subacute studies, when no further cocaine or clozapine were administered, locomotor activity remained significantly decreased (One Way ANOVA: p<0.0186; F=3.843; df=3.32). Post hoc analysis showed that no significant differences occurred between baseline (Day1) and (Day2) values (same animal control) (Tukey's Multiple Comparison Test: p>0.05, q=0.03647). Moreover, (Day2) cocaine and clozapine/cocaine groups did significantly differ (Tukey's Multiple Comparison Test: p<0.05, q=3.846).

In all groups, additional saline controls had no effect.

### Discussion

Cocaine, Monoamines and Psychomotor Stimulant Behavior: Acute Studies: We have extended our work from two recent articles on the effects of cocaine on DA and 5-HT release in NAcc of freely moving and behaving laboratory rats WHILE monitoring cocaine-induced psychomotor stimulant behavior simultaneously. Comparing the present data to the first of these recent papers (Broderick et al., 2003), we have simply added animals to our cocaine group. Comparing the present data to the second of these recent papers (Broderick and Piercey, 1998b), an entirely different group of animals was utilized. The results from all three studies from our laboratory were equivalent, i.e., increased DA, 5-HT release in NAcc occurred with increased psychomotor stimulant behavior (Broderick, 2001; Broderick et al., 2003).

Cocaine, Monoamines and Psychomotor Stimulant Behavior: Subacute Studies: Cocaine studies in the Subacute group were also extended by increasing the number of animals above what was used in our previous studies (Broderick et al., 2003); again, the results were equivalent. When no further drug was administered, there was a significant decrease in DA release in NAcc and significant decreases in 5-HT release during specific points in the time course data. The data are in agreement with several reports (Parsons et al., 1995; Parsons et al., 1996; Broderick et al., 1997). In addition, the data agree with a previous report showing long-lasting effects after a single moderate dose of cocaine (Zahniser et al., 1988). Behavioral activity maintained an increase at the same time that accumbens DA and 5-HT release were decreased, thereby suggesting dissociative function between behavior classically known to be mediated via DA₂ postsynaptically and monoamine release and reuptake inhibitory mechanisms, presynaptically.

The subacute data suggest that these monoamine deficiencies may be associated with symptoms of withdrawal and the data agree with clinical reports of DA-ergic systems and craving (Dackis and Gold, 1985; Gawin and Kleber, 1986a; Gawin and Kleber, 1986b; Lieberman et al., 1990; Margolin et al., 1995). Neuroadaptation may be occurring as reported in animal studies (Koob and Nestler, 1997; Broderick, 2001) because neither the short pharmacokinetic half-life of cocaine nor that of its metabolites, provides a rational explanation (Misra et al., 1974a,b; Nayak et al., 1976; Mets et al., 1999; Sun and Lau, 2001). Of course, another plausible explanation though, is one provided by others, that transient compensatory changes take place a day after cocaine cessation (Zahniser et al., 1988).

Cocaine, Monoamine Interactions, Possible Mechanisms: Classical cocaine mechanisms point to a postsynaptic DA₂ release with additional DA release derived presynaptically from DA somatodendrites, VTA. Current thinking on the mechanism of action of cocaine points to a DA/5-HT interaction in DA mesolimbic circuits. A postsynaptic 5-HT-ergic upmodulation of DA in NAcc has been implicated; the 5-HT_{2A/2C} receptor has been shown to upmodulate DA release in NAcc after intermittent cocaine (Yan et al., 2000) and endogenously, as well (Yan, 2000). Local application (infusion) of [(+/−)-2,5-dimethoxy-4-iodoamphetamine hydrochloride] (DOI), a 5-HT_{2A/2C} agonist, was infused into NAcc to increase DA which was subsequently blocked by ketanserin, a 5-HT_{2A/2C} antagonist. Furthermore, infusion of DOI in NAcc was antagonized by the selective 5-HT_{2C/2B} receptor antagonist, SB 206553, but not by the selective 5-HT_{2A} antagonist, SR 46349B (Lucas and Spampinato, 2000), suggesting that DA increases in NAcc after infusion of DOI may be due to a mediation by the 5-HT_{2C} receptor.

However, prematurely pointing specifically to the 5-HT_{2C} receptor for cocaine's mechanism of action may present a limitation because local application (infusion into NAcc) of 5-HT_{2C} receptor agonists did not alter basal locomotor activity nor mimic the stimulus effects of cocaine (McMahon et al., 2001; Filip and Cunningham, 2002). Also, a recent report shows that a 5-HT_{2A} receptor mediation is prominent in blocking cocaine-induced locomotor activity (McMahon and Cunningham, 2001). Therefore, the mechanism of action of cocaine is probably dually directed, i.e., via classical DA₂ and currently explored 5-HT_{2A/2C}/DA₂ receptor circuits.

Although studies by Di Matteo et al., 1999, do not address cocaine, the data do importantly show the direct autoreceptor properties of selective 5-HT_{2C} receptor compounds viz a viz the effects of these compounds when locally applied by infusion. Thus, the selective 5-HT_{2C} antagonist, SB 242084, increased and the selective 5-HT_{2C} agonist, RO 60-0175, decreased DA release in NAcc (Di Matteo et al., 1999).

Clozapine/Cocaine: Acute Studies: Clozapine significantly reduced cocaine-induced increases in DA release in NAcc by an average of 40% in the first hr of study and 50% in the second hr of study. Simultaneously, clozapine significantly reduced cocaine-induced increases in 5-HT release in NAcc by an average of 138% in the first hr of study and average of 113% in the second hr of study. Also, at the same time, locomotor activity (ambulation counts) produced by cocaine, were reduced by an average of 500 counts in the first half hr and by an average of 150 counts in the next hr of study. Since these are the first studies of this kind ever performed, direct comparisons cannot be made. Nonetheless, the present studies are in general agreement with preclinical studies in which clozapine was shown to antagonize cocaine-induced place preference in animals (Kosten and Nestler, 1994) and to block reinforcement by intravenous cocaine in animals (Loh et al., 1992).

Clozapine/Cocaine: Possible Mechanisms of Action: Acute Studies: Without being bound by theory, the data suggest that increased 5-HT by cocaine leads to an increase in DA release perhaps via either a separate or combined 5-HT_{2A/2C} receptor mediation which is subsequently blocked by clozapine. This suggestion is made because of previous reports of the importance of 5-HT_{2A/2C} receptors either alone or combined in cocaine mechanisms (Yan, 2000; Yan et al., 2000; McMahon and Cunningham, 2001; Filip and Cunningham, 2002) and because clozapine is, in fact, the prototypical atypical 5-HT_{2A}/DA₂ receptor antagonist, although not exclusively bound to these two types of receptors. Clozapine has high antagonist affinity for the 5-HT_{2C} receptor and indeed, phosphoinositol inverse agonist activity at the 5-HT_{2C} receptor (Kuoppamaki et al., 1995; Herrick-Davis et al., 1999; Herrick-Davis et al., 2000) has been shown specifically in NAcc, in the action of clozapine (Di Matteo et al., 2002). Classical DA₂ postsynaptic antagonism of cocaine-induced psychomotor stimulant behavior by clozapine probably accounts, at least in part, for the complete blockade of motor activity observed. Interestingly, in the apomorphine-induced hypomotility test, clozapine did not antagonize this presynaptic response, unlike the DA_{2/3} antipsychotic agent, sulpiride (Robertson and MacDonald, 1986).

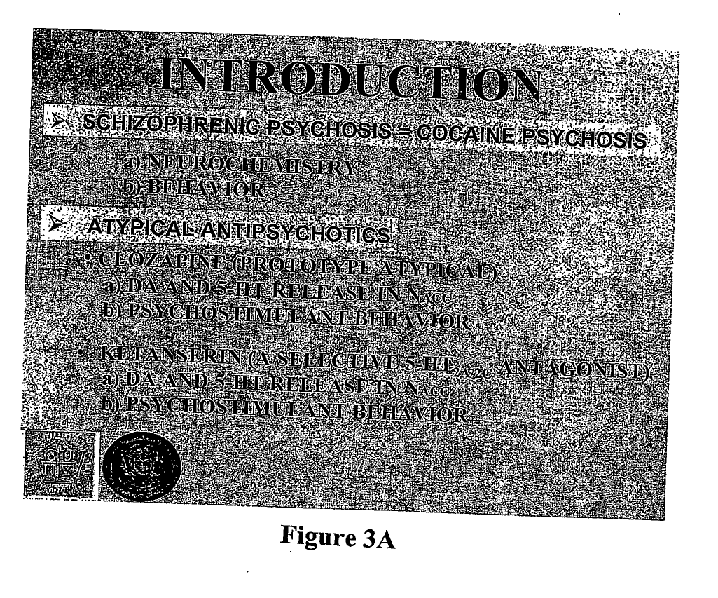

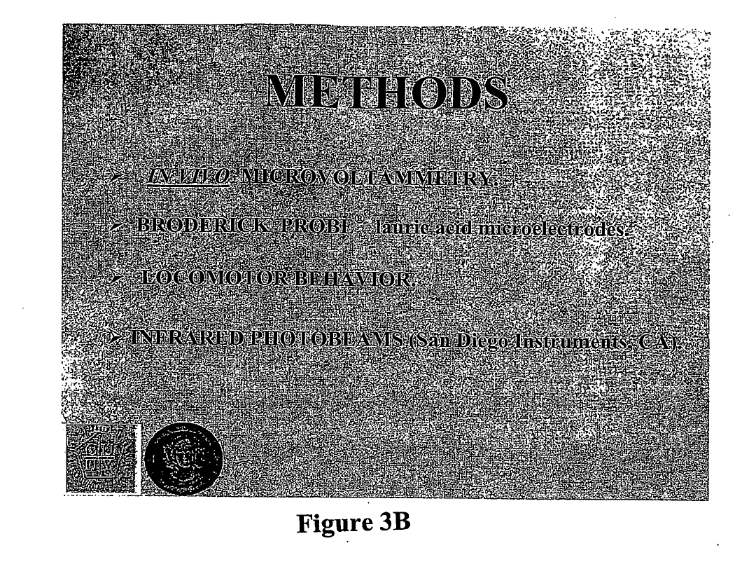

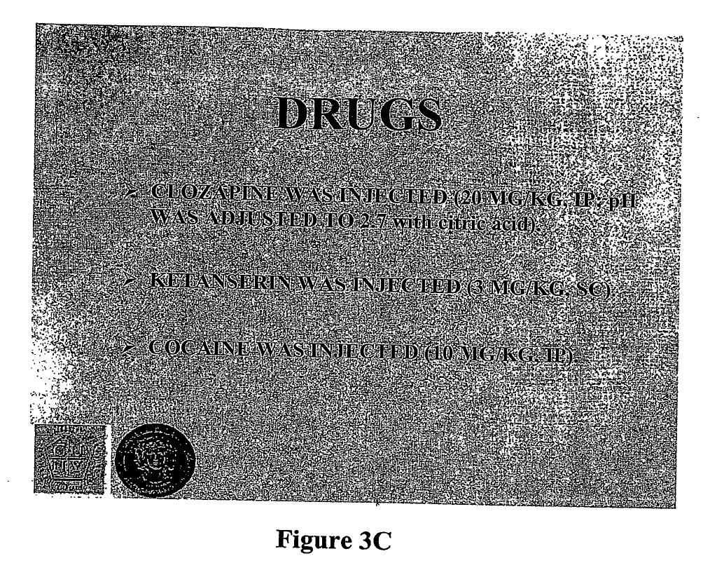

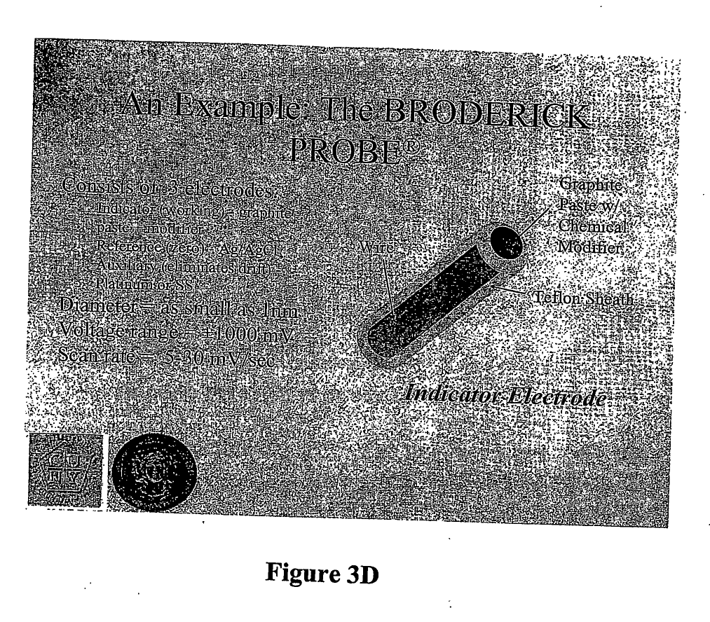

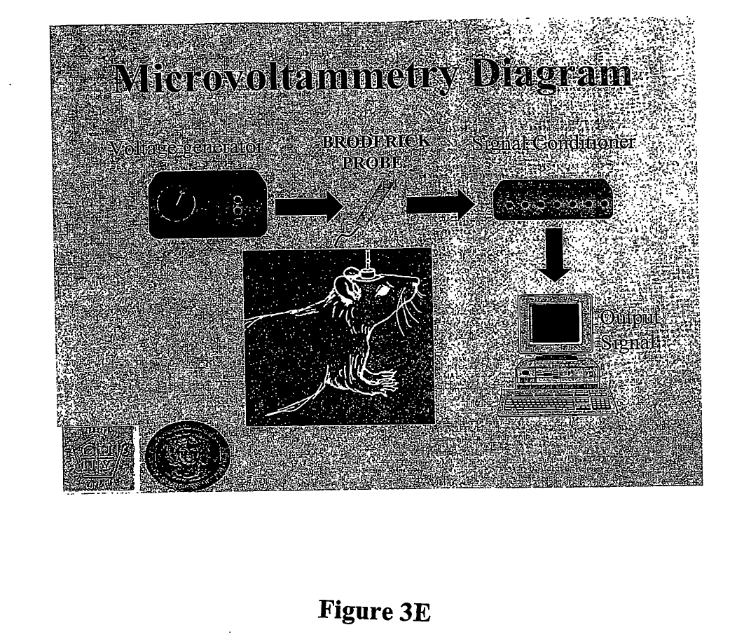

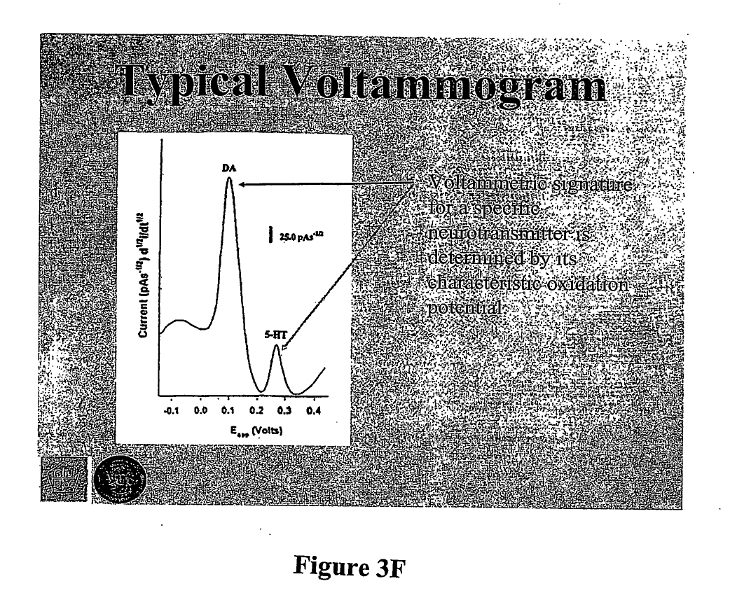

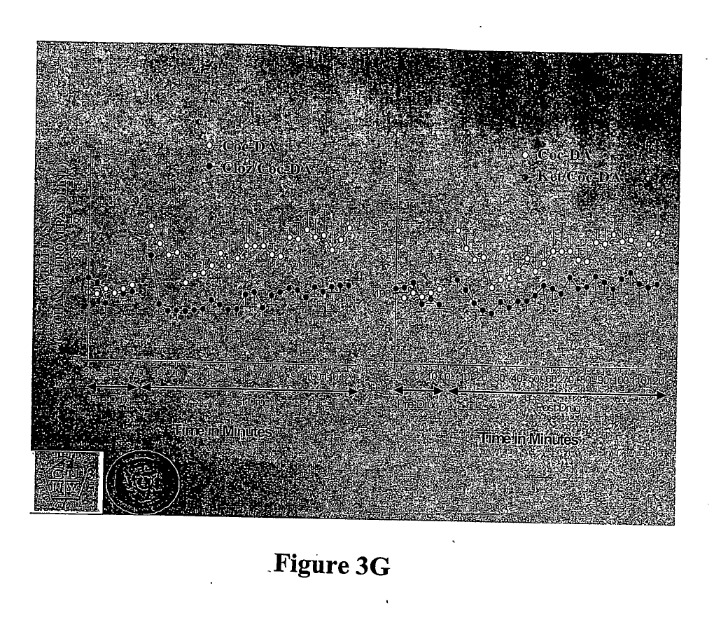

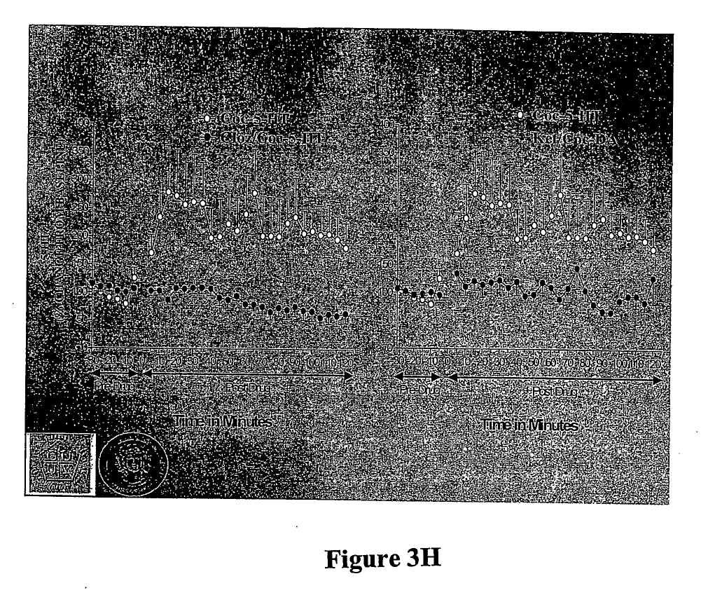

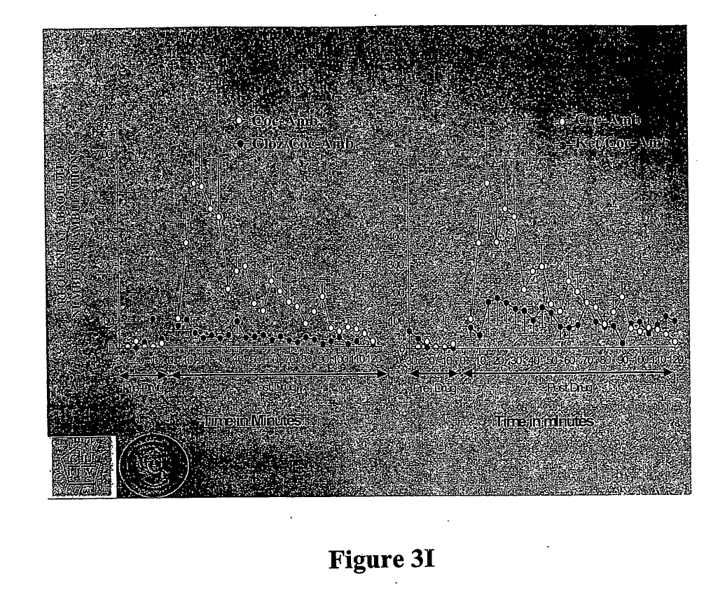

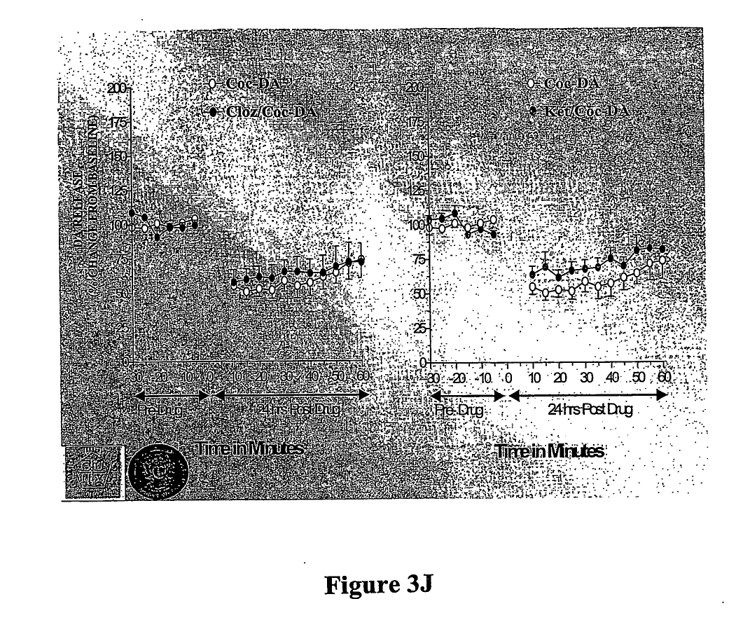

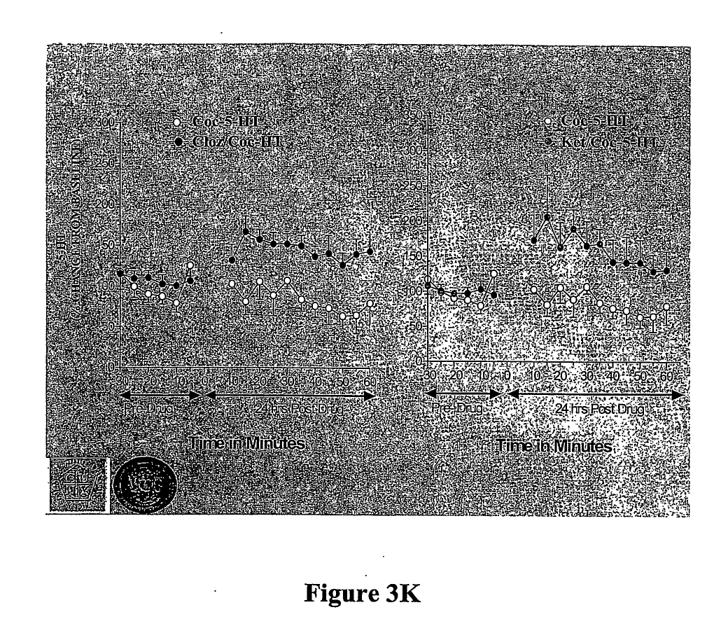

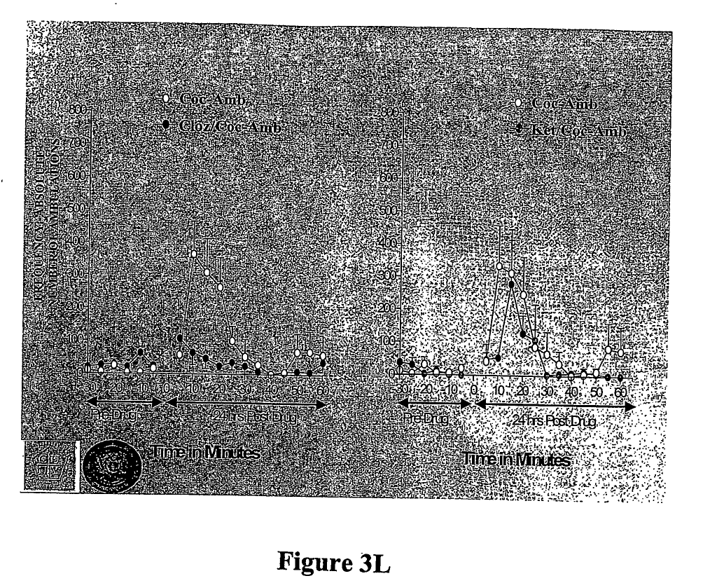

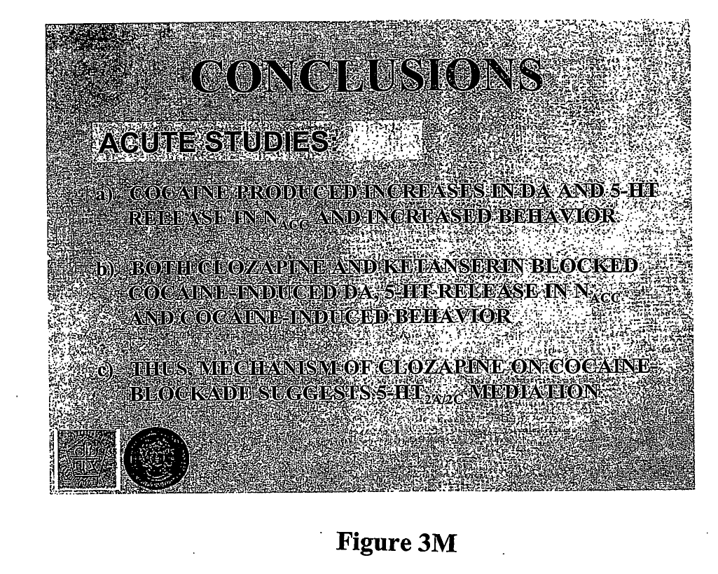

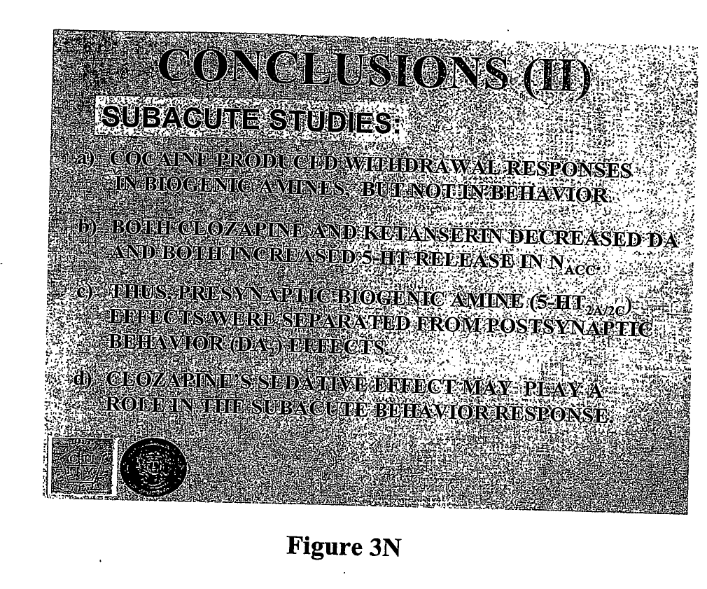

A mediation by 5-HT_{2A/2C} receptors in the mechanism of action for clozapine's blockade of cocaine, is also suggested. In Acute studies performed in this laboratory, we substituted (3 mg/kg s.c.) ketanserin, a 5-HT_{2A/2C} antagonist, for clozapine and the results were remarkably similar to clozapine in blocking cocaine-induced monoamine release and psychomotor stimulant behavior, although ketanserin blockade of cocaine was actually weaker than that of clozapine in all three parameters studied (Broderick et al., 2001). These results were published in Broderick, P A, Olabisi, O A, Rahni, D N, and Zhou, Y. Cocaine acts on accumbens mono-amines and locomoter behavior via 5-HT2A/2C receptor mechanism as shown by ketanserin: 24 h follow-up studies. Progress in Neuro-Psychopharmacology & Biological Psychiatry, vol. 28 (2004) 547-557, which is herein incorporated by reference in its entirety. This work was also the subject of a presentation given at the Society for Neuroscience meeting on Nov. 12, 2003 in New Orleans. The presentation is summarized in FIGS. 3A-3N.

Ketanserin and clozapine do not have similar receptor profiles, in general, but ketanserin is similar to clozapine in that both are direct receptor antagonists which bind with high affinity to 5-HT_{2A}, 5-HT_{2C}, adrenergic (α₁) and histamine (H₁) receptors; ketanserin does not bind to DA receptors (Lutje-Hulsik, 2002; Duffy et al., 2000). The strong α₁ influence is a concern, but there is good evidence that only 5-HT_{2A/2C} receptors are involved and α₁ adrenoreceptors are not involved in the mechanism of cocaine's stimulant activity (Filip et al., 2001). The high antagonist affinity for H₁ receptors on the part of both ketanserin and clozapine, is probably not a concern. Studies on H₁ and even H₂ promoter polymorphisms conclude that participation of these receptors has an unlikely influence in the clinical response to clozapine treatment (Mancama et al., 2002). Thus, 5-HT_{2A/2C} properties for clozapine are highly likely mechanisms for clozapine's antagonism of cocaine effects. These results were published in Broderick, P A, Olabisi, O A, Rahni, D N, and Zhou, Y. Cocaine acts on accumbens mono-amines and locomoter behavior via 5-HT_{2A/2C} receptor mechanism as shown by ketanserin: 24 h follow-up studies. Progress in Neuro-Psychopharmacology & Biological Psychiatry, vol. 28 (2004) 547-557, which is herein incorporated by reference in its entirety. This work was also the subject of a presentation given at the Society for Neuroscience meeting on Nov. 12, 2003 in New Orleans. The presentation is summarized in FIGS. 3A-3N.

It is interesting that studies on risperidone's effects on cocaine-induced stimulant monoamine neurochemistry and locomotor behavior, showed that risperidone completely blocked 5-HT release in NAcc and simultaneous locomotor activity but did not completely block DA release on NAcc. Indeed, risperidone significantly increased DA release in the second hr of the study, given the caveat that high dose risperidone (2 mg/kg s.c.) was tested (Broderick et al., 2003) (see below).

Clozapine/Cocaine: Subacute Studies: Results showed that DA release in NAcc, at this time, decreased by an average of 60% during the hr of study, WHILE 5-HT release increased by 50% above baseline for the hr of study and locomotor activity remained reduced by an average of 250 ambulatory counts in the first 20 min.

It is important to note that these long lasting effects of clozapine are supported by pharmacokinetics. The half-life of clozapine is 8 hrs at lower doses and 4-66 hrs at higher doses; the hydroxiated and N-oxide derivatives are reported to be inactive (Rx List Monographs, 2002). Interestingly, preliminary data from our laboratory have shown that significantly increased 5-HT release after combined clozapine/cocaine administration, does not begin to diminish until the fifth day of recovery after drug administration.

Clozapine/Cocaine: Possible Mechanisms of Action: Subacute Studies: The occurrence of increased 5-HT release in Subacute studies may be explained by clozapine's mechanism than by cocaine's mechanism. If we look at clozapine, 5-HT presynaptic autoreceptors, as studied in synaptosomes, may lend an explanatory note (Drescher and Hetey, 1988). Also, presumably by autoreceptors, clozapine increased DA efflux in NAcc (Volonte et al., 1997; Kuroki et al., 1999), DA and 5-HT release in NAcc in the behaving animal (Broderick et al., unpublished data; Ichikawa et al., 1998) and DA and 5-HT release in NAcc in the anesthetized animal (Broderick and Piercey, 1998a). Therefore, increased 5-HT release, as shown in these Subacute studies, may be mediated by inhibitory presynaptic autoreceptors. The explanation for decreased DA release is not apparent, unless this DA decrease is simply compensatory (Herve et al., 1979; Beart and McDonald, 1982).

Moreover, increased 5-HT release on the second day of study might possibly have been derived from increased cocaine serum levels due to the longer lasting effects of clozapine since a clinical study reported enhanced cocaine serum levels after administration of both clozapine and cocaine to cocaine addicts (Farren et al., 2000). It is noteworthy that increased 5-HT release on the second day of study, did not occur in a risperidone/cocaine group (Broderick et al., 2003).

Thus far, research in our laboratory suggests that clozapine's action on cocaine is mediated at least partly, via 5-HT_{2A/2C} receptors because the results presented here, from clozapine/cocaine combination studies, resemble those obtained during Subacute studies when substituting the 5-HT_{2A/2C} antagonist, ketanserin for clozapine. In Subacute studies with ketanserin/cocaine, when no further ketanserin or cocaine was administered, DA release in NAcc decreased and 5-HT increased to a statistically significant level, just as did results, Subacutely, in the clozapine/cocaine group.

There were some differences in monoamine reactions during the subacute studies between the ketanserin/cocaine group and the clozapine/cocaine group, e.g., the DA response was weaker and the 5-HT response was somewhat stronger. Notably, locomotor activity was significantly higher on the second day for the ketanserin/cocaine group viz-a-viz the clozapine/cocaine group. The clozapine/cocaine group continued to exhibit sedation, possibly through residual potent, muscarinic anticholinergic receptor mediation (Richelson and Souder, 2000; Broderick et al., 2001).

On the other hand, the atypical antipsychotic medication, the 5-HT_{2A}/DA₂ receptor antagonist, risperidone, did not exhibit subacute responses to cocaine as did clozapine. In fact, DA and 5-HT release returned to baseline and locomotor activity increased insignificantly above baseline (Broderick et al., 2003). It is noteworthy however, that studies with the high dose of risperidone (2 mg/kg, s.c.) were performed (Broderick et al., 2003). High dose risperidone exhibits more typical than atypical antipsychotic properties (Williams, 2001).

Not to be neglected, though, is a possible mediation by the 5-HT_{1A} receptor because clozapine exhibits moderate receptor binding for the 5-HT_{1A} receptor (Schotte et al., 1993; Sumiyoshi et al., 1995; Schotte et al., 1996). Importantly, the 5-HT_{1A} receptor has been shown clinically to mediate schizophrenic psychosis (Chou et al., 2003) and preclinically, to mediate the action of DA in NAcc (Ichikawa and Meltzer, 2000). Finally, α₁ antagonism has been shown to mediate inhibition of dorsal raphe (DR) firing by clozapine through 5-HT_{1A} receptors (Sprouse et al., 1999).

Conclusions: Acute studies showed that clozapine blocked accumbens DA, 5-HT and locomotor effects of cocaine. These studies are the first of their kind. The Subacute studies are also unique; the Subacute studies allowed us to study withdrawal effects of cocaine in addition to the unexpected long-lasting effects of clozapine/cocaine treatment on accumbens DA and 5-HT release in the freely moving and behaving animal. Enhanced 5-HT release may, help alleviate clinical depression associated with cocaine withdrawal (Price et al., 2001), although decreased DA release could be a disadvantage, possibly leading to craving (Dackis and Gold, 1985). Nonetheless, critical treatment strategies for cocaine addiction and psychosis could be derived from these results.

Risperidone Studies

Summary

In vivo microvoltammetry was used to detect dopamine (DA) and serotonin (5-HT) release from Nucleus Accumbens (NAcc) of freely moving, male, Sprague Dawley laboratory rats, while animals' locomotor (forward ambulations) and stereotypic behavior (fine movements of sniffing and grooming) were monitored at the same time with infrared photobeams. Monoamine release mechanisms were determined by using a depolarization blocker, gamma-butyrolactone (γBL). Miniature carbon sensors, BRODERICK PROBE® Lauric Acid Microelectrodes, smaller than a human hair, were used in conjunction with a semidifferential electrochemical circuit, to detect release of each monoamine in separate signals and within seconds. The purpose was to evaluate the neuropharmacology of the 5-HT₂/DA₂ antagonist, risperidone, in its current therapeutic role as an atypical antipsychotic medication, as well as in its potential role as pharmacotherapy for cocaine psychosis and withdrawal symptoms. Acute (single drug dose) and subacute studies (24 hr follow-up studies in the same animal, no drug administration) were performed for each treatment group. The hypothesis for the present studies is derived from a growing body of evidence that cocaine-induced psychosis and schizophrenic psychosis share similar neurochemical and behavioral manifestations.

Results showed (1) Acute administration of Risperidone (2 mg/kg, s.c.) significantly increased DA and 5-HT release in NAcc above baseline (habituation) values (p<0.001) while locomotion and stereotypy were virtually unaffected. In Subacute studies, DA release did not differ from baseline (p>0.05), whereas 5-HT release was significantly increased above baseline (p<0.001). Locomotion increased over baseline but not to a significant degree, while stereotypy was significantly increased above baseline (p<0.05). (2) Acute administration of Cocaine (10 mg/kg, i.p.) significantly increased both DA and 5-HT release above baseline (p<0.001) while locomotion and stereotypy were also significantly increased over baseline (p<0.001). In Subacute studies, DA decreased significantly below baseline (p<0.001) and significant decreases in 5-HT release occurred at 15, 20, 50 and 55 min (p<0.05); behavior increased above baseline, but did not reach a statistically significant degree. (3) Acute administration of Risperidone/Cocaine (2 mg/kg s.c. and 10 mg/kg i.p., respectively) showed a significant block of the cocaine-induced increase in DA release in the first hr (p<0.001) and 5-HT release in both hrs of study (p<0.001). Cocaine-induced locomotion and stereotypy were blocked simultaneously with the monoamines (p<0.001). In Subacute studies, DA and 5-HT release returned to baseline while locomotion and stereotypy increased insignificantly above baseline.

Thus, these studies (a) were able to tease out pharmacologically, critical differences between pre- vs. post-synaptic responses to drug treatment(s) and these differences may lead to more effective therapies for schizophrenic and/or cocaine psychosis. (b) Taken together with other data, these acute studies suggest that risperidone may possibly act via inhibition of presynaptic autoreceptors to produce the observed increases in accumbens DA and 5-HT release, whereas cocaine may be acting at least in part, via 5-HT-ergic modulation of DA postsynaptically. The subacute data suggest that pharmacokinetics may play a role in risperidone's action and neuroadaptation may play a role in the mechanism of action of cocaine. Finally, the ability of risperidone to block cocaine-induced psychostimulant neurochemistry and behavior during acute studies while diminishing the withdrawal symptoms of cocaine during subacute studies, suggest that risperidone may be a viable pharmacotherapy for cocaine psychosis and withdrawal.

Introduction

Schizophrenia: Yeats aptly said about schizophrenic psychosis, “Things fall apart; the center cannot hold; mere anarchy is loosed upon the world.” (Yeats, 1956). Schizophrenia is a major mental disorder in which the patient has difficulty in perceiving and then evaluating reality. Indeed, “schizophrenia” is believed to have earned its name because the patient experiences a “split” between thought and affect. Although multifaceted, schizophrenia is the prototypical psychosis; the classical hallmark features are divided into two main categories, positive and negative symptoms. Among the positive symptoms are auditory hallucinations, disorganized thoughts and speech, and paranoid delusions. The negative symptoms consist of amotivation, social isolation, poverty of speech and thought (APA, 2000). Simply stated, positive symptomatology has been said to reflect an excess of normal function and negative symptomatology seems to reflect a reduction in normal functions (Stahl, 2000). Although at first glance, the negative symptoms appear to be less disturbing than are positive symptoms in that negative symptoms may not interrupt so blatently the orderly course of life, negative symptoms can be and are debilitating.

Antipsychotic Medication: Moreover, negative symptoms are more difficult to reverse than are positive symptoms. In fact, conventional antipsychotic medications, such as haloperidol, a typical antipsychotic, do reverse positive symptoms but are not particularly effective in reversing the negative symptoms of psychosis (Carpenter et al., 1988). Atypical antipsychotic medications, such as risperidone, and clozapine, have been used with success for reversal of both the positive and negative symptoms of schizophrenic psychosis (Meltzer, 1992; Conloy and Mahmoud, 2001). Interestingly, typical antipsychotic medications and atypical antipsychotic medications exhibit some general differences as follow: (1) typical antipsychotic agents are DA antagonists which act on DA₂ receptors in the nigrostriatal neuronal circuit and induce adverse motor abnormalities, Extrapyramidal Symptoms (EPS), likely via this same receptor and DA pathway. Typicals are effective in reducing positive symptoms of psychosis, presumably also, via the DA₂ receptor and high DA receptor occupancy (Farde et al., 1988; Mukherjee et al., 2001) and typicals have little or no effect on 5-HT-ergic mechanisms (Broderick and Piercey, 1998a; Ichikawa et al., 1998). (2) Atypical antipsychotic drugs act primarily, but not exclusively, on 5-HT₂/DA₂ receptors in the mesocorticolimbic neuronal circuit to reduce negative as well as positive symptoms of psychosis while reducing the risk of EPS; it is thought that 5HT-ergic modulation of DA mediates reduction of EPS (Meltzer and Nash, 1991). (3) Moreover, from the aspect of mood, typical antipsychotic agents may produce anhedonia, i.e., a loss of “joie de vivre” (Blum et al., 1989), whereas the atypical antipsychotic medications have been reported to improve affective disorders, presumably via their 5-HT-ergic properties (Meltzer, 1989). Pharmacotherapies for schizophrenia have been reviewed (Seeman, 1987; Meltzer, 1991; King, 1998; Lieberman et al., 1998; Carlsson et al., 1999; Kane, 1999; Fink-Jensen, 2000; Kapur and Remington, 2001).

Another differentiation between the two antipsychotic types of medication, comes from pharmacological behavioral studies in animal models. Typicals exhibit inhibition of hyperactivity and stereotypy induced by DA-ergic drugs and in addition, induce catalepsy in a similar dose range; atypicals cause selective inhibition of hyperactivity without induction of stereotypy or catalepsy (Weiner et al., 2000; Wadenberg et al., 2001). Also, in animal models, an atypical antipsychotic agent e.g., perospirone, another novel 5-HT₂/DA₂ receptor antagonist, has been differentiated from typical antipsychotic agents on the basis of its preferential ability to induce Fos expression in rat forebrain in mesolimbic NAcc vs. nigrostriatal dorsolateral striatal terminal (Ishibashi et al, 1999).

Risperidone: Risperidone is one of these novel atypical antipsychotic medications with treatment efficacy for both negative and positive symptoms of schizophrenia and concomitantly, their use presents less risk of EPS (Marder and Meibach, 1994; Lemmens et al., 1999). In a group of schizophrenic patients with disturbing EPS from previous neuroleptic pharmacotherapy, risperidone was observed to have less liability for Parkinsons' symptoms than was the typical antipsychotic, haloperidol (Heck et al., 2000). Risperidone was developed following studies which showed that the negative symptoms of schizophrenia and EPS were improved when ritanserin, a selective antagonist at the structurally similar 5-HT₂ and 5-HT_{1C} receptors, was combined with haloperidol (Bersani et al., 1986).

A synthetic benzisoxazole derivative, risperidone is a highly selective 5-HT_{2A}/DA₂ antagonist with high affinity for these receptors as well as for α₁ and α₂ adrenergic receptors and the H₁ histamine receptor; low to moderate affinity is seen for the 5-HT_{2C}, 5-HT_{1A}, 5-HT_{1C}, and 5-HT_{1D} receptors (Janssen et al., 1988; Leysen et al., 1988; Leysen et al., 1992). Using constitutively active mutants of 5-HT_{2C} receptors, which are associated with high basal levels of intracellular inositol phosphate (IP), risperidone was found to have inverse agonist activity at human 5-HT_{2C} receptors (Herrick-Davis et al., 1999). A high affinity for the inverse agonist 5-HT_{2C} receptor was found in the rat choroid plexus (Canton et al., 1990; Kuoppamaki et al., 1995; Schotte et al., 1996). Risperidone binds with weak affinity to the DA₁ and haloperidol-sensitive sigma site, whereas no affinity for the cholinergic muscarinic or β₁ and β₂ adrenergic receptors has been reported (Keegan, 1994). Optimal dosing is important for risperidone therapy as DA₂ receptor affinity increases in the higher dose range, thereby increasing the risk of EPS (Williams, 2001). Therefore, the caveat exists that although risperidone is especially atypical at low doses, a more typical profile may be seen at the higher doses (Megens et al., 1992). Specifically, in the NAcc, in [3H] spiperone labeling studies, risperidone revealed biphasic inhibition curves indicating that 5-HT₂ receptor occupancy occurs (<0.04 mg/kg) and DA₂ receptor occupancy exhibits an ED₅₀ at (1.0 mg/kg) (Leysen et al. 1992).

Risperidone has some other favored uses, not only in schizophrenia but also in treating the depressive aspects of schizoaffective disorders (Myers and Thase, 2001), and in treating behavioral disturbances in children and adolescents with psychiatric dysfunction (Turgay et al., 2002). Behavioral locomotor and stereotypic disturbances in Lesch-Nyhan Syndrome have been decreased by risperidone (Allen et al., 1998)

Cocaine: Cocaine increases DA neurotransmission by inhibiting the DA reuptake transporter at the presynapse in DA nigrostriatal and mesolimbic neuronal pathways; increased DA neurotransmission is believed to occur via DA reuptake inhibition, enhanced release of DA or a combination of DA reuptake inhibitory and enhanced release mechanisms (de Wit and Wise, 1977; Church et al., 1987; Ritz et al., 1987; Bradberry and Roth, 1989; Hurd and Ungerstedt, 1989; Kalivas and Duffy, 1990; Broderick, 1991a; Broderick, 1991b; Broderick, 1992a; 1992b; Broderick et al., 1993). Increased DA neurotransmission in mesolimbic and mesocorticolimbic DA reward pathways (Wise and Rompre, 1989) is thought to emanate from Ventral Tegmental Area (VTA) (Roberts and Koob, 1982; Goeders and Smith, 1983; Evenden and Ryan, 1988; Einhorn et al., 1988; Kalivas, 1993; Broderick and Phelix, 1997).

The first studies that showed that cocaine increased 5-HT release in NAcc were performed in this laboratory (Broderick et al., 1993) and in Roth's laboratory at Yale (Bradberry et al., 1993). Serotonin has also been implicated in cocaine's electrophysiological, transporter, behavioral and reinforcing effects (Cunningham and Lakoski, 1988; Broderick, 1991b; Broderick, 1992a; Broderick, 1992b; Carroll et al., 1993; Broderick et al., 1997; Broderick, 2001, Hall et al., 2002). Other neurochemical and behavioral studies support these data (Parsons et al., 1996; Andrews and Lucki, 2001). The latter study reports that the effect of cocaine on 5-HT in DA somatodendritic autoreceptors was greater than that of DA (Andrews and Lucki, 2001). Moreover, 5-HT release in NAcc, VTA and striatum (Str) has been shown to increase rhythmically on line and in vivo with rhythmic movement during natural exploration whereas cocaine disrupted the balance between 5-HT release and natural exploratory rhythmic movement (Broderick, 2001). Interactions between 5-HT and DA are becoming more important in explaining cocaine's neurochemical and behavioral properties. A recent report suggests that cocaine increases DA probably via postsynaptically mediated 5-HT_{2C} receptor action. Adjunct mechanisms include additional DA release derived presynaptically from DA somatodendritic autoreceptors, VTA, via 5-HT_{2A} and feedback compensatory mechanisms (Filip and Cunningham, 2002).

Dopamine and 5-HT interactions are plausible in the mechanism of action of cocaine because both immunohistochemical studies (Steinbusch, 1981) and immunocytochemical studies (Broderick and Phelix, 1997) show that DA cell bodies, VTA, contain a dense network of 5-HT axonal varicosities. Neuroanatomic localization of tyrosine-hydroxylase containing (TH) and 5-HT-containing axons in NAcc, show a prominent overlap of DA and 5-HT axons in core and shell (Phelix and Broderick, 1995). Ultrastructural evidence from light and electron microscopy has shown that 5-HT neurons innervate DA neurons synaptically (Herve et al., 1987). A cellular basis is evidenced for the 5-HT excitation of DA neurons by the existence of asymmetric junctions formed by 5-HT labeled terminals in mesolimbic projections to NAcc (Van Bockstaele and Pickel, 1993; Van Bockstaele et al. 1994; Broderick and Phelix, 1997).

Cocaine Psychosis: Cocaine is a powerful reinforcer because the drug is a rewarding stimulant. Cocaine has even been reported to induce an orgasmic-type experience (Cohen, 1975; Seecof and Tennant, Jr., 1986). Cocaine's rewarding and reinforcing effects are so powerful that the cocaine addict risks becoming mentally ill with a syndrome known as “cocaine psychosis” (Brady et al., 1991). Prolonged cocaine psychosis, as any psychotic event, is a major psychopathology (Satel et al., 1991). In fact, emergency cases of patients diagnosed with cocaine-induced psychosis are being considered by some, as alarming (Mendoza et al., 1992; Taylor and Staby, 1992; Tueth, 1993) and clinical reports of paranoid psychosis induced by cocaine are becoming common (Sherer et al., 1988; Satel and Edell; 1991), even in the elderly (Namboudin and Young, 1991). Cocaine “paranoia” has been likened to schizophrenic “paranoia” (Rosse et al., 1994). Finally, data from Single Photon Emission Computerized Tomography (SPECT) studies show that cocaine induced changes in cerebral blood flow are similar to those seen in patients diagnosed with schizophrenic psychosis (Miller et al., 1992).

Interestingly, animal models of cocaine psychosis share similar neurochemical and behavioral manifestations with human schizophrenic psychosis. Very early on, the animal model of psychomotor stimulant behavior was validated. Psychostimulant behavior was shown to be dependent on DA-ergic nigrostriatal neuronal pathways in animals (Cools and Van Rossum, 1970; Costall and Naylor, 1973; Wise and Bozarth, 1987; cf. Broderick, 2001 for review). Dopamine antagonists block psychostimulant behavior (Pijnenburg et al., 1975). Supporting these animal data, typical antipsychotic medications, which act through DA-ergic nigrostriatal neuronal pathways reduced psychotic symptoms in humans (Gawin and Kleber, 1986a; Gawin and Kleber, 1986b). Also supporting these animal data, atypical antipsychotic medications, which act through DA-ergic mesolimbic/mesocorticolimbic neuronal pathways, reduce psychotic symptoms in humans (Meltzer, 1989). Hence, psychostimulant-induced neurochemistry and behavior has become an accepted animal model of psychosis, albeit limited to certain aspects of the disease.

Risperidone/Cocaine: It is noteworthy that clinical effects of risperidone on cocaine have met with some success, e.g., on substance abusing schizophrenic patients (Tsuang et al., 2002) on craving (Smelson et al., 2002) on euphoric effects of cocaine (Newton et al., 2001) on cocaine dependence (Grabowski et al., 2000), on cue-elicited craving (Smelson et al., 1997), thereby adding significance to the present data. Preclinically, cocaine cueing properties (Van Campenhout et al., 1999) and 5-HT₂/DA₂ antagonism of brain stimulation reward (Tsibulsky et al., 1998), have also been elegantly reported, but to date, to the authors' knowledge, this is the first paper to present the effects of risperidone in the psychostimulant animal model of psychosis.

This cutting edge technology, in vivo microvoltammetry with miniature carbon sensors, BRODERICK PROBES® (Broderick, 1999) is particularly suitable for studies of neurochemistry because the technology provides excellent spatial and temporal resolution as well as selectivity for separate neurotransmitters. The technology allows a high degree of accuracy because it allows direct electrochemical detection of neurotransmitters within a specific neuroanatomic site. Also, since few electrical connections are used for direct in vivo detection of neurotransmitters, it avoids bulky inflow and outflow perfusate tubings, apparently required for other methods. Too, subacute (24 hr follow-up) studies allow withdrawal symptoms and possible reversal of withdrawal symptoms to be studied in the same animal reliably and accurately as glial formation around the microelectrodes is virtually non-existent.

Methods

### Drugs

Risperidone was obtained from Sigma/Aldrich, St. Louis, Mo., dissolved in distilled water and pH was subsequently adjusted to 6.0 with lactic acid powder. Risperidone was then injected s.c. at a dose of 2.0 mg/kg according to the literature (Hertel et al., 1996; Hertel et al., 1998; Ichikawa et al., 1998; Ichikawa and Meltzer, 2000)). The doses of risperidone in the literature focussing on animals, shows that a low dose of 0.1/0.2 mg/kg or a high dose of 1.0/2.0 mg/kg s.c. are both valid selections. Although it is difficult to extrapolate doses from human to animal, it is believed that a 6-8 mg/day dose in humans may be equivalent to the 1.0/2.0 mg/kg dose in animals. Results of clinical trials have shown that 6-8 mg/day (orally) of risperidone were effective for most patients for treating psychotic symptoms without risking induction of EPS and this dose range was the recommended optimal daily dose (Marder and Meibach, 1994). Recently, though, on the basis of further clinical trials, even lower doses are now recommended, 4 mg/day (orally) (Williams, 2001).

At high dose risperidone, DA₂ receptor occupancy increases to 70%, while 5-HT₂ occupancy is maintained (90%) (Meltzer et al., 1992; Leysen et al., 1993; Schotte et al., 1993; Sumiyoshi et al., 1994; Svartengren and Calender, 1994; Sumiyoshi et al., 1995). Therefore, the rationale for selecting high dose risperidone in the present studies was based on the hypothesis that a greater DA₂ occupancy would have more potent DA₂ antagonist effects, postsynaptically and that fact, coupled with less DA released presynaptically, would be more efficacious in blocking cocaine-induced DA-ergic psychomotor stimulant effects.

Cocaine was obtained from Sigma Aldrich, St. Louis, Mo. and dissolved in distilled water. Cocaine was then injected i.p. at a dose of 10 mg/kg which is seen as a moderate dose and yet, one which will produce the psychostimulant effects of cocaine (Broderick et al., 1993; Van Campenhout et al., 1999; Filip and Cunningham, 2002).

### Surgical Procedures

Animals were purchased from Charles River Laboratories, Kingston, N.Y. and were housed in our animal care facilities for two weeks before surgery was performed. The Animal Care Facility operates under the auspices of the CUNY, City College Institutional Animal Care and Use Committee (IACUC) in compliance with National Institute of Health (NIH) guidelines. The weight range for the animals, at the time of the studies, was 350-475 g. Animals were group housed before surgery, individually housed after surgery and fed Purina Rat Chow and water ad libitum. A twelve hr dark-light cycle was maintained both in the housing of the animals and throughout the experimental studies. Each animal was anesthetized with pentobarbital Na, (50 mg/kg i.p. (dilute (6%) solution)) and stereotaxically implanted (Kopf Stereotaxic, Tujunga, Calif.) with a BRODERICK PROBE® lauric acid indicator microelectrode in ventrolateral (vl) NAcc (AP=+2.6, ML=+2.5, DV=−7.3) (Pellegrino et al., 1979). A Ag/AgCl reference electrode was placed in contact with dura, 7 mm anteriorally and contralaterally to the indicator microelectrode. A stainless steel auxiliary microelectrode, was placed in contact with dura.

Animals' body temperature was continuously monitored with a rectal probe and thermometer (Fisher Sci., Fadem, N.J.). Body temperature was maintained at 37.5° C.±0.5° C. with an aquamatic K module heating pad (Amer. Hosp. Supply, Edison, N.J.). Booster injections of pentobarbital Na were administered once after the first two hrs of surgery (0.10 cc) and once every subsequent hr (0.05 cc) to maintain an adequate level of anesthesia throughout surgery. The total time for surgery was three to four hrs. The indicator, reference, and auxiliary microelectrodes were held in place with dental acrylic (Jet Line, Lang Dental Inc., CA). Animals recovered in a bedded Plexiglas cage (dimensions: 12 inches (width), 12 inches (depth), 18 inches (height)) after surgery and before the experimental studies began, with food and water ad libitum. The animals were treated with physiological saline (0.5 cc) immediately and for one to two days after surgery as needed.

In vivo microvoltammetric studies on conscious Sprague-Dawley laboratory rats were begun nine to fifteen days after the aseptic surgical operations were performed. On each experimental day, animals were placed in a Plexiglas-copper faradaic chamber. The three-microelectrode assembly, enclosed within the animal's prosthetic acrylic cap, was connected to a CV37 detector by means of a mercury commutator (Br. Res. Instr., Princeton, N.J.), a flexible cable, and a mating connector (BJM Electronics, Staten Island, N.Y.). The CV37 detector was electrically connected to a Minigard surge suppresser (Jefferson Electric, Magnetek, N.Y.) which was then connected to an electrical ground in isolation. Stable electrochemical signals for DA and 5-HT were evident before either (i) risperidone (2 mg/kg s.c.), (ii) cocaine (10 mg/kg i.p.) or (iii) the combination of risperidone and cocaine (2 mg/kg s.c. and 10 mg/kg i.p. respectively) were administered. Each animal was used as its own control. In vivo microvoltammetric scans were recorded in sec and repeated every five min for a period of 2 hrs before each treatment and a period of two hrs after each treatment.

### Behavior

Behaviors were monitored with infrared photobeams which surrounded the faradaic chamber. Open-field behaviors of locomotion (ambulations) and stereotypy (fine movements of sniffing and grooming) were recorded in sec and repeated every five min for a period of 2 hrs before each treatment and a period of 2 hrs after each treatment. Open-field behaviors were monitored simultaneously with in vivo microvoltammetric recordings of monoamines.

The first hr pre-drug, allowed exploratory behavior. Exploratory behavior is defined as open-field behavior of ambulations (forward locomotion) and stereotypy (fine movements of sniffing and grooming), wherein animals respond to the stimuli of a novel environment with a high frequency of behavioral counts. The second hr pre-drug allowed the animal to become habituated before treatment. Habituation behavior is defined as a behavioral state in which neurochemistry and behavior exhibit reduced responses to novel stimuli; animals cease exploring or searching in their novel environment and maintain a steady-state response to novel stimuli. In the acute studies, each drug was administered thirty min into habituation. For the subacute studies, twenty four hrs later, the animals were again placed in the faradaic behavioral chamber and no further drug was administered. Each animal was monitored for possible recovery, withdrawal or after-effects. of each treatment WHILE open-field behaviors of locomotions and stereotypy were monitored with computerized infrared photocell beams which surround the faradaic chamber.

The faradaic chamber was made of plexiglas and covered with copper wire to refract possible electrical artifacts (dimensions: 24 inch (width) by 18 inch (depth) by 23.5 inch (height)). A 16 by 16 array of infrared photobeams, held in place by an aluminum frame, was situated ¾ inch above the Plexiglas floor of the chamber to detect locomotor and stereotypic movements. Photobeams were sampled by a Pentium computer to define the x-y position of the animal within a 1.5 inch resolution every 100 msec. When an x-y position was calculated, it was used to define a particular behavioral parameter. This system is a modified version of an Activity Pattern Monitor (APM) (San Diego Instruments, San Diego, Calif.). Behavioral data is presented in terms of Frequency of Events.

### In Vivo Microvoltammetry

In the present studies, in vivo microvoltammetry with a semidifferential circuit was used; a clear separation of the biogenic amine neurotransmitters, DA and 5-HT was achieved. Dopamine and 5-HT were detected within sec, in separate signals and in vivo. Oxidation peak potentials of +0.14±0.015V and +0.29±0.015V were characteristic for DA and 5-HT. Detailed methodology is published (Broderick, 1988; Broderick, 1989; Broderick, 1990; Broderick, 1991b; Broderick et al., 1993; Broderick, 1999; Broderick et al., 2000; Broderick, 2001; Broderick, 2002). The electrochemical signal for DA, was detected without interference at the same oxidation potential, from 3-4-dihydroxyphenylacetic acid (DOPAC), homovanillic acid (HVA) and ascorbic acid (AA). Indeed, clear and separated signals are achieved with the BRODERICK PROBE® lauric acid microelectrode, for AA, HVA and DOPAC; moreover, these clear and separated signals for AA, HVA and DOPAC are achieved with the BRODERICK PROBE® Stearic Acid Microelectrode as well. The electrochemical signal for 5-HT was detected without interference at the same oxidation potential, from the 5-HT metabolite, 5-hydroxyindoleacetic acid (5-HIAA) and uric acid (UA). Potentials were applied with a CV37 detector (BAS, West Lafayette, Ind.). Potentials were applied from −0.2V to +0.7V with respect to a Ag/AgCl (1M NaCl) electrode, at a scan rate of 10 mV/sec at time constants of 5 and 1 tau. One scan was completed in 60 sec. Non-faradaic charging current was eliminated in the first 25 sec. The neurotransmitters, DA and 5-HT, were detected in approximately 10-15 sec. and 10-12 sec., respectively, in a sequential manner during each recording. The coulombic efficiency for the detection of 5-HT was two to three fold greater than that for DA (Broderick, 1987).

Pre- and post-calibration curves were determined experimentally, in vitro, in a freshly prepared deoxygenated physiological saline-phosphate buffer solution, (0.01 M, pH=7.4 containing DA (99% purity, Sigma, St. Louis, Mo.) and 5-HT (99% purity, Aldrich, Milwaukee, Wis.), as well as metabolites of the monoamines (Broderick, 1989; Broderick et al., 2000). In vitro pre- and post-calibration of the indicator BRODERICK PROBE® were also studied in freshly prepared deoxygenated phosphotidylethanolamine (PEA)/bovine serum albumin (BSA) (Sigma Aldrich, St. Louis, Mo.) physiological saline-phosphate buffer which closely mimics brain constituents. These studies show that lipid constituents of brain amplify the detection sensitivity of the indicator microelectrodes, supporting previous data which show that lipids amplify electrochemical signals detected by BRODERICK PROBES®; the phenomenon is termed The Lipid Amplification Number (LAN) (Broderick, 1999; Broderick et al., 2000). Surface Enhanced Raman Spectroscopy (SERS) and Raman Resonance (RR) techniques have correlated our findings on signal amplification by lipids (Foucault et al., 2002). Detection limits for basal synaptic concentrations of DA and 5-HT in NAcc were 12 nM and 2 nM respectively. Placement of indicator microelectrodes in NAcc of each animal, was confirmed by the potassium ferrocyamide blue dot method, using a current of 50 mA for period of 40 sec. Virtually no damage to brain tissue occurred. Recording characteristics of microelectrodes were stable.

### Data Analysis

Neurochemical and behavioral data, derived from the last thirty minutes of the habituation period, provided the baseline data. Statistically significant differences between baseline and post-injection for (1) DA (2) 5-HT, (3) locomotor and (4) stereotypic behavior were determined by subjecting the data to One Way Analysis of Variance (ANOVA) (tested at p=0.05 as criteria), with subsequent application of the post hoc test, Tukey's Multiple Comparison Test. Where appropriate, data points in the time course were subjected to 95% Confidence Limits (C.L.).

Results

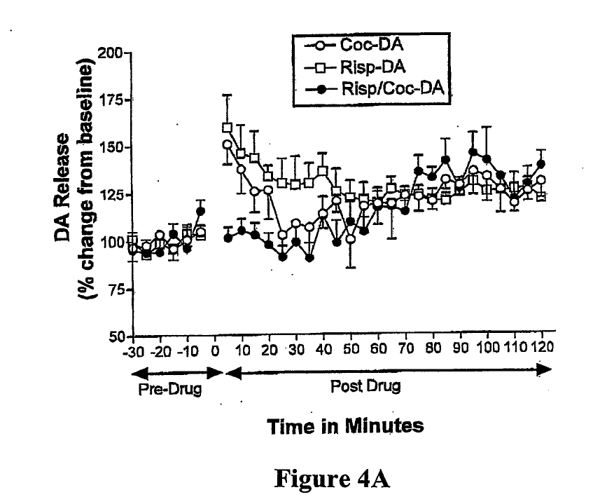

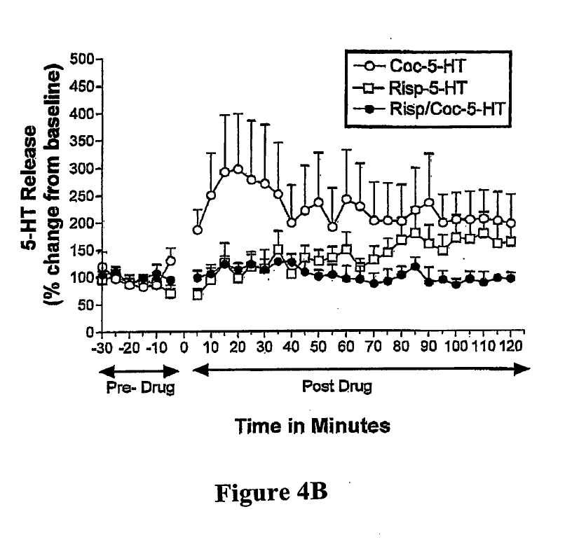

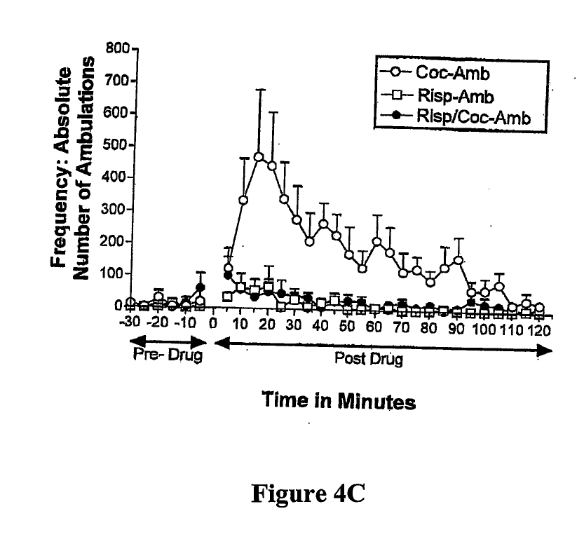

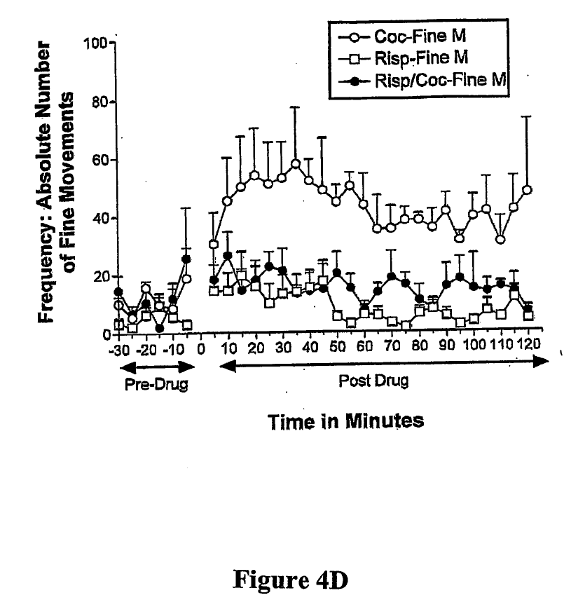

Day 1: Acute Studies: FIG. 4A: Effects of Risperidone, Cocaine or Risperidone/Cocaine Combination on DA Release in NAcc:

Risperidone: (open squares) Risperidone significantly increased DA release in NAcc over baseline (habituation) values (One Way ANOVA; p<0.0001; F=12.35; df=5.84). Post hoc analysis further showed that there were statistically significant differences between pre-risperidone (baseline) and post-risperidone (same animal control) (Tukey's Multiple Comparison Test: p<0.001, q=7.454)

Cocaine: (open circles) Cocaine significantly increased DA release over baseline (habituation) values (One Way ANOVA; p<0.0001; F=12.35; df=5.84). Post hoc analysis showed that significant differences between pre-cocaine (baseline) and post-cocaine (same animal control) occurred as well (Tukey's Multiple Comparison Test: p<0.01, q=5.649).

Risperidone/Cocaine: (closed circles) The risperidone/cocaine group exhibited a biphasic response on DA release in NAcc. Therefore, statistical analysis was extended in this group to perform analysis on an hr by hr basis. In the first hour, when DA release in NAcc was compared to baseline (habituation) values, a significant blockade of cocaine-induced DA release was observed since DA release was not different from baseline (Post hoc analysis (Tukey's Multiple Comparison Test: p>0.05, q=0.7423). Moreover, there was a significant difference between the results in the cocaine group versus the risperidone/cocaine group in the first hr which emphasizes the significant blockade of cocaine-induced DA release by risperidone (One Way ANOVA; p<0.0001; F=36.31; df=8.81); post hoc analysis further emphasizes this effect (Tukey's Multiple Comparison Test: p<0.01, q=6.085). In the second part of the biphasic response, DA release was increased above baseline to a statistically significant degree (One Way ANOVA; p<0.0001; F=36.31; df=8.81). Post hoc analysis showed significance also (Tukey's Multiple Comparison Test: p<0.001, q=9.650). Interestingly, then, in the second part of the biphasic response, since risperidone did not completely block cocaine-induced DA release, the risperidone/cocaine group did not significantly differ from the cocaine group as shown by post hoc analysis (Tukey's Multiple Comparison Test: p>0.05, q=1.964).

Day 1: Acute Studies: FIG. 4B: Effects of Risperidone, Cocaine and Risperidone/Cocaine Combination on 5-HT Release in NAcc:

Risperidone: (open squares) Risperidone significantly increased 5-HT release in NAcc over baseline (habituation) values (One Way ANOVA; p<0.0001; F=69.36; df=5.84). Post hoc analysis further showed that there were statistically significant differences between pre-risperidone (baseline) and post-risperidone (same animal control) (Tukey's Multiple Comparison Test: p<0.001, q=12.87)

Cocaine: (open circles) Cocaine significantly increased 5-HT release over baseline (habituation) values (One Way ANOVA; p<0.0001; F=69.36; df=5.84). Post hoc analysis showed that significant differences between pre-cocaine (baseline) and post-cocaine (same animal control) occurred as well (Turkey's Multiple Comparison Test: p<0.001, q=11.59).

Risperidone/Cocaine: (closed circles) The risperidone/cocaine group exhibited a monophasic and statistically insignificant response on 5-HT release in NAcc as shown by post hoc analysis (Tukey's Multiple Comparison Test: (p>0.05, q=0.1232). Thus, risperidone blocked cocaine-induced 5-HT release over the entire 2 hr period of study. A comparison of post hoc analysis of risperidone/cocaine effects versus cocaine effects on 5-HT release emphasizes the blockade of cocaine-induced 5-HT release (Tukey's Multiple Comparison Test: p<0.001, q=18.13).

Day 1: Acute Studies: FIG. 4C: Effects of Risperidone, Cocaine and Risperidone/Cocaine Combination on Locomotion (Ambulations)::

Risperidone: (open squares) Risperidone did not affect locomotor activity over baseline (habituation) values. Post hoc analysis showed that there were no statistically significant differences between pre-risperidone (baseline) and post-risperidone (same animal control) (Tukey's Multiple Comparison Test: p>0.05, q=0.4743)

Cocaine: (open circles) Cocaine significantly increased locomotor activity (ambulations) over baseline (habituation) values (One Way ANOVA; p<0.0001; F=21.06; df=5.84). Post hoc analysis showed that significant differences between pre-cocaine (baseline) and post-cocaine (same animal control) occurred as well (Tukey's Multiple Comparison Test: p<0.001, q=7.802).

Risperidone/Cocaine: (closed circles) The risperidone/cocaine group exhibited a statistically insignificant response on locomotor activity as shown by post hoc analysis (Tukey's Multiple Comparison Test: (p>0.05, q=0.4704). Thus, risperidone blocked cocaine-induced psychostimulant activity over the entire 2 hr period of study. A comparison of post hoc analysis of risperidone/cocaine effects versus cocaine effects on 5-HT release emphasizes the blockade of cocaine-induced locomotor activity (Tukey's Multiple Comparison Test: p<0.001, q=11.30).

Day 1: Acute Studies: FIG. 4D: Effects of Risperidone, Cocaine or Risperidone/Cocaine Combination on Stereotype (Fine Movements of Sniffing and Grooming):

Risperidone: (open squares) Risperidone did not affect stereotypy over baseline (habituation) values. Post hoc analysis showed that there were no statistically significant differences between pre-risperidone (baseline) and post-risperidone data (same animal control) (Tukey's Multiple Comparison Test: p>0.05, q=1.963).

Cocaine: (open circles) Cocaine significantly increased stereotypic behaviors of grooming and sniffing over baseline (habituation) values (One Way ANOVA; p<0.0001; F=101.7). Post hoc analysis showed that significant differences between pre-cocaine (baseline) and post-cocaine (same animal control) occurred as well (Tukey's Multiple Comparison Test: p<0.001, q=16.29).

Risperidone/Cocaine: (closed circles) The risperidone/cocaine group exhibited a statistically insignificant response on stereotypy as shown by post hoc analysis (Tukey's Multiple Comparison Test: (p>0.05, q=1.813). Thus, risperidone blocked cocaine-induced psychostimulant stereotypic activity over the entire 2 hr period of study. A comparison of post hoc analysis of risperidone/cocaine effects versus cocaine effects on stereotypy emphasizes the blockade of cocaine-induced stereotypy activity (Tukey's Multiple Comparison Test: p<0.001, q=22.48).

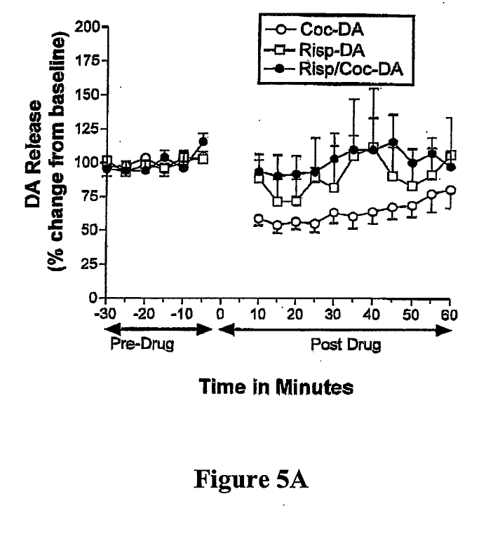

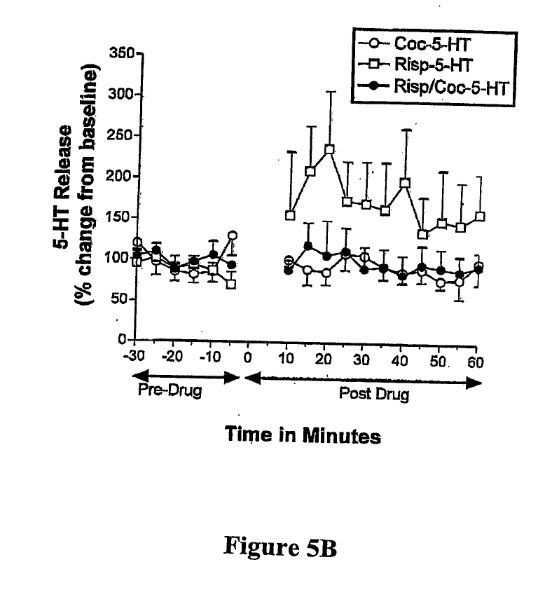

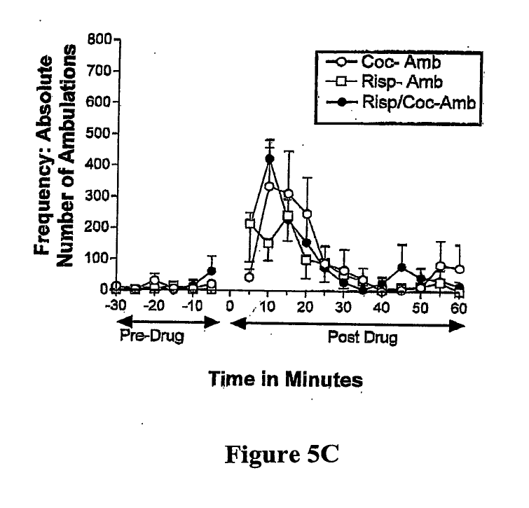

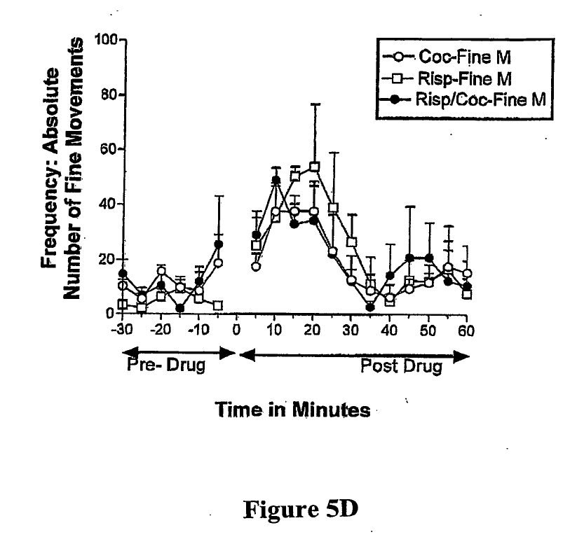

Day 2: Subacute Studies: FIG. 5A: Effects of Risperidone, Cocaine or Risperidone/Cocaine Combination on DA Release in NAcc:

Risperidone: (open squares) During the subchronic studies, when no further risperidone was administered, DA release in NAcc returned to baseline (habituation) values (from a significant increase); thus, there was no significant difference between baseline (Day1) and (Day2) values (same animal control), as shown by post hoc analysis (Tukey's Multiple Comparison Test: p>0.05, q=2.797).

Cocaine: (open circles) During the subchronic studies, when no further cocaine was administered, DA release in NAcc significantly decreased from baseline (habituation) values (from a significant increase) (One Way ANOVA; p<0.0001; F=24.65; df=5.45). Post hoc analysis showed that significant differences occurred between baseline (Day1) and (Day2) values (same animal control) (Tukey's Multiple Comparison Test: p<0.001, q=10.85).

Risperidone/Cocaine: (closed circles) During the subchronic studies, when no further risperidone/cocaine combination was administered, DA release in NAcc returned to baseline (habituation) values (from a biphasic response); there was no significant difference between baseline (Day1) and (Day2) values (same animal control), as shown by post hoc analysis (Tukey's Multiple Comparison Test: p>0.05, q=0.3592). There were significant differences in DA concentrations in NAcc between the risperidone/cocaine group on Day2 as compared with the cocaine group on Day2 (One Way ANOVA; p<0.0001; F=24.65; df=5.45). Post hoc analysis further emphasize the ability of risperidone to block cocaine effects, even subchronically during timing that corresponds to cocaine withdrawal (Tukey's Multiple Comparison Test: p<0.001, q=13.34).

Day 2: Subacute Studies: FIG. 5B: Effects of Risperidone, Cocaine and Risperidone/Cocaine Combination on 5-HT Release in NAcc:

Risperidone: (open squares) During the subchronic studies, when no further risperidone was administered, 5-HT release in NAcc significantly increased above Day1 baseline values (One Way ANOVA; p<0.0001; F=32.26; df=5.45). Post hoc analysis showed that significant differences occurred between baseline (Day1) and (Day 2) values (same animal control) (Tukey's Multiple Comparison Test: p<0.001, q=12.91). Compared to drug effect of risperidone on Day 1, the significant increase in 5-HT release was maintained and increased further by an approximate average of 25%.

Cocaine: (open circles) During the subchronic studies, when no further cocaine was administered, 5-HT release in NAcc was decreased below Day1 baseline values at specific time points during the time course of the 1 hr study, i.e., at the 15, 20,50,55 min time points, although the post hoc analysis did not show statistical significance (Tukey's Multiple Comparison Test: p>0.05, q=11.118). Compared to drug effect on Day1, 5-HT release was decreased dramatically by about 75%.

Risperidone/Cocaine: (closed circles) During the subchronic studies, when no further risperidone/cocaine treatment was administered, 5-HT release in NAcc exhibited exactly the same profile as was seen on Day1, i.e., on Day1 and on Day2, 5-HT release did not exhibit a statistically significant change from baseline. Thus, post hoc analysis shows no difference between 5-HT release between Day1 and Day2 data (Tukey's Multiple Comparison Test: (p>0.05, q=0.4579). No significant differences occurred between risperidone/cocaine and cocaine groups (Tukey's Multiple Comparison Test: (p>0.05, q=1.20). A significant difference in 5-HT release between risperidone/cocaine and risperidone groups occurred (Tukey's Multiple Comparison Test: (p<0.001, q=11.31)

Day 2: Subacute Studies: FIG. 5C: Effects of Risperidone. Cocaine and Risperidone/Cocaine Combination on Locomotion (Ambulations):: One-Way ANOVA for all Groups Tested, Showed a Significance of D<0.05; F=2.377: df=5.48.

Risperidone: (open squares) During the subchronic studies, when no further risperidone was administered, and Day 1 baseline (habituation) values were compared with Day 2 values, risperidone did not affect locomotion to a statistically significant degree. Post hoc analysis showed that there were no statistically significant differences between groups (same animal control) (Tukey's Multiple Comparison Test: p>0.05, q=2.271). Nonetheless, compared with drug treatment, Day1, locomotor activity was increased from a frequency of 50 to about 200 counts.

Cocaine: (open circles) During the subchronic studies, when no further cocaine was administered, and Day1 baseline (habituation) values were compared with Day2 values, post hoc analysis showed that there were no statistically significant differences between groups (same animal control) (Tukey's Multiple Comparison Test: p>0.05, q=2.2949). Interestingly, compared with drug treatment, Day1, locomotor activity was decreased by a frequency of about 200 counts.

Risperidone/Cocaine: (closed circles) During the subchronic studies, when no further risperidone/cocaine were administered, and Day1 baseline (habituation) values were compared with Day2 values, locomotion was not affected to a statistically significant degree. Post hoc analysis showed that there were no statistically significant differences between groups (same animal control) (Tukey's Multiple Comparison Test: p>0.05, q=2.869). Again, interestingly, compared with drug treatment, Day1, locomotor activity was increased by a frequency of about 200 counts. No significant difference in locomotion occurred between risperidone/cocaine and cocaine groups occurred (Tukey's Multiple Comparison Test: p>0.05, q=0.05135).

Day 2: Subacute Studies: FIG. 5D: Effects of Risperidone, Cocaine or Risperidone/Cocaine Combination on Stereotypy (Fine Movements of Sniffing and Grooming).: One-Way ANOVA for all Groups Tested, Showed a Significance of P<0.01; F=3.09; df=5.48.

Risperidone: (open squares) During the subchronic studies, when no further risperidone was administered, and Day1 baseline (habituation) values were compared with Day2 values, risperidone increased stereotypy to a statistically significant degree (One-Way ANOVA: p<0.01; F=3.09; df=5.48). Post hoc analysis further showed that there were statistically significant differences between groups (same animal control) (Tukey's Multiple Comparison Test: p<0.05, q=4.622). Compared with drug treatment, Day 1, stereotypy was increased from a maximum frequency of 12 to a maximum frequency of about 50 counts.

Cocaine: (open circles) During the subchronic studies, when no further cocaine was administered, and Day 1 baseline (habituation) values were compared with Day 2 values, post hoc analysis showed that there were no statistically significant differences between groups (same animal control) (Tukey's Multiple Comparison Test: p>0.05, q=1.947). Interestingly, compared with drug treatment, Day1, stereotypy was decreased by a frequency of about 20 counts from 60-40 counts.

Risperidone/Cocaine: (closed circles) During the subchronic studies, when no further risperidone/cocaine were administered, and Day1 baseline (habituation) values were compared with Day2 values, stereotypy was not affected to a statistically significant degree. Post hoc analysis showed that there were no statistically significant differences between groups (same animal control) (Tukey's Multiple Comparison Test: p>0.05, q=2.327). Again, interestingly, compared with drug treatment, Day1 stereotypy was increased from a frequency of 12 to about 30 counts. A significant difference between risperidone/cocaine and cocaine groups did not occur (Tukey's Multiple Comparison Test: p>0.05, q=0.6102).

Discussion

The Anatomy of Schizophrenia: Although the precise biological basis of schizophrenia remains to be fully elucidated, several repetitive observations have reliably evolved into a theory of psychosis called, “The Mesolimbic DA Hypothesis of Positive Psychotic Symptoms”. It is thought that excessive activity in this neuronal pathway, which projects from the somatodendritic DA cell bodies in the VTA to axon terminals in the limbic area of the forebrain, NAcc, mediates the positive symptoms of psychosis. Simply stated, the psychostimulant, cocaine, is thought to induce positive symptoms of psychosis by increasing DA activity to an excessive degree in mesolimbic terminals, NAcc (Stahl, 2000). It is important to note that 5-HT modulates DA in the mesolimbic circuit and there are significant concentrations of 5-HT_{1B}, 5-HT_{2A}, 5-HT_{2C} and 5-HT₃ receptors present in NAcc (Leysen et al., 1996).

On the other hand, The DA Mesocortical Pathway originates in DA somatodendrites, VTA also, but this projects to prefrontal cortex (PFC) nerve terminals. This mesocortical substrate is thought to play a major role in the negative symptoms of psychosis similar to defects seen after frontal lobectomy. Weinberger et al. (1992) showed that a reduction in PFC DA-ergic activity leads to disinhibition and overactivity of DA-ergic function in mesolimbic circuitry (Weinberger et al., 1992). Moreover, 5-HT modulates DA in PFC as well, even to a greater degree than occurs in NAcc (Meltzer, 1999) and significant concentrations of 5-HT_{1A}, 5-HT_{1B}, 5-HT_{2A}, 5-HT₃ and 5-HT₇ receptors are present in PFC (Leysen et al., 1996). This research presents a focus on positive symptoms of psychosis since NAcc is the substrate of interest.

Risperidone:

Acute Studies: Results were as expected. Consistent with risperidone studies from other laboratories wherein microdialysis was used, risperidone significantly increased DA concentrations in NAcc. (Volonte et al., 1997; Kuroki et al., 1999). The latter study reported the mechanism of action of increased efflux of DA in NAcc to derive from a combination of 5-HT_{2A}/DA₂ effects plus a weak DAD_{2/3} affinity relative to 5-HT_{2A} but the mechanism was not thought to be directly due to a 5-HT_{2A} mediation of DA release (Kuroki et al., 1999). Interestingly, a DAD_{2/3} affinity may be explanatory. (+)-AJ 76 [cis-(+)-1S,2R-5 methoxy-1 methyl-2-(n-propylamino)-tetralin HCL] is a DA autoreceptor antagonist with a slightly higher affinity for the DA₃ rather than the DA₂ receptor (Sokoloff et al., 1990) and this laboratory and others have found that AJ 76, unlike other typical antipsychotic agents, has weak stimulant properties as opposed to sedative properties (Waters et al, 1993; Broderick and Piercey, 1998b).

Also consistent with the present results, is another report which showed, using microdialysis, that risperidone increased release of DA in NAcc (Ichikawa and Meltzer, 2000). In this last report, the data suggested that a 5-HT_{1A} receptor mediation was not involved in the mechanism of action of increased DA release after high dose risperidone, as these increases were not antagonized by the 5-HT_{1A} receptor agonist, (8-OH-DPAT) [R(+)-8-hydroxy-2-(di-n-propylamino)-tetralin]. Although risperidone does not bind with high affinity to 5-HT_{1A} receptors, interestingly, low dose risperidone did show a 5-HT_{1A} mediation in the mechanism of action of increased DA release by risperidone (Ichikawa and Meltzer, 2000). Furthermore, inverse agonist activity at the 5-HT_{2C} receptor, may play a role in risperidone induced increases DA release in NAcc as risperidone completely prevented the inhibitory action of RO 60-0175, a 5-HT_{2C} receptor agonist, on DA efflux in NAcc (Di Matteo et al., 2002). Therefore, the increase in DA release in NAcc after risperidone as reported by this laboratory, may come in part from somatodendritic presynaptic antagonism of DA and/or 5-HT autoreceptors, and in part from postsynaptic 5-HT_{2C} modulation of DA. Taken together with Ichikawa and Meltzer (2000), a 5-HT_{1A} mediation may not be likely since our risperidone dose could be considered a high dose. A DA₃ autoreceptor antagonism should not be ruled out as the mesolimbic neuronal circuitry has a high concentration of DA₃ receptors (Sokoloff et al., 1990).

Results were as expected and consistent with previous studies of risperidone on 5-HT release in NAcc. Thus, the present data are in agreement with others (Ichikawa et al., 1998) who reported, using microdialysis, an increased 5-HT efflux from NAcc albeit statistically insignificant. The latter report discusses the mechanism of increased 5-HT efflux after risperidone, as related to 5-HT reuptake inhibition and not directly due to a 5-HT_{2A} receptor mediation of 5-HT-induced increases within the DA terminals in NAcc, although the authors state that an interaction with DA₂ receptor mediation is possible (Ichikawa et al., 1998). Too, in another report, risperidone was studied, not for its effects on 5-HT, the neurotransmitter, in NAcc but for risperidone's effects on the metabolite of 5-HT, i.e., 5-hydroxyindoleacetic acid (5-HIAA). Both low and high dose risperidone (0.2 mg/kg s.c. and 2.0 mg/kg s.c.) were studied with similar results. The results were interesting in that the increase in 5-HIAA concentrations, up to 20% above baseline, were time-dependent (Hertel et al., 1996). The results of the present studies showed that the increase in 5-HT release in NAcc after risperidone, also increased during the time course, up to about 60%. These data are interesting in that there may be an implication of decreased 5-HT turnover after risperidone.

The present results are also in agreement with another report which showed that risperidone at a dose range of 25-400 ug/kg i.v., increased 5-HT efflux locally in 5-HT somatodendrites, consequent to decreased activation of 5-HT_{1A} autoreceptor mediated cell firing in 5-HT somatodendrites (Dorsal Raphe (DR)) (Hertel et al., 1997b). This latter report suggests that the availability of 5-HT in somatodendrites provides a plausible mechanism for increased 5-HT release after risperidone since the 5-HT depletor, parachlorophenylalanine (PCPA) also depresses 5-HT_{1A} firing in DR somatodendrites (Hertel et al., 1997b; Hertel et al., 1998). In addition, other nerve terminals in the DA-ergic mesocorticolimbic neuronal circuitry, PFC, exhibited increased 5-HT concentrations after risperidone administrations (Hertel et al., 1997a; Ichikawa et al., 1998; Hertel et al., 1999).

Finally, since direct receptor acting 5-HT_{2A/C} autoreceptor agonists, such as DOI [(+/−)-2,5-dimethoxy-4-iodoamphetamine hydrochloride], decrease 5-HT release in PFC (Wright et al., 1990) and direct receptor acting 5-HT_{2AC} receptor antagonists increase 5-HT release in NAcc (Devaud et al., 1992), another plausible mechanism of a 5-HT-ergic increase in NAcc is 5-HT_{2A/C} mediation of 5-HT release by direct acting autoreceptor antagonist action in DA mesolimbic neuronal circuitry.

Risperidone Behavior: It is believed that this is the first report on the behavioral effects of risperidone preclinically; thus, we are without comparison in this area. However, we found that the open-field behaviors of locomotion and stereotypy were affected insignificantly by risperidone although there was an increase in frequency of counts over baseline in the first hr.

Subacute Studies: During the 24 hr follow-up studies, when no further drug was administered, risperidone produced a significant increase in 5-HT release in NAcc. This is an exciting finding as increased 5-HT release in the DA-ergic mesolimbic pathway could be beneficial for treatment of schizoaffective disorders, which includes depressive components. The increase in 5-HT, which appears to be specific to 5-HT, may be explained by 5-HT_{1A} autoreceptor inhibition presynaptically since risperidone is still present due to the prolonged half-life of risperidone's metabolite, 9-hydroxyrisperidone (Mannens et al., 1993). Dopamine and locomotion returned to baseline but stereotypy, an A₉ behavior (Kelly et al., 1975), increased above baseline.

Cocaine:

Acute Studies: Cocaine exhibits a high affinity for DA, 5-HT and NE transporters and via these transporters, reuptake of monoamines into presynaptic nerve terminals is inhibited (Koe, 1976); interestingly, certain subjective reward and jittery effects from cocaine have recently been associated with these monoamine transporters (Hall et al., 2002). Too, the mechanism of action of cocaine has been shown to be dependent on stimulated release mechanisms (Ng et al., 1991) and on basal release mechanism using the DA impulse flow inhibitor, γBL (Broderick, 1991b). Nonetheless, although cocaine is a DA reuptake inhibitor and not a direct receptor acting agonist, enhancement of DA neurotransmission may also be provided adjunctly through indirect activation of DA receptors, i.e., D₁ and D₂ (Spealman et al., 1992; Wise, 1995).

Therefore, as expected and consistent with previous data from this laboratory and others, cocaine produced significant increases in synaptic DA concentrations in NAcc (de Wit and Wise, 1977; Church et al., 1987; Ritz et al., 1987; Bradberry and Roth, 1989; Hurd and Ungerstedt, 1989; Kalivas and Duffy, 1990; Broderick, 1991a,b; Broderick, 1992a,b; Broderick et al., 1993, Broderick and Piercey, 1998b). It is well accepted that the DA mesolimbic neuronal pathway, from VTA to NAcc is critical for the action of cocaine as intra-NAcc administration of cocaine into this area mimics among other cocaine behaviors, reinforcing effects (McKinzie et al., 1999), discriminative stimuli (Callahan et al., 1994) and consistent with the present data, the hyperlocomotive effects of systemic cocaine (Delfs et al., 1990). Attenuation of psychostimulant behaviors is generally thought to be via DA₂ receptors postsynaptically.

Consistent with previous data from this laboratory and others, cocaine produced its expected increases in synaptic concentrations of 5-HT in NAcc (Broderick, 1992a; Broderick, 1992b; Broderick et al. 1993; Bradberry et al. 1993; Essman et al., 1994; Parsons et al., 1996; Tenaud et al., 1996; Broderick et al., 1997; Reith et al., 1997; Broderick, 2001; Andrews and Lucki, 2001), in concert with locomotor and stereotypic behavior (Broderick, 2001). The data are in agreement with others in that 5-HT-ergic agonist manipulations, such as 8-OH-DPAT, have been shown to upmodulate cocaine-induced psychostimulant behavior (De La Garza and Cunningham, 2000). In other types of 5-HT manipulations such as the animal model of 5-HT deficiency, i.e., the Fawn-Hooded rat, cocaine-induced increases in 5-HT release were attenuated (Hope et al., 1995). Finally, blockade of cocaine-induced hyperactivity has been attributed to postsynaptic antagonism of several 5-HT receptor subtypes, i.e., a 5-HT₄, 5-HT_{2A} and 5-HT_{2C} receptor mediation in NAcc (McMahon and Cunningham, 1999; McMahon and Cunningham, 2001; Filip and Cunningham, 2002).

Consensus on the mechanism of action of cocaine on DA and 5-HT release in NAcc with concomitant psychostimulant behavior implicates a postsynaptic 5-HT-ergic modulation of DA in the DA-ergic mesolimbic circuit. Cocaine sensitization mechanisms show that 5-HT_{2A} receptors mediate DA release in NAcc (Yan et al., 2000). But further teasing apart of the 5-HT_{2A} versus 5-HT_{2C} receptor activation came from studies with the selective 5-HT_{2A} antagonist, 463499B, versus the selective 5-HT_{2C} antagonist, RS 102221. The studies showed that the 5-HT_{2A} antagonist, (Lucas and Spampinato, 2000) did not block (DOI) increases in DA release but the 5-HT_{2C} antagonist did so (McMahon et al., 2001). Thus, perhaps, increased DA release by cocaine as well as its closely correlated psychostimulant behavior, may be a postsynaptically mediated phenomenon by 5-HT_{2C} receptors with additional DA release derived presynaptically from DA somatodendritic 5-HT_{2A} autoreceptors activation in VTA with consequent feedback compensation (Filip and Cunningham, 2002).

Subacute Studies: During the 24 hr follow-up studies, when no further drug was administered, the cocaine group showed an overall significant decrease in DA release in NAcc and significant decreases in 5-HT release during specific points in the time course data. These data are in agreement with others (Parsons et al. 1995; Parsons et al. 1996; Broderick et al., 1997). Since the behavioral activity increased at the same time that accumbens DA and 5-HT release were decreased, these subacute withdrawal data show that dissociative function between behavior mediated postsynaptically versus what is likely a monoamine deficiency presynaptically perhaps via autoreceptors. Thus, the data may help elucidate pharmacotherapeutic strategies which may provide beneficial responses on the neurochemical level without affecting already beneficial responses on the behavioral level, such as those seen here.

The present data are consistent with patient reports of withdrawal symptoms including craving, which from the clinical perspective, has been associated with reduction in DA neurotransmission (Blum et al., 1989). Craving is such an important issue to address because craving for cocaine can reemerge, even months or years after the last episode of cocaine use; in fact, cocaine craving can occur in association with affective (mood) states either positive or negative symptomotologies, geographic locations, specific persons or events, intoxication with other substances or in the presence of various objects directly or indirectly connected with cocaine use (Dackis and Gold, 1985; Gawin and Kleber, 1986b; Blum et al., 1989).

Therefore, the clinical data suggest that cocaine-induced neuroadaptation occurs; cocaine-induced neuroadaptation has been reported preclinically (Koob and Nestler, 1997, Broderick, 2001). It is of particular importance to show neuroadaptation by cocaine because, unlike amphetamine, neurodegeneration and neurotoxicity is difficult to prove empirically (Ryan et al., 1988; Seiden and Kleven, 1988). Since 5-HT release is also decreased below baseline, the data also suggest a biochemical basis for clinical depression observed in cocaine addicts during withdrawal (Price et al., 2001).

It is not likely that active metabolites that play a role in decreased DA and 5-HT release in NAcc during withdrawal from the single injection of cocaine. Although there are active cocaine metabolites e.g., norcocaine and benzoylecgonine in rat brain (Misra et al., 1974a; Nayak et al., 1976) the pharmacokinetic half-lives of these metabolites are short-lived. Indeed, the pharmacokinetic half-life of cocaine is short-lived (Nayak et al., 1976). The elimination half-lives of cocaine and norcocaine after bolus injections (e.g., 14.7 umol/kg) are similar (28-33 min) and for benzoylecgonine was 40-44 min (Mets et al., 1999). In addition, even benzoyl methyester exhibited a half-life of 60-71 min. (Mets et al., 1999). After i.v. and p.o. cocaine, (20-40 mg/kg), norcocaine was not detected at all in brain after i.v. cocaine but serum levels were as high as those of oral cocaine (Sun and Lau, 2001). In a landmark paper, (Nayak et al., 1976) i.v. cocaine was reported to have a half-life of 0.5 hr, and for s.c. that of 4 hrs, whereas that of i.p. cocaine can be extrapolated to approximately 2 hrs. Since the withdrawal subchronic studies took place approximately 24 hr after drug, it is reasonable to assume that virtually neither cocaine nor its principal active metabolites could play a role in the decreased effect seen in the monoamines. However, one may speculate that toxic metabolites, norcocaine nitroxide from norcocaine is a possibility (Kloss et al., 1984); since behavior recovered though, this does not seem likely. Finally, neuroadaptation as a prelude to neurodegeneration might lend an explanatory note (Koob and Nestler, 1997; Broderick, 2001).

Risperidone/Cocaine:

Acute Studies: The present studies are the first to report the effects of the atypical antipsychotic medication, risperidone, on accumbens DA and 5-HT release and simultaneous psychostimulant behavior induced by cocaine. Therefore, we have no comparative data in this area. Nonetheless, our results showed that risperidone significantly antagonized cocaine-induced enhancement in DA release albeit only during the first hr. Cocaine-induced enhancement of 5-HT release was blocked during both hrs of study, while at the same time, risperidone antagonized the psychomotor stimulant behaviors of locomotion and stereotypy produced by cocaine.

Curious are the data gleaned during the second hr of the combination risperidone/cocaine study. Dopamine release induced by cocaine, was not completely blocked in the second hr of study, but blockade of 5-HT release and psychomotor stimulant behaviors of locomotion and stereotypy was maintained during the 2 hr period of study. At first glance, perhaps the pharmacokinetics of risperidone and/or cocaine may provide a rational explanation? In humans, the half-life of risperidone is 3 hrs for fast metabolizers and 20 hrs for slow metabolizers. Although we do not know the rate of metabolism for risperidone in the laboratory rat, we do know that the 9-hydroxyrisperidone metabolite is active for 22 hrs and this metabolite is active and independent of the rate of metabolism (Janssen et al., 1988; Keegan et al., 1994). Conversely, the half-life for cocaine is about 2 hrs after i.p. injection and metabolites' half-life is less than 1 hr (Nayak et al. 1976; Mets et al., 1999; Sun and Lau, 2001). Therefore, pharmacokinetic data from both risperidone and cocaine in acute studies, show that both drugs are in the induction phase and these data do not lend an explanatory note to this phenomenon of incomplete blockade of cocaine-induced DA release by risperidone.

Apparently specific to the DA response, one may postulate that risperidone ceased to block cocaine-enhanced release by triggering presynaptic autoreceptor action at DA or 5-HT somatodendrites. Whether or not, this is due to changing 5-HT-ergic modulation of DA by high dose risperidone due to differential 5-HT_{2A}/DA₂ receptor occupancy at this dose or in this neuroanatomic region, it is premature to say. Since a high dose risperidone maintains a nearly maximal 5-HT_{2A} receptor occupancy (90%) but increases occupancy of DA₂ receptors to 70% (Leysen et al., 1993; Schotte et al., 1993; Meltzer et al., 1992; Sumiyoshi et al., 1994, Svartengren and Celander, 1994; Sumiyoshi et al., 1995), what was expected was a more potent DA₂ antagonism of cocaine-induced DA release. Thus, the exact reason for this second hr DA phenomenon, cannot be conclusively derived from the present experiments and further study will provide answers as to whether or not this phenomenon is exclusive to the typicality of the high dose nature of risperidone.

Moreover, the complexity of the receptor profile of risperidone should be factored into the equation. This cannot be ruled out because effects at the 5-HT_{1A} receptor and inverse agonist activity at 5-HT_{2C} receptors have been reported for risperidone (Ichikawa and Meltzer, 2000; DiMatteo et al. 2002, respectively), nor interaction with α₁ and α₂ receptors as well as histamine H₁ receptors be ruled out because of risperidone's high affinity at these receptors (Janssen et al., 1988; Leysen et al., 1988; Leysen et al., 1992).

Nonetheless and importantly, though, the present data may well help elucidate risperidone as a pharmacotherapeutic strategy for cocaine because this moderately enhanced DA release presynaptically may help allay craving for cocaine while the postsynaptic blockade response may reduce euphoria from cocaine (Broderick and Piercey, 1998b).

Subacute Studies: During the 24 hr studies, when no further drug was administered, both DA and 5-HT release in NAcc returned to baseline while behavioral parameters were increased insignificantly above baseline. The deficiencies in monoamine release produced by cocaine appear to have been alleviated by the atypical antipsychotic, risperidone. Withdrawal deficiencies such as 5-HT were down-modulated, an effect which may be helpful in depression consequent to cocaine withdrawal. The subacute response to the combination, risperidone and cocaine treatment may again be reasoned by the longer half-life of the risperidone metabolite, 9-hydroxyrisperidone (Mannens et al. 1993) and probably not the shorter half-life of cocaine (Nayak et al., 1976). Alternatively, we may invoke the response of DAT and SERT to risperidone in NAcc; these transporter proteins resist inactivation after treatment with risperidone and monoamines remain longer in the synapses for activation at pre and post-synaptic effects (Tarazi et al., 2000).

### CONCLUSIONS

Results from in-depth in vivo microvoltammetric and behavioral studies on three acute and three subacute studies, involving risperidone, cocaine and risperidone/cocaine combination have provided the following pertinent conclusions. (a) Risperidone's enhanced 5-HT release subacutely may prove valuable in the treatment of the depressive aspects of schizoaffective disorders. (b) Cocaine produced withdrawal symptoms most dramatically in DA and 5-HT release during subacute studies, likely due to neuroadaptive mechanisms. (c) Risperidone's blockade of cocaine-enhanced neurochemistry and behavior during acute studies, and amelioration of cocaine withdrawal effects, subacutely, suggests that risperidone may present a viable pharmacotherapy for cocaine addiction, psychosis and withdrawal.

### Abbreviations

Some terms used in the instant disclosure have been abbreviated herein as follows: activity pattern monitor (APM); ascorbic acid (AA); bovine serum albumin (BSA); dihydroxyphenylacetic acid (DOPAC); [(+/−)-2,5-dimethoxy-4-iodoamphetamine hydrochloride] (DOI); dopamine (DA); Dorsal Raphe (DR); Extrapyramidal Symptoms (EPS); gamma-butyrolactone (γBL); homovanillic acid (HVA); [R(+)-8-hydroxy-2-(di-n-propylamino)-tetralin] (R(+)-8-OH-DPAT); 5-hydroxyindoleacetic acid (5-HIAA); Institutional Animal Care and Use Committee (IACUC); mesolimbic pathway; mesocorticolimbic neuronal pathway (A₁₀); nigrostriatal neuronal pathway (A₉); norepinephrine (NE); Nucleus Accumbens (NAcc); phosphotidylethanolamine (PEA); picoamperes (pA); Prefrontal Cortex (PFC); Raman Resonance (RR); serotonin (5-HT); silver/silver chloride (Ag/Ag/Cl); Subacute studies (24 hr follow-up studies); Surface Enhanced Raman Spectroscopy (SERS); tyrosine hydroxylase (TH); uric acid (UA); Ventral Tegmental Area (VTA); ventrolateral Nucleus Accumbens (vlNAcc). affinity constant (Ki); [cis-(+)-1S,2R-5 methoxy-1 methyl-2-(n-propylamino)-tetralin HCL] (AJ76); American Psychiatric Association (APA); dopamine_{2,3} receptors (DAD_{2/3}); dopamine transporter protein (DAT); Effective Dose [50%] (ED₅₀); Extrapyramidal Symptoms (EPS); parachlorophenylalanine (PCPA); serotonin transporter protein (SERT); Single Photon Emission Computerized Tomography (SPECT); Striatum (Str); subacute studies (24 hr follow-up studies).

The following citations provide helpful background information and are incorporated herein in their entirety by reference.

### REFERENCES

Allen, S. M., Freeman, J. N., Davis, W. M., 1998. Evaluation of risperidone in the neonatal 6-hydroxydopamine model of Lesch-Nyhan Syndrome. Pharmacol. Biochem. Behav. 59 (2) 327-330.
American Psychiatric Association, 2000. Diagnostic and Statistical Manual of Mental Disorders, DSM IV-TR. Amer. Psychiat. Assn., Wash. D.C.
Andrews, C. M., Lucki, I., 2001. Effects of cocaine on extracellular dopamine and serotonin levels in the nucleus accumbens. Psychopharmacology, (Berl.) 155 (3), 221-229
Beart, P. M., McDonald, D., 1982. 5-Hydroxytryptamine and 5-hydroxytryptaminergic-dopaminergic interactions in the ventral tegmental area of rat brain. J. Pharm. Pharmacol. 34, 591-593.
Bersani, G., Grispini, A., Marini, S., Pasini, A., Valducci, M., Clani, N., 1986. Neuroleptic-induced EPS: clinical perspectives with ritanserin (R 55667), a new selective 5-HT2 receptor blocking agent. Curr. Ther. Res. 40, 492-499.
Blum, K., Trachtenberg, M. C., Kowzlowski, G. P., 1989. Cocaine therapy: the ‘reward cascade’ link. Prof. Counselor Jan./Feb., 327-335.
Bradberry, C. W., Nobiletti, J. B., Elsworth, J. D., Murphy, B., Jatlow, P., Roth, R. H., 1993. Cocaine and cocaethylene: microdialysis comparison of brain drug levels and effects on dopamine and serotonin. J. Neurochem. 60, 1429-1435.
Bradberry, C. W., Roth, R. H., 1989. Cocaine increases extracellular dopamine in rat nucleus accumbens and ventral tegmental area as shown by in vivo microdialysis. Neurosci. Lett. 103, 97-102.
Brady, K. T., Lydiard, R. B., Malcolm, R., Ballenger, J. C., 1991. Cocaine-induced psychosis. J. Clin. Psychiat. 52, 509-512.
Broderick, P. A. Piercey, M. F., 1998b. Neurochemical and behavioral evidence supporting (+)-AJ 76 as a potential pharmacotherapy for cocaine abuse. J. Neural. Transm. 105, 1307-1324.
Broderick, P. A., 1987. Striatal neurochemistry of dynorphin-(1-13): in vivo electrochemical semidifferential analyses. Neuropeptides 10, 369-386.
Broderick, P. A., 1988. Distinguishing in vitro electrochemical signatures for norepinephrine and dopamine. Neurosci. Lett. 95, 275-280.
Broderick, P. A., 1989. Characterizing stearate probes in vitro for the electrochemical detection of dopamine and serotonin. Brain Res. 495, 115-121.
Broderick, P. A., 1990. State-of-the-art microelectrodes for in vivo voltammetry. Electroanalysis 2, 241-251.
Broderick, P. A., 1991a. Cocaine: on-line analysis of an accumbens amine neuronal basis for psychomotor behavior. Pharmacol. Biochem. Behav. 40, 959-968.
Broderick, P. A., 1991b. In vivo voltammetric studies on release mechanisms for cocaine with γ-butyrolactone. Pharmacol. Biochem. Behav. 40, 969-975.
Broderick, P. A., 1992a. Cocaine's colocalized effects on synaptic serotonin and dopamine in ventral tegmentum in a reinforcement paradigm. Pharmacol. Biochem. Behav. 42, 889-898.
Broderick, P. A., 1992b. Distinguishing effects of cocaine (IV) and (SC) on mesoaccumbens dopamine and serotonin release with chloral hydrate anesthesia. Pharmacol. Biochem. Behav. 43, 929-937.
Broderick, P. A., 1999. Microelectrodes and their use in an electrochemical arrangement with telemetric application. U.S. Pat. No. 5,938,903. Representative of approximately 20 patents and trademarks.
Broderick, P. A., 2001. Microsensors detect cocaine neuroadaptation: serotonin release within basal ganglia is not rhythmic with movement. In: Massaro, E. J., Schardein, J. L., Broderick, P. A., Schlaepfer, T. E., Mattsson, J. L. (Eds.), Handbook of Neurotoxicology, Vol. 2, Humana Press Inc., Totowa, N.J., pp. 323-367.
Broderick, P. A., 2002. Interleukin 1α alters hippocampal serotonin and norepinephrine release during open-field behavior in Sprague-Dawley animals: differences from the Fawn-Hooded animal model of depression. Prog. Neuro-Psychopharmacol. & Biol. Psychiat. 26, 1355-1372.
Broderick, P. A., Kornak, E. P., Eng, F, Wechsler, R. W., 1993. Real time detection of acute (IP) cocaine-enhanced dopamine and serotonin release in ventrolateral nucleus accumbens of the behaving Norway rat. Pharmacol. Biochem. Behav. 46, 715-722.
Broderick, P. A., Nigro, J. P., Olabisi, O. A., 2001. 5-HT_{2A} antagonist attenuation of cocaine-induced serotonin (5-HT) and dopamine (DA) release in nucleus accumbens (NAcc) during
Broderick, P. A., Pacia, S. V., 2003. Identification, diagnosis and treatment of neuropathologies, neurotoxicities, tumors, and brain and spinal cord injuries using microelectrodes with microvoltammetry. U.S. Patent #PCT/US02/11244.
Broderick, P. A., Pacia, S. V., Doyle, W. K., Devinsky, O., 2000. Monoamine neurotransmitters in resected hippocampal subparcellations from neocortical and mesial temporal lobe epilepsy patients: in situ microvoltammetric studies. Brain Res. 878, 49-63.
Broderick, P. A., Penn Erskine, C., Charleton, H., Green, S., Okonji, C., 1997. A four week follow-up study of serotonin (5-HT) and dopamine (DA) release in nucleus accumbens of animals behaving in an open-field paradigm, after acute cocaine administration. Soc. Neurosci. Abstr. 23, 1867.
Broderick, P. A., Piercey, M. F., 1998a. Clozapine, haloperidol, and the D₄ antagonist, PNU-101387G: in vivo effects on mesocortical, mesolimbic, and nigrostriatal dopamine and serotonin release. J. Neural. Transm, 105, 749-767.
Broderick, P. A., Rahni, D. N., Zhou, Y., 2003. Acute and subacute effects of risperidone and cocaine on accumbens dopamine and serotonin release using in vivo microvoltammetry on line with open-field behavior. Prog. Neuro-Psychopharmacol. & Biol. Psychiat. 27 (6), 1037-1054.
Broderick. P. A., Phelix, C. F., 1997. . Serotonin (5-HT) within dopamine reward circuits signals open-field behavior. II. Basis of 5-HT-DA interaction in cocaine dysfunctional behavior. Neurosci. Biobehav. Rev. 21 (3), 227-260.
Brown, E. E., Finlay, J. M., Wong, J. T., Damsma, G., Fibiger, H. C., 1991. Behavioral and neurochemical interactions between cocaine and buprenorphine: implications for the pharmacology of cocaine abuse. J. Pharmacol. Exp. Therap. 256, 119-126.
Brunello, N., Masotto, C., Steardo, L., Markstein, R., Racagni, G., 1995. New insights into the biology of schizophrenia through the mechanism of action of clozapine. Neuropsychopharmacol. 13 (3), 177-213.
Buckley, P. F., 1998. Substance abuse in schizophrenia: a review. J. Clin. Psychiat. 59 (Suppl. 3), S26-S30.
Callahan, P. M., De La Garza, R., Cunningham, K. A., 1994. Discriminative stimulus properties of cocaine: modification by dopamine D₁ receptors in nucleus accumbens. Psychopharmacology 115, 110-114.
Canton, H., Verriele, L., Colpaert, F. C., 1990. Binding of typical and atypical antipsychotics to 5-HT_{1C} and 5-HT₂ sites: clozapine possibly interacts with 5-HT_{1C} sites. Eur. J. Pharmacol. 191, 93-96.
Carlsson, A., Waters, N., Carlsson, M. L., 1999. Neurotransmitter interactions in schizophrenia-therapeutic implications. Biol. Psychiat. 46 (10), 1388-1395.
Carpenter, W. T., Heinrich, D. W., Wagman, A. M. I., 1988. Deficit and non-deficit forms of schizophrenia, the concept. Am. J. Psychiat. 145, 578-583.
Carroll, F. I., Gray, J. L., Abraham, P., Kuzemko, M. A., Lewin, A. H., Boja, J. W., Kuhar, M. J., 1993. 3-Aryl-2-(3′-substituted-1′,2′,4′-oxadiazol-5′-yl) tropane analogues of cocaine: affinities at the cocaine binding site at the dopamine, serotonin and norepinephrine transporters. J. Med. Chem. 36, 2886-2890.
Carroll, M. E., Lac, S. T., Asencio, M., Kragh, R., 1990a. Intravenous cocaine self-administration in rats is reduced by dietary L-tryptophan. Psychopharmacol. (Berl.) 100, 293-300.
Carroll, M. E., Lac, S. T., Asencio, M., Kragh, R., 1990b. Fluoxetine reduces intravenous cocaine self-administration in rats. Pharmacol. Biochem. Behav. 35, 237-244.
Chen, N. H., Reith, M. E., 1993. 3[H]dopamine and [3H]serotonin release in vitro induced by electrical stimulation in A9 and A10 dopamine regions of rat brain: characterization and responses to cocaine. J. Pharmacol. Exp. Therap. 267 (1), 379-389.
Chou, Y. H., Halidin, C., Farde, L., 2003. Occupancy of 5-HT(1A) receptors by clozapine in the primate brain: a PET study. Psychopharmacol. (Berl.) 166, (3), 234-240.
Church, W. H., Justice, J. B. Jr., Byrd, L. D., 1987. Extracellular dopamine in rat striatum following uptake inhibition by cocaine nomifensine and benztropine. Eur, J. Pharmacol. 139, 325-348.
Clinical Pharmacology of Clozapine. 2002. Rx List of Monographs.
Cohen, S., 1975. Cocaine. JAMA. 231 (1), 74-75.
Conloy, R., Mahmoud, R., 2001. A randomized double-blind study of risperidone and olanzepine in the treatment of schizophrenia or schizoaffective disorder, Am. J. Psychiat. 158, 765-774.
Cools, A. R., van Rossum, J. M., 1970. Caudal dopamine and stereotype behaviour of cats. Arch. Int. Pharmacodyn. 187, 163-173.
Costall, B., Domeney, A. M., Naylor, R. J., 1990. 5-HT₃ receptor antagonists attenuate dopamine-induced hyperactivity in the rat. NeuroReport 1, 77-80.
Costall, B., Naylor, R. J., 1973. The role of telencephalic dopaminergic systems in the mediation of apomorphine-stereotyped behaviour. Eur. J. Pharmacol. 24, 8-24.
Costall, B., Naylor, R. J., Pycock, C., 1976. Non-specific suspersensitivity of striatal dopamine receptors after 6-hydroxydopamine lesions of the nigrostriatal pathway. Eur. J. Pharmacol. 35, 275-283.
Cunningham, K. A., Lakoski, J. M., 1988. Electrophysiological effects of cocaine and procaine on dorsal raphe serotonin neurons. Eur. J. Pharmacol. 148, 457-462.
Dackis, C. A., Gold, M. S., 1985. New concepts in cocaine addiction: the dopamine depletion hypothesis. Neurosci. Biobehav. Rev. 9 (3), 469-477.
Dackis, C. A., Gold, M. S., 1985. New concepts in cocaine addiction: the dopamine depletion hypothesis. Neurosci. Biobehav. Rev. 9, 1-9.
De La Garza, R. 2nd, Cunnigham, K. A., 2000. The effects of the 5-hydroxytryptamine (1A) agonist 8-hydroxy-2-(di-n-propylamino) tetralin on spontaneous activity, cocaine-induced hyperactivity and behavioral sensitization: a microanalysis of locomotor activity. J. Pharmacol. Exp. Therap. 292 (2), 610-617.
de Wit, H., Wise, R. A., 1977. Blockade of cocaine reinforcement in rats with the dopamine receptor blocker pimozide but not with the noradrenergic blockers, phentolamine or phenoxybenzamine. Can. J. Psychol. 31, 195-203.
Delfs, J. M., Schreiber, L., Kelley, A. E., 1990. Microinjection of cocaine into the nucleus accumbens elicits locomotor activation in the rat. J. Neurosci. 10 (1), 303-310.
Devaud, L. L., Hollingsworth, E. B., Cooper, B. R., 1992. Alterations in extracellular and tissue levels of biogenic amines in rat brain induced by the serotonin₂ receptor agonist, ritanserin. J. Neurochem. 59, 1459-1466.
Di Matteo, V., Cacchio, M., Di Giulio, C., Di Giovanni, G., Esposito, E., 2002. Biochemical evidence that the atypical antipsychotic drugs clozapine and risperidone block 5-HT_{2C} receptors in vivo. Pharmacol. Biochem. Behav. 71, 607-613.
Di Matteo, V., Di Giovanni, G., Di Mascio, M., Esposito, E, 1999. SB 242084, a selective serotonin_{2C} receptor antagonist, increases dopaminergic transmission in the mesolimbic system. Neuropharmacol. 38, 1195-1205.
Drescher, K., Hetey, L., 1988. Influence of antipsychotics and serotonin antagonists on presynaptic receptors modulating the release of serotonin in synaptosomes of the nucleus accumbens of rats. Neuropharmacol., 27 (1), 31-36.
Duffy, R. A., Hunt, M. A., Wamsley, J. K., McQuade, R. D., 2000. In vivo autoradiography of [3H]SCH 39166 in rat brain: selective displacement by D1/D5 antagonists. J. Chem. Neuroanat. 19 (1), 41-46
Einhorn, L. C., Johnsen, P. A., White, F. J., 1988. Electrophysiological effects of cocaine in the mesoaccumbens dopamine system: studies in the ventral tegmental area. J. Neurosci. 8, 100-112.
Essman, W. D., Singh, A., Lucki, I., 1994. Serotonergic properties of cocaine: effects on a 5-HT₂ receptor-mediated behavior and on extracellular concentrations of serotonin and dopamine. Pharmacol. Biochem. Behav. 49 (1), 107-113.
Evenden, J. L., Ryan, C. N., 1988. Behavioral responses to psychomotor stimulant drugs: localization in the central nervous system. Pharmac. Ther. 36, 151-172.
Farde, L., Wiesel, F. A., Hallain, C., Sedvall, G., 1988. Central D2-dopamine receptor occupancy in schizophrenia patients treated with antipsychotic drugs. Arch. Gen. Psychiat. 45 (1), 71-76.
Farren, C. K., Hameedi, F. A., Rosen, M. A., Woods, S., Jatlow. P., Kosten, T. R., 2000. Significant interaction between clozapine and cocaine in cocaine addicts. Drug Alcohol Depend. 59 (2), 153-163.
Filip, M., Cunningham, K. A., 2002. Serotonin 5-HT (2c) receptors in nucleus accumbens regulate expression of hyperlocomotive and discriminate stimulus effects of cocaine. Pharmacol. Biochem. Behav. 21 (19) 7781-7787.
Filip, M., Nowak, E., Papia, I., 2001. On the role of serotonin 2A/2C receptors in the sensitization to cocaine. J. Physiol. Pharmacol. 52 (3), 471-481.
Fink-Jensen, A., 2000. Novel pharmacological approaches to the treatment of schizophrenia. Dan. Med. Bull. 47 (3), 151-167.
Fischman, M. W., 1984. The behavioral pharmacology of cocaine in humans. NIDA Res. Mono. 50, 72-91.
Foucault, R., Broderick, P. A., Rahni, D. N., Lombardi, J. R., Birke, R. L., 2002. Neurotransmitter signatures: a correlation between Raman spectroscopy and microvoltammetric BRODERICK PROBES®. NIH/NIGMS Conference & Abstract, New Orleans, La.
Frank, R. A., Tsibulsky, V., Grocki, S., Dashevsky, B., Kehne, J. H., Schmidt, C. J., Sorensen, S. M., 1995. Mixed D₂/5-HT₂ antagonism of amphetamine-induced facilitation of brain stimulation reward. Pharmacol. Biochem. Behav. 52, 799-804.
Galloway, M. P., 1990. Regulation of dopamine and setotonin synthesis by acute administration of cocaine. Synapse 6, 63-72.
Gawin, F., Kleber, H., 1986a. Pharmacological treatments of cocaine abuse. [Review]. Psychiat. Clin. North Am., 9, 573-583.
Gawin, F. H., Allen, D., Humblestone, B., 1989. Outpatient treatment of “crack” cocaine smoking with flupenthixol decanoate. A preliminary report. Arch. Gen. Psychiat. 46, 322-325.
Gawin, F. H., Kleber, H. D., 1986b. Abstinence symptomatology and psychiatric diagnosis in cocaine abusers. Clinical observations. Arch. Gen. Psychiat. 43, 104-113.
Glazer, W. M., 2000. Extrapyramidal side effects, tardive dyskinesia, and the concept of atypicality. J. Clin. Psychiat. 61 (Suppl. 3), S16-S54.
Goeders, N. E., Smith, J. E., 1983. Cortical dopaminergic involvement in cocaine reinforcement. Science 221, 773-774.
Grabowski, J., Rhoades, H., Silverman, P., Schmitz, J. M., Stotts, A., Creson, D., 2000. Risperidone for the treatment of cocaine dependence: randomized, double-blind trial. J. Clin. Psychopharmacol. 20 (3), 305-310.
Hall, F. S., Li, X. F., Sora, I., Xu, F., Caron, M., Lesch, K. P., Murphy, D. L., Uhl, G. R., 2002. Cocaine mechanisms: enhanced cocaine, fluoxetine and nisoxetine place preferences following monoamine transporter deletions. Neuroscience 115 (1), 153-161.
Harris, D., Batki, S. L., 2000. Stimulant psychosis: symptom profile and acute clinical course. Am. J. Addict. 9 (1), 28-37.
Heck, A. H., Haffmans, P. M., de Groot, I. W., Hoencamp, E., 2000. Risperidone versus haloperidol in psychiatric patients with disturbing neuroleptic-induced extrapyramidal symptoms: a double-blind, multi-center trial. Schizophr. Res. 46 (2-3), 97-105.
Hernandez, L., Hoebel, B. G., 1988. Food reward and cocaine increases extracellar dopamine in the nucleus accumbens as measured by microdialysis. Life Sci. 42, 1705-1712.
Herrick-Davis, K., Grinde, E., Niswender, C. M., 1999. Serotonin_{2c} receptor RNA editing alters receptor basal activity: implications for serotonergic signal transduction. J. Neurochem. 73, 1711-1717.
Herrick-Davis, K., Grinde, E., Teitler, M., 2000. Inverse agonist activity of atypical antipsychotic drugs at human 5-hydroxytryptamine_{2c} receptors. J. Pharmacol. Exp. Therap. 295, 226-232.
Hertel, P., Lindblom, N., Nomikos, G. G., Svensson, T. H., 1998. Modulation of central serotonergic neurotransmission by risperidone: underlying mechanism(s) and significance of action. Prog. Neuropsychopharmacol. Biol. Psychiat. 22 (5), 815-834.
Hertel, P., Nomikos, G. G., Iurlo, M., Svensson, T. H., 1996. Risperidone: regional effects in vivo on release and metabolism of dopamine and serotonin in the rat brain. Psychopharmacol. 124, 74-86.
Hertel, P., Nomikos, G. G., Svensson, T. H., 1997b. Risperidone inhibits 5-hydroxy-tryptaminergic neuronal activity in the dorsal raphe nucleus by local release of 5-hydroxy-tryptamine. Br. J. Pharmacol. 122, 1639-1646.
Hertel, P., Nomikos, G. G., Svensson, T. H., 1999. The antipsychotic drug risperidone interacts with auto- and hetero-receptors regulating serotonin output in the rat frontal cortex. Neuropharmacol. 38 (8), 1175-1184.
Herve, D., Pickel, V. M., Joh, T. H., Beaudet, A., 1987. Serotonin axon terminals in the ventral tegmental area of the rat: fine structure and synaptic input to dopaminergic neurons. Brain Res. 435, 71-83.
Herve, D., Simon, H., Blanc, G., Lisoprawski, A., LeMoal, M., Glowinski, J., Tassin, J. P., 1979. Increased utililzation of dopamine in the nucleus accumbens but not in the cerebral cortex after dorsal raphe lesion in the rat. Neurosci. Lett. 15, 127-133.
Hillegaart, V., Wadenberg, M. L., Ahlenius, S., 1989. Effects of 8-OH-DPAT on motor activity in the rat. Pharmacol. Biochem. Behav. 32, 797-800.
Hope, O., Lineen, E., Okonji, C., Green, S., Saleem, A., Aulakh, C. S., Broderick, P. A., 1995. Cocaine has remarkable nucleus accumbens effects on line, with behavior in the serotonin-deficient Fawn Hooded rat. NIH/NIGMS Symposium, Washington, D.C.
Hurd, Y. L., Ungerstedt, U., 1989. Cocaine: an in vivo microdialysis of its acute action on dopamine neurotransmission in rat striatum. Synapse 3, 48-54.
Ichikawa, J., Kuroki, T., Dai, J., Meltzer, H. Y., 1998. Effect of antipsychotic drugs on extracellular serotonin levels in rat medial prefrontal cortex and nucleus accumbens. Eur. J. Pharmacol. 351 (2), 163-171.
Ichikawa, J., Meltzer, H. Y., 2000. The effect of serotonin_{1A} receptor agonism on antipsychotic drug-induced dopamine release in rat striatum and nucleus accumbens. Brain Res. 858 (2), 252-263.
Ishibashi, T., Tagashira, R., Nakamura, M., Noguchi, H., Ohno, Y., 1999. Effects of perospirone, a novel 5-HT2 and D2 receptor antagonist, on FOS protein expression in the rat forebrain. Pharmacol. Biochem. Behav. 63 (4), 535-541.
Izenwasser, S., Wesling, L. L., Cox, B. M., 1990. Comparison of the effects of cocaine and other inhibitors of dopamine uptake in rat striatum nucleus accumbens, olfactory tubercle and medial prefrontal cortex. Brain Res. 520, 303-309.
Janssen, P. A. J., Niemegeers, C. J. E., Awouters, F., Schellekens, K. H. L., Megens, A. A. H. P., Meert, T. F., 1988. Pharmacology of risperidone (R 64766), a new antipsychotic drug with serotonin-S₂ and dopamine-D₂ antagonistic properties. J. Pharmacol. Exp. Therap. 244, 685-693.
Kalivas, P. W., 1993. Neurotransmitter regulation of dopamine neurons in the ventral tegmental area. Brain Res. Rev. 18, 75-113.
Kalivas, P. W., Duffy, P., 1990. Effect of acute and daily cocaine treatment on extracellular dopamine in the nucleus accumbens. Synapse 5, 48-58.
Kalivas, P. W., Nemeroff, C. B., 1988. The mesocorticolimbic dopamine system. New York Acad. Sci., New York.
Kane, J., Honigfeld, G., Singer, J., Meltzer, H., 1988. The Clozaril Collaborative Study Group. Clozapine for the treatment-resistant schizophrenic: a double-blind comparison versus chlorpromazine/benztropine. Arch. Gen. Psychiat. 45, 789-796.
Kane, J. M., 1999. Pharmacologic treatment of schizophrenia. Biol. Psychiat. 46 (10), 1396-1408.
Kapur, S., Remington, G., 2001. Atypical antipsychotics: new directions and new challenges in the treatment of schizophrenia. Ann. Rev. Med. 52, 503-517.
Keegan, D., 1994. Risperidone: neurochemical, pharmacologic and clinical properties of a new antipsychotic drug. Can. J. Psychiat. 39 (Suppl. 2), S46-S52.
Kelly, P. H., Seviour, P. Iversen, S. D., 1975. Amphetamine and apomorphine responses in the rat following 6-OHDA lesions of the nucleus accumbens septi and corpus striatum. Brain Res. 94, 507-522.
King, D. J., 1998. Drug treatment of the negative symptoms of schizophrenia. Eur. Neuropsychopharmacol. 8 (1), 33-42.
Kloss, M. W., Rosen, G. M., Rauckman, E. J., 1984. Biotransformation of norcocaine to norcocaine nitroxide by rat brain microsomes. Psychopharmacol. (Berl.) 84 (2), 221-224.
Koe, B. K., 1976. Molecular geometry of inhibitors of the uptake of catecholamines and serotonin in synaptosomal preparations of rat brain. J. Pharmacol. Exp. Therap. 199, 649-661.
Koe, B. K., 1976. Molecular geometry of inhibitors of the uptake of catecholamines and serotonin in synaptosomal preparations of rat brain. J. Pharmacol. Exp. Therap. 1999, 649-661.
Koob, G. F., Nestler, E. J., 1997. The neurobiology of drug addiction. J. Neuropsychiat. Clin. Neurosci. 9, 482-497.
Kosten, T. A., Nestler, E. J., 1994. Clozapine attenuates cocaine conditioned place preference. Life Sci. 55 (1), PL 9-14.
Kruzich, P. J., See, R. E., 2000. An evaluation of the role of 5-HT(2) receptor antagonism during subchronic antipsychotic drug administration in rats. Brain Res. 875 (1-2), 35-43.
Kuoppamaki, M., Palvimak, E. P., Hietala, J., Syvalahti, E., 1995. Differential regulation of rat 5-HT2A and 5-HT2C receptors, a chronic treatment with clozapine, chlorpromazine and three putative atypical antipsychotic drugs. Neuropsychopharmacol. 13 (2), 139-150.
Kuoppamaki, M., Palvimak, E. P., Hietala, J., Syvalahti, E., 1995. Differential regulation of rat 5-HT2A and 5-HT2C receptors, a chronic treatment with clozapine, chlorpromazine and three putative atypical antipsychotic drugs. Neuropsychopharmacol. 13 (2), 139-150.
Kuroki, T., Meltzer, H. Y., Ichikawa, J., 1999. Effect of antipsychotic drugs on extracellular dopamine levels in rat medial prefrontal cortex and nucleus accumbens. J. Pharmacol. Exp. Therap. 288, 774-781.
Kuroki, T., Meltzer, H. Y., Ichikawa, J., 1999. Effect of antipsychotic drugs on extracellular dopamine levels in rat medial prefrontal cortex and nucleus accumbens. J. Pharmacol. Exp. Therap. 288, 774-781.
Lemmens, P., Brecher, M., Van Baelen, B., 1999. A combined analysis of double-blind studies with risperidone vs. placebo and other antipsychotic agents: factors are associated with extrapyramidal symptoms. Acta. Psychiatr. Scand. 99, 160-170.
Leysen, J. E., Gommeren, W., Een, A., De Chaffoy De Coucelles, J. C., Stoff, J. C., Janssen, P. A. J., 1988. Biochemical profile of risperidone, a new antipsychotic. J. Pharmacol. Exp. Therap. 247, 661-670.
Leysen, J. E., Gommeren, W., Schotte, A., 1996. Serotonin receptor subtypes: possible roles and implications in antipsychotic drug action. In: Kane, J. M., Moller, H.-J., Awouters, R. (Eds.), Serotonergic Mechanisms in Antipsychotic Treatment. Marcel Dekker, New York, pp. 51-76.
Leysen, J. E., Janssen, P. M. F., Gommeren, W., Wynants, J., Pauwels, P. J., Janssen, P. A. J., 1992. In vitro and in vivo receptor binding and effects on monoamine turnover in rat brain regions of the novel antipsychotics, risperidone and ocaperidone. Mol. Pharmacol. 41, 494-508.
Leysen, J. E., Janssen, P. M. F., Schotte, A., Luyten, W. H. M. L., Megens, A. A. H. P., 1993. Interaction of antipsychotic drugs with neurotransmitter receptor sites in vitro and in vivo in relation to pharmacological and clinical effects: role of 5-HT₂ receptors. Psychopharmacol. 112 (Suppl.), S40-S54.
Lieberman, J., Johns, C., Cooper, T., Pollack, S., Kane, J., 1989. Clozapine pharmacology and tardive dyskinesia. Psychopharmacol. (Berl.) 99 (Suppl.), S54-S59.
Lieberman, J. A., Kinon, B. J., Loebel, A. D., 1990. Dopaminergic mechanisms in idiopathic and drug-induced psychoses. Schizophr. Bull. 16 (1), 97-110.
Lieberman, J. A., Mailman, R. B., Duncan, G., Sikich, L., Chakos, M., Nichols, D. E., Kraus, J. E., 1998. Serotonergic basis of antipsychotic drug effects in schizophrenia. Biol. Psychiat. 44 (11), 1099-1117.
Loh, E. A., Fitch, T., Vickers, G., Roberts, D. C., 1992. Clozapine increases breaking points on a progressive-ratio schedule reinforced by intravenous cocaine. Pharmacol. Biochem. Behav. 42 (3), 559-562.
Loh, E. A., Roberts, D. C., 1990. Breakpoints on a progressive-ratio schedule reinforced by intravenous cocaine increase following depletion of forebrain serotonin. Psychopharmacol. 101, 262-266.
Lucas, G., Spampinato, U., 2000. Role of striatal serotonin_{2A} and serotonin_{2C} receptor subtypes in the control of in vivo dopamine outflow in the rat striatum. J. Neurochem. 74, 693-701.
Lutje Hulsik, D., 2002. Functional and binding data for ketanserin. Organon.
Lysaker, P., Bell, M., Beam-Goulet, J., Milstein, R., 1994. Relationship of positive and negative symptoms to cocaine abuse in schizophrenia. J. Nerv. Ment. Dis. 182 (2), 109-112.
Mancama, D., Arranz, M. J., Munro, J., Osborne, S., Makoff, A., Collier, D., Kerwin, R., 2002. Investigation of promoter variants of the histamine 1 and 2 receptors in schizophrenia and clozapine response. Neurosci. Lett. 333 (3), 207-211.
Mannens, G., Huang, M. L., Meuldermans, W., Hendrickx, J., Woestenborghs, R., Heykants, J., 1993. Absorption, metabolism and excretion of risperidone in humans. Drug Metab. Dispos. 21, 1134-1141.
Marder, S. R., Meibach, R. C., 1994. Risperidone in the treatment of schizophrenia. Am. J. Psychiat. 15, 825-835.
Margolin, A., Avants, S. K., Kosten, T. R., 1995. Mazindol for relapse prevention to cocaine abuse in methadone-maintained patients. Am. J. Drug Alcohol Abuse 21 (4), 469-481.
McGregor, A., Lacosta, S., Roberts, D. C. S., 1993. L-Tryptophan decreases the breaking point under a progressive ratio schedule of intravenous cocaine self-administration in the rat. Pharmacol. Biochem. Behav. 45, 651-655.
McKinney, W. T., 1989. Basis of development of animal models in psychiatry: an overview. In: Koob, G. F., Elhers, C. L., Kupfer, D. J. (Eds.), Animal Models of Depression. Birkhauser Press, Boston, pp. 3-17.
McKinzie, D. L., Rodd, Z. A., Dagon, C. T., Murphy, J. M., McBride, W. J., 1999. Cocaine is self-administered into the shell region of the nucleus accumbens in Wistar rats. In: McGinty, J. F. (Ed), Advancing from the ventral striatum to the extended amygdala: implications for neuropsychiatry and drug abuse. NY Acad. Sci., New York, N.Y.
McMahon, L. R., Cunningham, K. A., 1999. Antagonism of 5-hydroxytryptamine (4) receptors attenuates hyperactivity induced by cocaine: putative role for 5-hydroxy-tryptamine (4) receptors in the nucleus accumbens shell. J. Pharmacol. Exp. Therap. 291 (1), 300-307.
McMahon, L. R., Cunningham, K. A., 2001. Antagonism of 5-hydroxytryptamine (2A) receptors attenuates the behavioral effects of cocaine in rats. J. Pharmacol. Exp. Therap. 297 (1), 357-363.
McMahon, L. R., Filip, M., Cunningham, K. A., 2001. Differential regulation of the mesoaccumbens circuit by serotonin 5-hydroxytryptamine (5-HT)2A and (5-HT)2C receptors. J. Neurosci. 21 (19), 7781-7787.
Megens, A. A., Awouters, F. H., Meert, T. F., Schellekens, K. H., Niemegeers, C. J., Janssen, P. A., 1992. Pharmacological profile of the new potent neuroleptic, ocaperidone (R 79598). J. Pharmacol. Exp. Therap. 260 (1), 146-159.
Meltzer, H. Y., 1989. Clinical studies on the mechanism of action of clozapine: the dopamine-serotonin hypothesis of schizophrenia. Psychopharmacol. 99, 518-527.
Meltzer, H. Y., 1991. The mechanism of action of novel antipschotic drugs. Schizophr. Bull. 17, 263-287.
Meltzer, H. Y., 1992. Dimensions of outcome with clozapine. Br. J. Psychiat. 160 (Suppl. 17), S46-S53.
Meltzer, H. Y., 1999. The role of serotonin in antipsychotic drug action. Neuropsychopharmacol. 21 (Suppl.), S106-S115.
Meltzer, H. Y., Nash, J. F., 1991. Effects of antipsychotic drugs on serotonin receptors. Pharmacol. Rev. 43, 587-604.
Meltzer, H. Y., Zhang, Y., Stockmeior, C. A., 1992. Effect of amperozide on rat cortical 5-HT₂ and striatal and limbic dopamine D₂ receptor occupancy: implications for antipsychotic action. Eur. J. Pharmacol. 216, 67-71.
Mendoza, R., Miller, B. L. and Mena, I., 1992. Emergency room evaluation of cocaine-associated neuropsychiatric disorders. Recent Dev. Alcohol 10, 73-87.
Mets, B., Diaz, J., Soo, E., Jamdar, S., 1999. Cocaine, norcocaine, ecgonine methylester and benzoylecgonine pharmacokinetics in the rat. Life Sci. 65 (12), 1317-1328.
Miller, B. L., Mena, I., Giombetti, R., Villanueve-Meyer, J. Djenderedjian, A. H., 1992. Neuropsychiatric effects of cocaine: SPECT Measurements. J. Addict. Dis. 11, 47-58.
Misra, A. L., Nayak, P. K, Patel, M. N., Vadlamani, N. I., Mule, S. J., 1974a. Identification of norcocaine as a metabolite of (3H)-cocaine in rat brain. Experientia 30 (11), 1312-1314.
Misra, A. L., Vadlamani, N. I., Bloch, R., Nayak, P. K., Mule, S. J., 1974b. Physiologic disposition and metabolism of (3H) ecgonine (cocaine metabolite) in the rat. Res. Commun. Chem. Pathol. Pharmacol. 8 (1), 55-63.
Mitchell, J., Vierkant, A. D., 1991. Delusions and hallucinations of cocaine abusers and paranoid schizophrenics: a comparative study. J. Psychol. 125 (3), 301-310.
Moser, P. C., Morgan, P. M., Frank, R. A., Kehne, J. H., 1995. Reversal of amphetamine-induced behaviors by MDL 100,907, a selective 5-HT_{2A} antagonist. Behav. Br. Res. 73, 163-167.
Mueser, K. T., Nishith, P., Tracy, H., De Girolamo, J., Molinaro, M., 1995. Expectations and motives for substance use in schizophrenia. Schizophr. Bull. 21 (3), 367-378.
Mukherjee, J., Christian, B. T., Narayanan, T. K., Shi, B., Mantil, J., 2001. Evaluation of dopamine d-2 receptor occupancy by clozapine, risperidone and haloperidol in vivo in the rodent and non-human primate brain using 18f-fallypride. Neuropsychopharmacol. 25 (4), 476-488.
Myers, J. E., Thase, M. E., 2001. Risperidone: review of its therapeutic utility in depression. Psychopharm. Bull. 35 (4), 109-129.
Nambudin, D. E. and Young, R. C., 1991. A case of late-onset crack dependence and subsequent psychosis in the elderly. J. Subst. Abuse Treat. 8, 253-255.
Nayak, P. K., Misra, A. L., Mule, S. J., 1976. Physiological disposition and biotransformation of (3H) cocaine in acutely and chronically treated rats. J. Pharmacol. Exp. Therap. 196 (3), 556-569.
Newton, T. F., Ling, W., Kalechstein, A. D., Uslaner, J., Tervo, K., 2001. Risperidone pre-treatment reduces the euphoric effects of experimentally administered cocaine. Psychiat. Res. 102 (3), 227-233. Ng, J. P, Hubert, G. W., Justice, J. B. Jr., 1991. Increased stimulated release and uptake of dopamine in nucleus accumbens after repeated cocaine administration as measured by in vivo voltammetry. J. Neurochem. 56, 1485-1492.
open-field behavior: in vivo microvoltammetric studies using ketanserin. Soc. Neurosci. Abstr. 27, 405.
Pan, Z. Z., William, J. T., 1989. Differential actions of cocaine and amphetamine on dorsal raphe neurons in vitro. J. Pharmacol. Exp. Therap. 251, 56-62.
Parsa, M., Ramirez, L. F., Loula, E. C., Meltzer, H. Y., 1991. Effect of clozapine on psychotic depression and parkinsonism. J. Clin. Psychopharmacol. 11, 330-331.
Parsons, L. H., Justice, J. B., 1993. Serotonin and dopamine sensitization in the nucleus accumbens, ventral tegmental area and dorsal raphe nucleus following repeated cocaine administration. J. Neurochem. 61, 1611-1619.
Parsons, L. H., Koob, G. F., Weiss, F. 1995. Serotonin dysfunction in the nucleus accumbens of rats during withdrawal after unlimited access to intravenous cocaine. J. Pharmacol. Exp. Therap. 274, 1182-1191.
Parsons, L. H., Koob, G. F., Weiss, F., 1996. Extracellular serotonin is decreased in the nucleus accumbens during withdrawal from cocaine self-administration. Behav. Br. Res. 73 (1-2), 225-228.
Pellegrino, L. J., Pellegrino, A. S., Cushman, A. J., 1979. A Stereotaxic Atlas of the Rat Brain. Plenum Press, New York.
Peltier, R. L., Emmett-Oglesby, M. W., Thomas, W. H., Schenk, S., 1994. Failure of ritanserin to block the discriminative or reinforcing stimulus effects of cocaine. Pharmacol. Biochem. Behav. 48, 473-478.
Pere, J. J., 1995. Clinical pharmacology: the example of clozapine (Leponex). Encephale 21 (3), 9-12.
Phelix, C. F., Broderick, P. A., 1995. Light microscopic immunocytochemical evidence of converging serotonin and dopamine terminals in ventrolateral nucleus accumbens. Brain Res. Rev. 37, 37-40.
Pijnenburg, A. J. J., Honig, W. M. M., van Rossum, J. M., 1975. Effects of antagonists upon locomotor stimulation induced by injection of dopamine and noradrenaline into the nucleus accumbens of nialamide-pretreated rats. Psychopharmacologia. 41, 175-180.
Porrino, L. J., Ritz, M. C., Goodman, N. L., Sharpe, L. G., Kuhar, M. J., Goldberg, S. R., 1989. Differential effects of the pharmacological manipulation of serotonin systems on cocaine and amphetamine self-administration in rats. Life Sci. 45, 1529-1535.
Price, L. H., Charney, D. S., Delgado, P. L., Heninger, G. R., 2001. Lithium and serotonin function: implications for the serotonin hypothesis of depression. Psychopharmacol. (Berl.) 100, 3-12.
Ramana, S. V., Desiraju, T., 1989. Investigation of influence of diazepam, valproate, cyproheptadine and cortisol on the rewarding ventral tegmental self-stimulation behavior. Ind. J. Physiol. Pharmacol. 33, 179-185.
Ranjan, R., Meltzer, H. Y., 1996. Acute and long-term effectiveness of clozapine in treatment-resistant psychotic depression. Biol. Psychiat. 40, 253-258.
Reith, M. E., Li, M. Y., Yan, Q. S., 1997. Extracellular dopamine, norepinephrine and serotonin in the ventral tegmental area and nucleus accumbens of freely moving rats during intracerebral dialysis following systemic administration of cocaine and other uptake blockers. Psychopharmacology (Berl.) 134, 309-317.
Reith, M. E., Sershen, H., Allen, D. L., Lajtha, A., 1983. A portion of [³H] cocaine binding in brain is associated with serotonin neurons. Mol. Pharmacol. 23, 600-606.
Richardson, N. R., Roberts, D. C. S., 1991. Fluoxetine pretreatment reduces breaking point on a progressive ratio schedule reinforced by intravenous cocaine self-administration in the rat. Life Sci. 49, 833-840.
Richelson E., Souder, T., 2000. Binding of antipsychotic drugs to human brain receptors focus on newer generation compounds. Life Sci. 68 (1), 29-39.
Risner, M. E., Jones, B. E., 1980. Intravenous self-administration of cocaine and norcocaine by dogs. Psychopharmacol. 71, 83-89.
Ritz, M. C., Lamb, R. J., Goldberg, S. R., Kuhar, M. J., 1987. Cocaine receptors on dopamine transporters are related to self-administration of cocaine. Science 237, 1219-1223.
Roberts, D. C. S., Corcoran, M. E. O., Fibiger, H. C., 1977. On the role of ascending catecholaminergic systems in intravenous self-administration of cocaine. Pharmacol. Biochem. Behav. 6, 615-620.
Roberts, D. C. S., Koob, G. F., 1982. Disruption of cocaine self-administration following 6-hydroxydopamine lesions of the ventral tegmental area in rats. Pharmacol. Biochem. Behav. 17, 901-904.
Roberts, D. C. S., Koob, G. F., 1982. Disruption of cocaine self-administration following 6-hydroxydopamine lesions of the ventral tegmental area in rats. Pharmacol. Biochem. Behav. 17, 901-904.
Robertson, A., MacDonald, C., 1986. The effects of some atypical neuroleptics on apomorphine-induced behaviors as a measure of their relative potencies in blocking presynaptic versus postsynaptic dopamine receptors. Pharmacol. Biochem. Behav. 24 (6), 1639-1643.
Rosenthal, R. N., Miner, C. R., 1997. Differential diagnosis of substance-induced psychosis and schizophrenia in patients with substance use disorders. Schizophr. Bull. 23 (2), 187-193.
Ross, S. B., Renyi, A. L., 1969. Inhibition of the uptake of tritiated 5-hydroxytryptamine in brain tissue. Eur. J. Pharmacol. 7, 270-277.
Rosse, R. B., Collins, J. P., Jr., Fay-McCarthy, M., Alim, T. M., Wyatt, R. J., Deutsch, S. I., 1994. Phenomenologic comparison of the idiopathic psychosis of schizophrenia and drug-induced cocaine and phencyclidine psychoses: a retrospective study. Clin. Neuropharmacol. 17, 359-369.
Ryan, L., Martone, M., Linder, J., Groves, P., 1988. Cocaine, in contrast to d-amphetamine, does not cause axonal terminal degeneration in neostriatum and agranular frontal cortex of Long Evans rats. Life Sci. 43, 1403-1409.
Satel, S. L., Edell, W. S., 1991. Cocaine-induced paranoia and psychosis proneness. Am. J. Psychiat. 148 (12), 1708-1711.
Satel, S. L., Seibyl, J. P., Charney, D. S., 1991. Prolonged cocaine psychosis implies underlying major psychopathology. J. Clin. Psychiat. 52 (8), 349-350.
Satel, S. L., Seibyl, J. P., Charney, D. S., 1991. Prolonged cocaine psychosis implies underlying major psychopathology. J. Clin. Psychiat. 52, 349-350.
Schotte, A., Janssen, P. F., Megens, A. A., Leysen, J. E., 1993. Occupancy of central nerurotransmitter receptors by risperidone, clozapine and haloperidol, measured ex vivo by quantitative autoradiography. Brain Res. 631 (2), 191-202.
Schoftte, A., Janssen, P. F. M., Gommeren, W., Luyten, W. H. M. L., Van Gompel, P., Lesage, A. S., De Loore, K., Leysen, J. E., 1996. Risperidone compared with new and reference antipsychotic drugs: in vitro and in vivo receptor binding. Psychopharmacol. 124, 57-73.
Schotte, A., Janssen, P. F. M., Gommeren, W., Luyten, W. H. M. L., Van Gompel, P., Lesage, A. S., De Loore, K., Leysen, J. E., 1996. Risperidone compared with new and reference antipsychotic drugs: in vitro and in vivo receptor binding. Psychopharmacol. 124, 57-73.
Schotte, A., Janssen, P. F. M., Megens, A. A. U. P., Leysen, J. E., 1993. Occupancy of central nerurotransmitter receptors by risperidone, clozapine and haloperidol, measured ex vivo by quantitative autoradiography. Brain Res. 631, 191-202.
Seecof, R., Tennant, F. S. Jr., 1986. Subjective perceptions to the intravenous “rush” of heroin and cocaine in opioid addicts. Am. J. Drug Alcohol Abuse. 12 (1-2), 79-87.
Seeman, P., 1987. Dopamine receptors and the dopamine hypothesis of schizophrenia. Synapse 1, 133.
Seiden, L. S., Kleven, M. S., 1988. Lack of toxic effects on dopamine and serotonin neurons in rat brain, In: Clouet, D., Asghar, K., Brown, R. (Eds.), Mechanisms of Cocaine Abuse and Toxicity. DHHS Pub #(ADM) 88-1585, Rockville, Md., pp. 276-289.
Serper, M. R., Chou, J. C., Allen, M. H., Czobor, P., Cancro, R., 1999. Symptomatic overlap of cocaine intoxication and acute schizophrenia at emergency presentation. Schizophr. Bull. 25 (2), 387-394.
Sherer, M. A., Kumor, K. M., Cone, E. J., Jaffe, J. H., 1988. Suspiciousness induced by four-hour intravenous infusions of cocaine. Preliminary findings. Arch. Gen. Psychiat. 45 (7), 673-677.
Smelson, D. A., Losonczy, M. F., Davis, C. W., Kaune, M., Williams, J., Ziedonis, D., 2002. Risperidone decreases craving and relapses in individuals with schizophrenia and cocaine dependence. Can. J. Psychiat. 47 (7), 671-675.
Smelson, D. A., Roy, A., Roy, M., 1997. Risperidone diminishes cue-elicited craving in withdrawn cocaine-dependent patients. Can. J. Psychiat. 42 (9), 984.
Sokoloff, P., Giros, B., Martres, M. P., Boutheret, M. L., Schwartz, J. C., 1990. Molecular cloning and characterization of a novel dopamine receptor (D₃) as a target for neuroleptics. Nature 347, 146-151.
Spealman, R. D., Bergman, J., Madras, B. K., Kamien, J. B., Melia, K. F., 1992. Role of D1 and D2 dopamine receptors in the behavioral effects of cocaine. Neurochem. Int. 20, (Suppl.), S147-S152.
Sprouse, J. S., Reynolds, L. S., Braselton, J. P., Rollema, H., Zorn, S. H., 1999. Comparison of the novel antipsychotic ziprasidone with clozapine and olanzepine: inhibition of dorsal raphe cell firing and the role of 5-HT(1A) receptor activation. Neuropsychopharmacol. 21 (5), 622-631.
Stahl, S. M., 2000. Essential Psychopharmacology, Second ed. Cambridge University Press, New York.
Steinbusch, H. W. M., 1981. Distribution of serotonin-immunoreactivity in the central nervous system of the rat-cell bodies and terminals. Neurosci. 6, 557-618.
Sumiyoshi, T., Kide, H., Sakamoto, H., Uresaki, K., Suzuki, K., Yamaguchi, N., Mori, H., Shiba, K., Yokogawa, K., 1994. In vivo dopamine-D₂ and serotonin-5-HT₂ receptor binding study of risperidone and haloperidol. Pharmacol. Biochem. Behav. 47, 553-557.
Sumiyoshi, T., Suzuki, K., Sakamoto, H., Yamaguchi, N., Hirofumi, M., Shiba, K., Yokogawa, K., 1995. Atypicality of several antipsychotics on the basis of dopamine-D₂ and serotonin-5-HT₂ receptor occupancy. Neuropsychopharmacol. 12, 57-64.
Sun, L., Lau, C. E., 2001. Simultaneous pharmacokinetic modeling of cocaine and its metabolites, norcocaine and benzoylecgonine, after intravenous and oral administration in rats. Drug Metab. Dispos. 29 (9), 1183-1189.
Svartergren, J., Celandar, M., 1994. The limbic functional selectivity of amperozide is not mediated by dopamine D₂ receptors as assessed by in vitro and in vivo binding. Eur. J. Pharmacol. 254, 73-81.
Tarazi, F. I., Zhang, K., Baldessarini, R. J., 2000. Olanzepine, quetiapine, and risperidone: long-term effects on monoamine transporters in rat forebrain. Neurosci. Lett. 287, 81-84.
Taylor, W. A., Staby, A. E., 1992. Acute treatment of alcohol and cocaine emergencies. Recent Dev. Alcohol. 10, 179-191.
Teneud, L. M., Baptista, T., Murzi, E., Hoebel, B. G., Hernandez, L., 1996. Systemic and local cocaine increase extracellular serotonin in the nucleus accumbens. Pharmacol. Biochem. Behav. 53 (3), 747-752.
Tsibulsky, V. L., Grocki, S., Dashevsky, B. A., Kehne, J. H., Schmidt, C. J., Sorenson, S. M., Frank, R. A., 1998. Mixed D₂/5-HT_{2A} antagonism of cocaine-induced facilitation of brain stimulation reward. Pharmacol. Biochem. Behav. 59 (2), 275-280.
Tsuang, J. W., Eckman, T., Marder, S., Tucker, D., 2002. Can risperidone reduce cocaine use in substance abusing schizophrenic patients? J. Clin. Psychopharmacol. 22 (6), 629-630.
Tueth, M. J., 1993. High incidence of psychosis in cocaine intoxication and preventing violence in the ED. Am. J. Emerg. Med. 11 (6), 676.
Turgay, A., Binder, C., Snyder, R., Fisman, S., 2002. Long term safety and efficacy of risperidone for the treatment of disruptive behavior disorders in children with sub-average IQ's. Pediatrics 110 (3) e 34.
Van Bockstaele, E. J., Cestari, D. M., Pickel, V. M., 1994. Synaptic structure and connectivity of serotonin in the ventral tegmental area: potential sites for modulation of mesolimbic dopamine neurons. Brain Res. 674, 307-322.
Van Bockstaele, E. J., Pickel, V. M., 1993. Ultrastructure of serotonin-immunoreactive terminals in the core and shell of the rat nucleus accumbens: cellular substrates for interactions with catecholamine afferents. J. Comp. Neurol. 334, 603-617.
Van Campenhout, N., De Haes, P., Meer, T. F., 1999. Inability of antipsychotics to antagonize the cueing properties of cocaine in rats. Pharmacol. Biochem. Behav. 64 (2), 435-438.
Volonte, M., Monferini, E., Cerutti, M., Fodritto, F., Borsini, F., 1997. BIMG 80, a novel potential antipsychotic drug: evidence for multireceptor actions and preferential release of dopamine in prefrontal cortex. J. Neurochem, 69 (1), 182-190.
Wadenberg, M. L., Sollman, A., VanderSpek, S. C., Kapur, S., 2001. Dopamine d (2) receptor occupancy is a common mechanism underlying animal models of antipsychotics and their clinical effects. Neuropsychopharmacol. 25 (5), 633-641.
Walsh, S. L., Cunningham, K. A., 1997. Serotonergic mechanisms involved in the discriminative stimulus, reinforcing, and subjective effects of cocaine. Psychopharmacol. 130, 41-58.
Waters, N., Lagerskvist, S., Lofberg, L., Piercey, M. F., Carlsson, A., 1993. The dopamine D3 receptor and autoreceptor preferring antagonists (+)-AJ76 and (+)-UH232: a microdialysis study. Eur. J. Pharmacol. 242, 151-163.
Weinberger, D. R., Berman, F. B., Suddath, R., Fuller-Torrey, E., 1992. Evidence of dysfunction of a prefrontal-limbic network in schizophrenia: a magnetic imaging and regional cerebral blood flow study of discordant monozygotic twin. Am. J. Psychiat. 149, 890-897.
Weiner, I., Gaisler, I., Schiller, D., Green, A., Zuckerman, L., Joel, D., 2000. Screening of antipsychotic drugs in animal models. Drug Dev. Res. 50, 235-249.
Williams, R., 2001. Optimal dosing with risperidone: updated recommendations. J. Clin. Psychiat. 62 (4), 282-289.
Wise, R. A., 1995. D₁- and D₂-type contributions to psychomotor sensitization and reward: implications for pharmacological treatment strategies. Clin. Neuropharmacol. 18 (Suppl. 1), S74-S83.
Wise, R. A., Bozarth, M. A., 1987. A psychostimulant theory of addiction. Psychol. Rev. 94, 469-492.
Wise, R. A., Rompre, P. P., 1989. Brain dopamine and reward. Ann. Rev. Psychol. 40, 191-225.
Wright, I. K., Gariat, J. C., Marsden, C. A., 1990. Effects of a selective 5-HT₂ agonist, DOI, on 5-HT neuronal firing in the dorsal raphe nucleus and 5-HT release and metabolism in the frontal cortex. Br. J. Pharmacol. 99, 221-223.
Yan, Q. S., 2000. Activation of 5-HT_{2A/2C} receptors within the nucleus accumbens increases local dopaminergic transmission. Brain Res. Bull. 51 (1), 75-81.
Yan, Q. S., Maarten, E. A., Yan, S., 2000. Enhanced accumbal dopamine release following 5-HT_{2A} receptor stimulation in rats pretreated with intermittent cocaine. Brain Res. 863 (1-2), 254-258.
Yeats, W. B., 1956. The collected poems of W. B. Yeats. Macmillan, New York, N.Y.
Zahniser, N. R., Peris, J., Dwoskin, L. P., Curella, P., Yasuda, R. P., O'Keefe, L., Boyson, S. J., 1988. Sensitization to cocaine in the nigrostriatal dopamine system. NIDA Res. Monogr. 88, 55-77.
Zimmet, S. V., Strous, R. D., Burgess, E. S., Kohnstamm, S., Green, A. L., 2000. Effects of clozapine on substance use in patients with schizophrenia and schizoaffective disorder: a retrospective survey. J. Clin. Psychopharmacol. 20 (1), 94-98.

I claim:

1. A method of treating cocaine-induced psychosis in a mammal comprising administering an atypical antipsycotic compound to said mammal in an amount sufficient to increase serotonin concentration in the nucleus accumbens of said mammal's brain.

2. The method of claim 1, wherein the antipsychotic compound is selected from the group consisting of clozapine, risperidone, olanzepine, quetiapine, ziprasidone, sertindole, ketanserin, aripiprazole, and haloperidol, flupenthixol, thioridazine, loxapine, fluspirilene, and sulpiride.

3. The method of claim 1, wherein the antipsychotic compound is clozapine.

4. A method of increasing the concentration of serotonin in the nucleus accumbens of a mammalian brain during or following cocaine-induced psychosis comprising administering a serotonin-increasing amount of an atypical antipsychotic compound.

5. The method of claim 4, wherein the antipsychotic compound is selected from the group consisting of clozapine, risperidone, olanzepine, quetiapine, ziprasidone, sertindole, ketanserin, aripiprazole, and haloperidol, flupenthixol, thioridazine, loxapine, fluspirilene, and sulpiride.

6. The method of claim 4, wherein the antipsychotic compound is clozapine.
